(19) United States
(12) Patent Application Publication
(10) Pub. No.: US 2008/0153799 A1
(43) Pub. Date: Jun. 26, 2008

(54) Novel Quinoline-Carbaxamides as Jak3 Kinase Modulators

(75) Inventors: David Laurent, Lund (SE); Jorgen Gustafsson, Lund (SE); Karolina Lawitz, Lund (SE); Tesfaledet Mussie, Lund (SE); Antonios Nikitidis, Lund (SE)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 10/597,896

(22) PCT Filed: Feb. 7, 2005
(86) PCT No.: PCT/SE2005/000156

§ 371 Date: Aug. 10, 2006

(30) Foreign Application Priority Data
Feb. 10, 2004 (SE) 0400284-6

(51) Int. Cl. A61K 31/397; C07D 215/00; A61K 31/47; C07D 295/00; C07D 217/00; A61P 37/00; A61K 31/5377; A61K 31/541; A61K 31/496

(52) U.S. Cl. 514/210.18; 546/159; 514/313; 544/128; 514/235.2; 544/62; 514/228.2; 544/363; 514/253.06; 544/121; 546/139; 514/307

(57) ABSTRACT

The present invention relates to novel compounds which are JAK3 Kinase inhibitors, methods for their preparation and pharmaceutical compositions comprising them. A compound of formula (I) or a pharmaceutically acceptable salt or solvate thereof, wherein X is —CHOH or —C═O;

The present invention relates to novel compounds which are JAK3 Kinase inhibitors, processes for their preparation, pharmaceutical compositions containing them and their use in therapy.

Janus Kinase 3 (JAK3) is a member of the Janus family of protein kinases. Although the other members of this family are expressed by essentially all tissues, JAK3 expression is limited to hematopoetic cells. This is consistent with its essential role in signaling through the receptors for IL-2, IL-4, IL-7, IL-9, IL-13 and IL-15 by non-covalent association of JAK3 with the gamma chain common to these multichain receptors. These cytokines all have a shared function in that they are involved in lymphocyte differentiation and proliferation. XSCID patient populations have been identified with severely reduced levels of JAK3 protein or with genetic defects to the common gamma chain, suggesting that immunosupression should result from blocking signaling through the JAK3 pathway. Animal studies have suggested that JAK3 not only play a critical role in B- and T-lymphocyte maturation, but that JAK3 is constitutively required to maintain T-cell function. Modulation of immune activity through this novel mechanism can prove useful in the treatment of T-cell proliferative disorders such as transplant rejection and autoimmune diseases.

The role of JAK3 in mast cells has been described in knockout mice. Thus, IgE/antigen induced degranulation and mediator release were substantially reduced in mast cells generated from JAK3 deficient mice. JAK3 deficiency does not affect mast cell proliferation in vitro, it has also been shown that IgE receptor levels and mediator contents are identical in JAK3−/− and JAK3+/+ mast cells. Therefore, JAK3 appears essential for the complete response of IgE challenged mast cells. The role of JAK3 in mast cell activation has been well established in murine system, however, there is no published data on mast cell function in the AR-SCID patients. Targeting JAK3 provides the basis for new and effective treatment of mast cell mediated allergic reactions.

JAK3 inhibitors which have been disclosed to date include quinazolines (Sudbeck, E. A. et al. Clinical Cancer Res. 5 (1999) 1569-82, WO 00/0202) and pyrrolo[2,3-d]pyrimidines (Blumenkopf, T. A. et al. WO 99/65909). 4-anilinoquinoline-3-carboxamides having JAK3 inhibitory activity are described in WO 02/092571. In WO 00/18761 and WO 98/43960 there are disclosed substituted quinoline-3-carbonitrile derivatives which are stated to have kinase inhibitory activity.

Quinoline derivatives having other pharmaceutical uses, for example as antiulcer agents, phosphodiesterase inhibitors or gastric acid secretion inhibitors are described in EP 0 259 174, EP 0 346 208, EP 0 480 052 and WO 2004/103998.

The present invention provides a compound of formula (I)

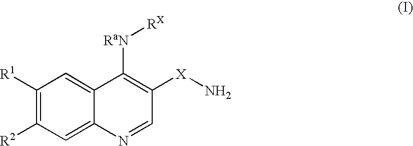

or a pharmaceutically acceptable salt or solvate thereof, wherein

### X is —CHOH or —C═O;

R¹ and R², which may be the same or different, represent nitro, cyano, C₁-C₈ alkyl, C₁-C₈ alkoxy, hydroxy, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pR⁶, Y(CR³₂)ₚOCOR⁶
or R¹ and R² are linked together as —OCH₂O— or —OCH₂CH₂O—;
R³ groups are independently hydrogen, C₁-C₈ alkyl, hydroxy, C₁-C₈ alkoxy or halogen;
p is 0, 1, 2, 3, 4 or 5;
Y is oxygen, CH_{2′}—OSO₂— or NR⁷
R⁴ and R⁵ each independently represent hydrogen or a group selected from C₁-C₈ alkyl, —C₁-C₈ alkoxy, —CO—(C₁-C₈)alkyl, —CO—(C₁-C₈) cycloalkyl, —SO₂—(C₁-C₈)alkyl, —CO—(C₁-C₈) alkoxy, —CO—NR⁷(C₁-C₈)alkyl, C₃-C₈ cycloalkyl, each of which groups may optionally be substituted by one or more hydroxy, cyano, —CONH₂ or —CO—(C₁-C₈) alkoxy groups, or R⁴ and R⁵ together with the nitrogen atom to which they are attached form a 4- to 7-membered, saturated or aromatic heterocyclic ring system optionally containing one or more additional heteroatoms selected from oxygen, sulphur or nitrogen, the ring itself being optionally substituted by at least one substituent selected from hydroxy, C₁-C₈ alkyl, ═O, C₁-C₈ alkoxy or (C₁-C₈ alkoxy)-CO—, or one of R⁴ and R⁵ is hydrogen or C₁-C₈ alkyl and the other is a 5- or 6-membered heterocyclic ring system optionally containing a further oxygen, sulphur or nitrogen atom;
R⁶ is hydrogen, C₁-C₈ alkyl (itself optionally substituted by one or more hydroxy, cyano, halogen or amino groups), phenyl, benzyl, —CO(C₁-C₈)alkyl or a saturated monocyclic 4- to 7-membered ring, which ring may optionally comprise one or more heteroatoms selected from nitrogen, oxygen and sulphur, the ring itself being optionally substituted by at least one substituent selected from C₁-C₈ alkyl, C₁-C₈ alkoxy, ═O, C₁-C₈ alkyl —CO—, or (C₁-C₈ alkoxy)-CO— where any C₁-C₈ alkyl is optionally substituted by one or more hydroxy, cyano, halogen or amino groups;
R⁷ is hydrogen or C₁-C₈ alkyl;

R^{a} is hydrogen or C₁-C₈ alkyl;

R^{x} is a group selected from C₁-C₈ alkyl, C₃-C₈ cycloalkyl or a saturated monocyclic 4- to 7-membered ring comprising one or more heteroatoms selected from nitrogen, oxygen and sulphur, wherein any C₃-C₈ cycloalkyl group or saturated monocyclic 4- to 7-membered ring is optionally substituted by one or more groups selected from hydroxy, azido, cyano, amino, halogen, —CONH₂—, C₁-C₈ alkyl, (C₁-C₈ alkyl)CO—, C₁-C₈ alkoxy, or (C₁-C₈ alkoxy)-CO—, and any C₁-C₈ alkyl, C₁-C₈ alkyl)CO—, C₁-C₈ alkoxy, or (C₁-C₈ alkoxy)-CO— group is itself optionally substituted by one or more substituents selected from hydroxy, azido, cyano, amino, halogen or phenyl; or R^{x} is a group Ar;
Ar is selected from phenyl, tetrahydronaphthenyl, indolyl, pyrazolyl, dihydroindenyl, 1-oxo-2,3-dihydroindenyl, indazolyl, dihydroisoquinolyl, oxodihydroisoquinolyl, tetrahydroisoquinolyl or oxotetrahydroisoquinolyl, each of which can be optionally substituted by one or more groups, which may be the same or different, selected from halogen, hydroxy, cyano, C₁-C₈ alkoxy, CO₂R⁸, CONR⁹R¹⁰, C₁-C₈ alkyl-NR⁸—C₁-C₈ alkyl, C₁-C₈ alkyl-CONR⁸—C₁-C₈ alkyl, C₁-C₈ alkyl-CONR⁹R¹⁰, NR⁸COC₁-C₈ alkyl, C₁-C₈ thioalkyl, C₁-C₈ alkyl (itself optionally substituted by one or more hydroxy, azido or cyano groups or fluorine atoms), C₁-C₈ alkyl-NR¹¹R¹², C₁-C₈ alkyl-OR¹², C₁-C₈ alkyl-SR¹²;
R⁸ is hydrogen or C₁-C₈ alkyl;
R⁹ and R¹⁰ are each independently hydrogen or C₁-C₈ alkyl
R¹¹ is hydrogen or C₁-C₈ alkyl;
R¹² is hydrogen or a group selected from C₁-C₈ alkyl, —(CR¹³₂)ₙR¹⁴, —CO—(CR¹³₂)ₙR¹⁴, —SO₂—(CR¹³₂)ₙR¹⁴;
n is between 0 and 5;
R¹³ groups are independently hydrogen, C₁-C₈ alkyl, hydroxy, C₁-C₈ alkoxy, hydroxy(C₁-C₈)alkyl, amino or halogen;
R¹⁴ is hydrogen or a group selected from —NR¹⁵R¹⁶, C₁-C₈ alkyl, C₂-C₄ alkenyl, C₂-C₄ alkynyl, —COOH, —S(C₁-C₈ alkyl), —SO(C₁-C₈ alkyl), —CONR¹⁵R¹⁶, —CO(C₁-C₈ alkyl), —CO—O—(C₁-C₈ alkyl), or a saturated or unsaturated 4- to 10-membered ring, which ring may optionally comprise one or more heteroatoms selected from nitrogen, oxygen and sulphur, each of which groups may be optionally substituted by one or more hydroxy, C₁-C₈ alkyl (which may itself optionally be substituted by a 4- to 7-membered saturated or unsaturated heterocyclic ring system optionally containing a further oxygen, sulphur or nitrogen atom, the ring being optionally substituted by one or more hydroxy, hydroxy(C₁-C₈)alkyl, C₁-C₈ alkyl, nitro, —CONH₂ groups), C₁-C₈ alkoxy, C₁-C₈ hydroxyalkyl, —C═O, cyano, amino, nitro, halogen, C₁-C₈ alkylsulphonyl or aminosulphonyl groups or by a saturated monocyclic 4- to 7-membered ring, which ring may optionally comprise one or more heteroatoms selected from nitrogen, oxygen and sulphur;
or R¹¹ and R¹², together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl (which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—; and
R¹⁵ and R¹⁶, which may be the same or different, represent hydrogen, C₁-C₈ alkyl, —CONH₂ or —C(NH₂)═NH;
provided that when
R^{x} is Ar, X is —CO and R¹ and R² are independently nitro, cyano, C₁-C₈ alkyl, C₁-C₈ alkoxy, hydroxyl, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pR⁶, —CH₂(CH₂)ₚOCOR⁶ or R¹ and R² are linked together as —OCH₂O— or —OCH₂CH₂O—,
where each R³ group is independently hydrogen, C₁-C₈ alkyl, hydroxy, or halogen,
R⁴ and R⁵ each independently represent hydrogen or C₁-C₈ alkyl or R⁴ and R⁵ together with the nitrogen atom to which they are attached form an unsubstituted 4- to 7-membered saturated or aromatic heterocyclic ring system optionally containing a further oxygen, sulphur or NR⁶ group or one of R⁴ and R⁵ is hydrogen or C₁-C₈ alkyl and the other is a 5- or 6-membered heterocyclic ring system optionally containing a further oxygen, sulphur or nitrogen atom;
and R⁶ is selected from hydrogen, (C₁-C₈)alkyl, —CO(C₁-C₈)alkyl, hydroxy substituted (C₁-C₈)alkyl, halogen substituted (C₁-C₈)alkyl, phenyl or benzyl,
then
Ar is selected from dihydroisoquinolyl, oxodihydroisoquinolyl, tetrahydroisoquinolyl or oxotetrahydroisoquinolyl, each of which may be optionally substituted,
or Ar is phenyl substituted by at least one substituent selected from azido substituted C₁-C₈ alkyl, C₁-C₈ alkyl-NR¹¹R¹², C₁-C₈ alkyl-OR¹² or C₁-C₈ alkyl-SR¹²,
wherein R¹² is selected from —(CR¹³₂)ₙR¹⁴, —CO—(CR¹³₂)ₙR¹⁴, —SO₂—(CR¹³₂)ₙR¹⁴ or R¹¹ and R¹², together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl (which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—,
provided that Ar is not phenyl substituted by one or more groups selected from C₁-C₈ alkyl-NR¹¹—C₁-C₈ alkyl, C₁-C₈ alkyl-O—C₁-C₈ alkyl or C₁-C₆ alkanoyloxy C₁-C₆ alkyl

Unless otherwise indicated, the term ‘alkyl’ when used alone or in combination, refers to a straight chain or branched chain alkyl moiety. A C₁-C₈ alkyl group has from one to eight carbon atoms including methyl, ethyl, n-propyl, isopropyl, tert-butyl, n-pentyl, n-hexyl and the like. References to individual alkyl groups such as “propyl” are specific for the straight-chain version only, references to individual branched-chain alkyl groups such as “isopropyl” are specific for the branched-chain version only.

Analogously, the term ‘C₁-C₈ alkoxy’, when used alone or in combination, will be understood to refer to straight or branched chain groups having from one to eight or from one to four carbon atoms respectively and includes such groups as methoxy, ethoxy, propoxy, isopropoxy and butoxy.

The term ‘cycloalkyl’, when used alone or in combination, refers to a saturated alicyclic moiety having from three to eight carbon atoms and includes, for example, cyclopropyl, cyclobutyl, cyclopentyl, cyclohexyl and cycloheptyl.

The term aryl includes phenyl and naphthyl groups.

A C₂-C₄ alkenyl group is for example vinyl or allyl. A C₂-C₄ alkynyl group is for example ethynyl or propyn-2-yl.

‘Optionally substituted’ is used herein to indicate optional substitution by the group or groups specified at any suitable available position.

A ‘heteroatom’ is a nitrogen, sulphur or oxygen atom. Where rings include nitrogen atoms, these may be substituted as necessary to fulfil the bonding requirements of nitrogen or they may be linked to the rest of the structure by way of the nitrogen atom. Nitrogen atoms may also be in the form of N-oxides. Sulphur atoms may be in the form of S, S(O) or SO₂. In a heterocyclic ring, a —CH₂— group can optionally be replaced by a —C(O).

As used herein, the term ‘halogen’ includes fluorine, chlorine, bromine and iodine.

A ‘saturated or unsaturated 4- to 10-membered ring, which ring may optionally comprise one or more heteroatoms selected from nitrogen, oxygen and sulphur’ may be a saturated, partially saturated or unsaturated monocyclic or bicyclic ring. The ring may be a carbocylic (that is an alicyclic ring having ring carbon atoms only) or is a heterocyclic ring containing four to ten atoms of which at least one is a heteroatom selected from nitrogen, oxygen and sulphur and which ring may, unless otherwise specified, be carbon or nitrogen linked. Examples of suitable carbocyclic rings include cyclobutyl, cyclopentyl, cyclohexyl and cycloheptyl. Suitably a 4- to 10-membered heterocyclic ring may be pyridyl, imidazolyl, isoxazolyl, pyrazolyl, furyl, pyrazinyl, pyridazinyl, pyrimidinyl, pyrrolyl, thiazolyl, oxazolyl, isothiazolyl, triazolyl, tetrazolyl, thienyl, pyrrolidinyl, piperidinyl, thiomorpholinyl, morpholinyl, tetrahydrofuranyl, piperazinyl, imidazopyrrole, indole, isoindole, indoline, isoindazole, benzimidazole, purine, quinolyl (for example, 1,2-dihydroquinolinyl or 1,2,3,4-tetrahydroquinolinyl), isoquinolyl, cinnolinyl, quinazolinyl, quinoxalinyl, benzoxazole, benzothiazole, imidazopyridinyl, imidazopyrimidinyl, imidazopyrazinyl. A saturated monocyclic 4- to 7-membered ring comprising one or more heteroatoms selected from nitrogen, oxygen and sulphur will accordingly be understood to mean a heterocyclic ring containing four to seven atoms and which may, unless otherwise specified, be carbon or nitrogen linked. Particular examples of such ring systems include pyrrolidinyl and piperidinyl as included in the above list.

A ‘4- to 7-membered heteroaromatic ring comprising at least one ring heteroatom selected from nitrogen, oxygen and sulphur’ is a fully unsaturated, aromatic monocyclic ring containing from four to seven atoms of which at least one is a heteroatom selected from nitrogen, oxygen and sulphur, which ring may, unless otherwise specified, be carbon or nitrogen linked. Particular examples of such ring systems include pyridyl, imidazolyl, isoxazolyl, pyrazolyl, furyl, pyrazinyl, pyridazinyl, pyrimidinyl, pyrrolyl, thiazolyl, oxazolyl, isothiazolyl, triazolyl, tetrazolyl or thienyl as given above.

It will be appreciated that the number and nature of substituents on rings in the compounds of the invention will be selected so as to avoid sterically undesirable combinations.

In one particular embodiment, the invention provides a compound of formula (Ia)

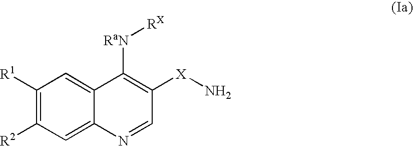

or a pharmaceutically acceptable salt or solvate thereof, wherein

### X is —CHOH or —C═O;

one of R¹ and R² represents nitro, cyano, C₁-C₈ alkyl, C₁-C₈ alkoxy, hydroxy, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pR⁶, Y(CR³₂)ₚOCOR⁶
or R¹ and R² are linked together as —OCH₂O— or —OCH₂CH₂O—;
R³ groups are independently hydrogen, C₁-C₈ alkyl, hydroxy, C₁-C₈ alkoxy or halogen;
p is 0, 1, 2, 3, 4 or 5;
Y is oxygen, CH_{2′}—OSO₂— or NR⁷
R⁴ and R⁵ each independently represent hydrogen or a group selected from C₁-C₈ alkyl, C₁-C₈ alkoxy, —CO—(C₁-C₈)alkyl, —CO—(C₁-C₈)cycloalkyl, —SO₂—(C₁-C₈)alkyl, —CO—(C₁-C₈)alkoxy, —CO—NR⁷(C₁-C₈)alkyl, C₃-C₈ cycloalkyl, each of which groups may optionally be substituted by one or more hydroxy, cyano, —CONH₂ or —CO—(C₁-C₈)alkoxy groups,
or R⁴ and R⁵ together with the nitrogen atom to which they are attached form a 4- to 7-membered, saturated or aromatic heterocyclic ring system optionally containing one or more additional heteroatoms selected from oxygen, sulphur or nitrogen, the ring itself being optionally substituted by at least one substituent selected from hydroxy, C₁-C₈ alkyl, ═O, C₁-C₈ alkoxy or (C₁-C₈ alkoxy)-CO—, or one of R⁴ and R⁵ is hydrogen or C₁-C₈ alkyl and the other is a 5- or 6-membered heterocyclic ring system optionally containing a further oxygen, sulphur or nitrogen atom;
R⁶ is hydrogen, C₁-C₈ alkyl (itself optionally substituted by one or more hydroxy, cyano, halogen or amino groups), phenyl, benzyl, —CO(C₁-C₈)alkyl or a saturated monocyclic 4- to 7-membered ring, which ring may optionally comprise one or more heteroatoms selected from nitrogen, oxygen and sulphur, the ring itself being optionally substituted by at least one substituent selected from C₁-C₈ alkyl, C₁-C₈ alkoxy, ═O, C₁-C₈ alkyl —CO—, or (C₁-C₈ alkoxy)-CO— where any C₁-C₈ alkyl is optionally substituted by one or more hydroxy, cyano, halogen or amino groups;
R⁷ is hydrogen or C₁-C₈ alkyl;
R^{a} is hydrogen or C₁-C₈ alkyl;
R^{x} is a group selected from C₁-C₈ alkyl, C₃-C₈ cycloalkyl or a saturated monocyclic 4- to 7-membered ring comprising one or more heteroatoms selected from nitrogen, oxygen and sulphur, wherein any C₃-C₈ cycloalkyl group or saturated monocyclic 4- to 7-membered ring is optionally substituted by one or more groups selected from hydroxy, azido, cyano, amino, halogen, —CONH₂—, C₁-C₈ alkyl, (C₁-C₈ alkyl)CO—, C₁-C₈ alkoxy, or (C₁-C₈ alkoxy)-CO—, and any C₁-C₈ alkyl, C₁-C₈ alkyl)CO—, C₁-C₈ alkoxy, or (C₁-C₈ alkoxy)-CO— group is itself optionally substituted by one or more substituents selected from hydroxy, azido, cyano, amino, halogen or phenyl; or R^{x} is a group Ar;
Ar is selected from phenyl, tetrahydronaphthenyl, indolyl, pyrazolyl, dihydroindenyl, 1-oxo-2,3-dihydroindenyl, indazolyl, dihydroisoquinolyl, oxodihydroisoquinolyl, tetrahydroisoquinolyl or oxotetrahydroisoquinolyl, each of which can be optionally substituted by one or more groups, which may be the same or different, selected from halogen, hydroxy, cyano, C₁-C₈ alkoxy, CO₂R⁸, CONR⁹R¹⁰, C₁-C₈ alkyl-NR⁸—C₁-C₈ alkyl, C₁-C₈ alkyl-CONR⁸—C₁-C₈ alkyl, C₁-C₈ alkyl-CONR⁹R¹⁰, NR⁸COC₁-C₈ alkyl, C₁-C₈ thioalkyl, C₁-C₈ alkyl (itself optionally substituted by one or more hydroxy, azido or cyano groups or fluorine atoms), C₁-C₈alkyl-NR¹¹R¹², C₁-C₈alkyl-OR¹², C₁-C₈alkyl-SR¹²;
R⁸ is hydrogen or C₁-C₈ alkyl;
R⁹ and R¹⁰ are each independently hydrogen or C₁-C₈ alkyl
R¹¹ is hydrogen or C₁-C₈ alkyl;
R¹² is hydrogen or a group selected from C₁-C₈ alkyl, —(CR¹³₂)ₙR¹⁴, —CO—(CR¹³₂)ₙR¹⁴, —SO₂—(CR¹³₂)ₙR¹⁴;
n is between 0 and 5;
R¹³ groups are independently hydrogen, C₁-C₈ alkyl, hydroxy, C₁-C₈ alkoxy, hydroxy(C₁-C₈)alkyl, amino or halogen;
R¹⁴ is hydrogen or a group selected from —NR¹⁵R¹⁶, C₁-C₈ alkyl, C₂-C₄ alkenyl, C₂-C₄ alkynyl, —COOH, —S(C₁-C₈ alkyl), —SO(C₁-C₈ alkyl), —CONR¹⁵R¹⁶, —CO(C₁-C₈ alkyl), —CO—O—(C₁-C₈ alkyl), or a saturated or unsaturated 4- to 10-membered ring, which ring may optionally comprise one or more heteroatoms selected from nitrogen, oxygen and sulphur, each of which groups may be optionally substituted by one or more hydroxy, C₁-C₈ alkyl (which may itself optionally be substituted by a 4- to 7-membered saturated or unsaturated heterocyclic ring system optionally containing a further oxygen, sulphur or nitrogen atom, the ring being optionally substituted by one or more hydroxy, hydroxy(C₁-C₈)alkyl, C₁-C₈ alkyl, nitro, —CONH₂ groups), C₁-C₈ alkoxy, C₁-C₈ hydroxyalkyl, —C═O, cyano, amino, nitro, halogen, C₁-C₈ alkylsulphonyl or aminosulphonyl groups or by a saturated monocyclic 4- to 7-membered ring, which ring may optionally comprise one or more heteroatoms selected from nitrogen, oxygen and sulphur;
or R¹¹ and R¹², together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, hydroxy(C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—; and
R¹⁵ and R¹⁶, which may be the same or different, represent hydrogen, C₁-C₈ alkyl, —CONH₂ or —C(NH₂)═NH;
and the other of R¹ and R² is Y(CR³₂)ₚNR⁴R⁵, Y(CR³₂)ₙCONR⁴R⁵, Y(CR³₂)ₚ—C₂R⁶, Y(CR³₂)ₚOR⁶, Y(CR³₂)pR⁶ or Y(CR³₂)ₚOCOR⁶,
where at least one R³ is C₁-C₈ alkoxy, or one of R⁴ and R⁵ is selected from optionally substituted —CO—(C₁-C₈)alkyl, —CO—(C₁-C₈)cycloalkyl, —SO₂—(C₁-C₈)alkyl, —CO—(C₁-C₈)alkoxy, —CO—NR⁷(C₁-C₈)alkyl or C₃-C₈ cycloalkyl, or R⁴ and R⁵ together with the nitrogen atom to which they are attached form a substituted 4- to 7-membered saturated or aromatic heterocyclic ring system optionally containing a further oxygen, sulphur or NR⁶ group, or R⁶ is selected from —CO(C₁-C₈)alkyl, or an optionally substituted saturated monocyclic 4- to 7-membered ring, which ring may optionally comprise one or more heteroatoms selected from nitrogen, oxygen and sulphur, and which may be optionally substituted by at least one substituent selected from C₁-C₈ alkyl, C₁-C₈ alkoxy, C₁-C₈ alkyl —CO—, ═O or (C₁-C₈ alkoxy)-CO— where any C₁-C₈ alkyl is optionally substituted by one or more hydroxy, cyano, halogen or amino groups;

In another particular embodiment, the invention provides a compound of formula (Ib)

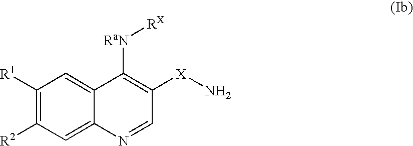

or a pharmaceutically acceptable salt or solvate thereof, wherein

### X is —CHOH or —C═O;

R¹ and R², which may be the same or different, represent nitro, cyano, C₁-C₈ alkyl, C₁-C₈ alkoxy, hydroxy, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pR⁶, Y(CR³₂)ₚOCOR⁶
or R¹ and R² are linked together as —OCH₂O— or —OCH₂CH₂O—;
R³ groups are independently hydrogen, C₁-C₈ alkyl, hydroxy, C₁-C₈ alkoxy or halogen;
p is 0, 1, 2, 3, 4 or 5;
Y is oxygen, CH_{2′}—OSO₂— or NR⁷
R⁴ and R⁵ each independently represent hydrogen or a group selected from C₁-C₈ alkyl, C₁-C₈ alkoxy, —CO—(C₁-C₈)alkyl, —CO—(C₁-C₈)cycloalkyl, —SO₂—(C₁-C₈)alkyl, —CO—(C₁-C₈)alkoxy, —CO—NR⁷(C₁-C₈)alkyl, C₃-C₈ cycloalkyl, each of which groups may optionally be substituted by one or more hydroxy, cyano, —CONH₂ or —CO—(C₁-C₈)alkoxy groups,
or R⁴ and R⁵ together with the nitrogen atom to which they are attached form a 4- to 7-membered, saturated or aromatic heterocyclic ring system optionally containing one or more additional heteroatoms selected from oxygen, sulphur or nitrogen, the ring itself being optionally substituted by at least one substituent selected from hydroxy, C₁-C₈ alkyl, ═O, C₁-C₈ alkoxy or (C₁-C₈ alkoxy)-CO—, or one of R⁴ and R⁵ is hydrogen or C₁-C₈ alkyl and the other is a 5- or 6-membered heterocyclic ring system optionally containing a further oxygen, sulphur or nitrogen atom;
R⁶ is hydrogen, C₁-C₈ alkyl (itself optionally substituted by one or more hydroxy, cyano, halogen or amino groups), phenyl, benzyl, —CO(C₁-C₈)alkyl or a saturated monocyclic 4- to 7-membered ring, which ring may optionally comprise one or more heteroatoms selected from nitrogen, oxygen and sulphur, the ring itself being optionally substituted by at least one substituent selected from C₁-C₈ alkyl, C₁-C₈ alkoxy, ═O or (C₁-C₈ alkoxy)-CO—; C₁-C₈ alkyl —CO—, where any C₁-C₈ alkyl is optionally substituted by one or more hydroxy, cyano, halogen or amino groups;
R⁷ is hydrogen or C₁-C₈ alkyl;
R^{a} is hydrogen or C₁-C₈ alkyl;
R^{x} is a group selected from C₁-C₈ alkyl, C₃-C₈ cycloalkyl or a saturated monocyclic 4- to 7-membered ring comprising one or more heteroatoms selected from nitrogen, oxygen and sulphur, wherein any C₃-C₈ cycloalkyl group or saturated monocyclic 4- to 7-membered ring is optionally substituted by one or more groups selected from hydroxy, azido, cyano, amino, halogen, —CONH₂—, C₁-C₈ alkyl, (C₁-C₈ alkyl)CO—, C₁-C₈ alkoxy, or (C₁-C₈ alkoxy)-CO—, and any C₁-C₈ alkyl, C₁-C₈ alkyl)CO—, C₁-C₈ alkoxy, or (C₁-C₈ alkoxy)-CO— group is itself optionally substituted by one or more substituents selected from hydroxy, azido, cyano, amino, halogen or phenyl; or R^{x} is a group Ar;
Ar is selected from dihydroisoquinolyl, oxodihydroisoquinolyl, tetrahydroisoquinolyl or oxotetrahydroisoquinolyl, each of which can be optionally substituted by one or more groups, which may be the same or different, selected from halogen, hydroxy, cyano, C₁-C₈ alkoxy, CO₂R⁸, CONR⁹R¹⁰, C₁-C₈ alkyl-NR⁸—C₁-C₈ alkyl, C₁-C₈ alkyl-CONR⁸—C₁-C₈ alkyl, C₁-C₈ alkyl-CONR⁹R¹⁰, NR⁸COC₁-C₈ alkyl, C₁-C₈ thioalkyl, C₁-C₈ alkyl (itself optionally substituted by one or more hydroxy, azido or cyano groups or fluorine atoms), C₁-C₈ alkyl-NR¹¹R¹², C₁-C₈ alkyl-OR¹², C₁-C₈ alkyl-SR¹²;
or Ar is phenyl substituted by at least one substituent selected from azido substituted C₁-C₈ alkyl, C₁-C₈ alkyl-NR¹¹R^{12a}, C₁-C₈ alkyl-OR^{12a}, C₁-C₈ alkyl-SR^{12a},
wherein R^{12a} is selected from —(CR¹³₂)ₙR¹⁴, —CO—(CR¹³₂)ₙR¹⁴, —SO₂—(CR¹³₂)ₙR¹⁴;
R⁸ is hydrogen or C₁-C₈ alkyl;
R⁹ and R¹⁰ are each independently hydrogen or C₁-C₈ alkyl
R¹¹ is hydrogen or C₁-C₈ alkyl;
R¹² is hydrogen or a group selected from C₁-C₈ alkyl, —(CR¹³₂)ₙR¹⁴, —CO—(CR¹³₂)ₙR¹⁴, —SO₂—(CR³₂)ₙR¹⁴;
n is between 0 and 5;
R¹³ groups are independently hydrogen, C₁-C₈ alkyl, hydroxy, C₁-C₈ alkoxy, hydroxy(C₁-C₈)alkyl, amino or halogen;
R¹⁴ is hydrogen or a group selected from —NR¹⁵R¹⁶, C₁-C₈ alkyl, C₂-C₄ alkenyl, C₂-C₄ alkynyl, —COOH, —S(C₁-C₈ alkyl), —SO(C₁-C₈ alkyl), —CONR¹⁵R¹⁶, —CO(C₁-C₈ alkyl), —CO—O—(C₁-C₈ alkyl), or a saturated or unsaturated 4- to 10-membered ring, which ring may optionally comprise one or more heteroatoms selected from nitrogen, oxygen and sulphur, each of which groups may be optionally substituted by one or more hydroxy, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, hydroxy(C₁-C₈)alkyl, C₁-C₈ alkyl, nitro, —CONH₂ groups), C₁-C₈ alkoxy, C₁-C₈ hydroxyalkyl, —C═O, cyano, amino, nitro, halogen, C₁-C₈ alkylsulphonyl or aminosulphonyl groups or by a saturated monocyclic 4- to 7-membered ring, which ring may optionally comprise one or more heteroatoms selected from nitrogen, oxygen and sulphur;
or R¹¹ and R¹², together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl (which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—; and
R¹⁵ and R¹⁶, which may be the same or different, represent hydrogen, C₁-C₈ alkyl —CONH₂ or —C(NH₂)═NH,
provided that Ar is not phenyl substituted by one or more groups selected from C₁-C₈ alkyl-NR¹¹—C₁-C₈ alkyl, C₁-C₈ alkyl-O—C₁-C₈ alkyl or C₁-C₆ alkanoyloxy C₁-C₆ alkyl.

Suitably X is —CHOH or —C═O, preferably —C═O.

In one embodiment, R^{x} is a group selected from C₁-C₈ alkyl, C₃-C₈ cycloalkyl or a saturated monocyclic 4- to 7-membered ring comprising one or more heteroatoms selected from nitrogen, oxygen and sulphur, wherein any C₃-C₈ cycloalkyl group or saturated monocyclic 4- to 7-membered ring is optionally substituted by one or more groups selected from hydroxy, azido, cyano, amino, halogen, —CONH₂—, C₁-C₈ alkyl, (C₁-C₈ alkyl)CO—, C₁-C₈ alkoxy, or (C₁-C₈ alkoxy)-CO—, and any C₁-C₈ alkyl, (C₁-C₈ alkyl)CO—, C₁-C₈ alkoxy, or (C₁-C₈ alkoxy)-CO— group is itself optionally substituted by one or more substituents selected from hydroxy, azido, cyano, amino, halogen or phenyl.

In one preferred embodiment, R^{x} is C₃-C₈ cycloalkyl or a saturated monocyclic 4- to 7-membered ring comprising one or more heteroatoms selected from nitrogen, oxygen and sulphur, each of which groups is optionally substituted as described above. Preferably R^{x} is cyclohexyl, pyrrolidinyl or piperidinyl, optionally substituted as described above.

Substituents may be present on any suitable position of the R^{x} group and more than one substitutent, which may be the same or different, may be present. Where R^{x} is substituted, this is preferably by one or two substituents.

Preferred substituents on R^{x} include C₁-C₈ alkyl, (C₁-C₈ alkyl)CO—, C₁-C₈ alkoxy, or (C₁-C₈ alkoxy)-CO—, optionally substituted with one or more substituents selected from hydroxy, azido, cyano, amino, halogen, —CONH₂, C₁-C₈ alkoxy, (C₁-C₈ alkoxy)-CO— or phenyl.

Particularly preferred substituents on R^{x} include methyl, ethyl, benzyl, (CH₃)C—O—CO—, —COCN.

In another embodiment, R^{x} is a group Ar.

Suitably Ar is selected from phenyl, tetrahydronaphthenyl, indolyl, pyrazolyl, dihydroindenyl, 1-oxo-2,3-dihydroindenyl or indazolyl optionally substituted as described above. Substituents can be present on any suitable position of the Ar group. More than one substituent can be present, and these can be the same or different. Preferably Ar is optionally substituted dihydroisoquinolyl, oxodihydroisoquinolyl, tetrahydroisoquinolyl, oxotetrahydroisoquinolyl or phenyl, most preferably phenyl.

Where Ar is phenyl, this is preferably substituted by one and especially two substitutents. Preferred substituents include C₁-C₈ alkyl, such as methyl or ethyl, hydroxy(C₁-C₈)alkyl, for example hydroxymethyl or hydroxyethyl, or a C₁-C₈ alkyl-NR¹¹R¹², C₁-C₈ alkyl-OR¹², C₁-C₈ alkyl-SR¹² group such as CH₂SR¹², CH₂OR¹² or especially —CH₂NR¹¹R¹².

In one preferred embodiment, Ar is phenyl substituted by at least one substituent selected C₁-C₈ alkyl-NR¹¹R^{12a}, C₁-C₈, alkyl-OR^{12a}, C₁-C₈ alkyl-SR^{12a},

wherein R^{12a} is selected from —(CR¹³₂)ₙR¹⁴, —CO—(CR¹³₂)ₙR¹⁴, —SO₂—(CR¹³₂)ₙR¹⁴;
provided that Ar is not phenyl substituted by one or more groups selected from C₁-C₈ alkyl-NR¹¹—C₁-C₈ alkyl, C₁-C₈ alkyl-O—C₁-C₈ alkyl or C₁-C₆ alkanoyloxy C₁-C₆ alkyl.

In one embodiment, R¹¹ is preferably hydrogen

In another embodiment, R¹² is preferably a group —(CR¹³₂)ₙR¹⁴

R¹³ is preferably hydrogen

R¹⁴ may be hydrogen, —NR¹⁵R¹⁶ or C₁-C₈ alkyl but is preferably a saturated or unsaturated 4- to 10-membered ring, which ring may optionally comprise one or more heteroatoms selected from nitrogen, oxygen and sulphur, each of which groups may be optionally substituted by one or more hydroxy, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), C₁-C₈ alkoxy, C₁-C₈ hydroxyalkyl, —C═O, cyano, amino, nitro, halogen, C₁-C₈ alkylsulphonyl or aminosulphonyl groups or by a saturated monocyclic 4- to 7-membered ring, which ring may optionally comprise one or more heteroatoms selected from nitrogen, oxygen and sulphur;

In a particular embodiment, R¹¹ and R¹² together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), nitro, cyano, —CONH₂, amino or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—;

R^{a} is hydrogen or C₁-C₈ alkyl such as methyl or ethyl. Where R^{a} is C₁-C₈ alkyl, it is preferably methyl.

In one embodiment, R^{a} is C₁-C₈ alkyl, especially methyl.

In a particular embodiment, R^{a} is hydrogen.

Suitably R¹ and R² are independently selected from hydrogen, halogen, nitro, cyano, C₁-C₈ alkyl, C₁-C₈ alkoxy, hydroxy, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pR⁶, Y(CR³₂)ₚOCOR⁶; or R¹ and R² are linked together as —OCH₂O— or —OCH₂CH₂O—.

In one embodiment, R¹ and R² independently preferably represent C₁-C₈ alkoxy, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)ₚOCOR⁶, Y(CR³₂)pR⁶.

In one embodiment, one or both of R¹ and R² is Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pR⁶ or Y(CR³₂)ₚOCOR⁶, wherein at least one R³ is alkoxy, or one of R⁴ and R⁵ is a group selected from —CO—(C₁-C₈)alkyl, —CO—(C₁-C₈)cycloalkyl, —SO₂—(C₁-C₈)alkyl, —CO—(C₁-C₈)alkoxy, —CO—NR⁷(C₁-C₈)alkyl or C₃-C₈ cycloalkyl, each of which groups may optionally be substituted by one or more hydroxy, cyano, —CONH₂ or —CO—(C₁-C₈)alkoxy groups, or R⁴ and R⁵ together with the nitrogen atom to which they are attached form a 4- to 7-membered saturated or aromatic heterocyclic ring system optionally containing one or more additional heteroatoms selected from oxygen, sulphur or nitrogen, which ring system is substituted by at least one substituent selected from hydroxy, C₁-C₈ alkyl, ═O, C₁-C₈ alkoxy or (C₁-C₈ alkoxy)-CO—, or R⁶ is selected from —CO(C₁-C₈)alkyl or a saturated monocyclic 4- to 7-membered ring, which ring may optionally comprise one or more heteroatoms selected from nitrogen, oxygen and sulphur, the ring itself being optionally substituted by at least one substituent selected from C₁-C₈ alkyl, C₁-C₈ alkoxy or (C₁-C₈ alkoxy)-CO—.

In a further embodiment, R¹ and R² independently preferably represent methoxy, ethoxy, —O(CH₂)₂NR⁴R⁵, O(CH₂)₃ NR⁴R⁵, —OR⁶, —O(CH₂)₂R⁶, —N(CR³)₂NR⁴R⁵, —N(CR³)₃NR⁴R⁵, —N(CR³)₂OR⁶, —N(CR³)₃OR⁶.

Each R³ group independently may suitably represent hydrogen, C₁-C₈ alkyl, hydroxy, C₁-C₈ alkoxy or halogen but preferably each R³ independently represents hydrogen or C₁-C₈ alkoxy such as methoxy or ethoxy.

R⁴ and R⁵ each independently preferably represent hydrogen or a group selected from C₁-C₈ alkyl, —CO—(C₁-C₈)alkyl, —SO₂—(C₁-C₈)alkyl, C₃-C₈ cycloalkyl, each of which groups may be optionally substituted as described above, or R⁴ and R⁵ together with the nitrogen atom to which they are attached form a 4- to 7-membered, substituted or unsubstituted, saturated or aromatic heterocyclic ring system optionally containing a further oxygen, sulphur or NR⁶ group. Particularly preferably, R⁴ and R⁵ each independently represent hydrogen, —CH₃, —(CH₂)₂CN, —COCH₃—COCH(CH₃)₂, —CH(CH₃)₂, cyclopropyl, —CO-cyclopropyl, —SO₂CH₃, —C(═O)—O—C(CH₃)₃, or R⁴ and R⁵ together represent an optionally substituted piperidinyl, pyrrolidinyl, piperazinyl, 1,2,4-triazolyl, 2,5-dioxopyrrolidinyl or 2,5-dioxoimidazolidinyl group.

In a particular embodiment, R¹ and R² are both C₁-C₈ alkoxy, or one of R¹ and R² is C₁-C₈ alkoxy and the other is Y(CR³₂)ₙNR⁴R⁵, Y(CR³₂)ₚCONR⁴R⁵, Y(CR³₂)ₚCO₂R⁶, Y(CR⁶₂)ₚOR⁶, Y(CR³₂)pR⁶ or Y(CR³₂)ₚOCOR⁶.

Where R¹ and R² are both C₁-C₈ alkoxy, this is preferably methoxy or ethoxy. In one particular embodiment, R¹ and R² are both methoxy or ethoxy.

Preferred compounds of the invention include:—
6,7-diethoxy-4-{[2-ethyl-3-(1H-imidazol-1-ylmethyl)phenyl]amino}quinoline-3-carboxamide
6,7-diethoxy-4-{[2-methyl-3-(1H-1,2,4-triazol-1-ylmethyl)phenyl]amino}quinoline-3-carboxamide
6,7-diethoxy-4-{[2-ethyl-3-(morpholin-4-ylmethyl)phenyl]amino}quinoline-3-carboxamide
6,7-diethoxy-4-{[3-(1H-imidazol-1-ylmethyl)-2-methylphenyl]amino}quinoline-3-carboxamide
4-{[3-(azidomethyl)-2-methylphenyl]amino}-6,7-diethoxyquinoline-3-carboxamide
6,7-diethoxy-4-{[2-methyl-3-(4H-1,2,4-triazol-4-ylmethyl)phenyl]amino}quinoline-3-carboxamide
4-{[3-({[4-(aminosulfonyl)benzyl]amino}methyl)-2-ethylphenyl]amino}-6,7-dimethoxyquinoline-3-carboxamide
4-({2-ethyl-3-[(1H-1,2,4-triazol-5-ylamino)methyl]phenyl}amino)-6,7-dimethoxyquinoline-3-carboxamide
4-{[2-ethyl-3-(1H-imidazol-1-ylmethyl)phenyl]amino}-6,7-dimethoxyquinoline-3-carboxamide
6,7-diethoxy-4-({2-ethyl-3-[(pyrimidin-2-ylamino)methyl]phenyl}amino)quinoline-3-carboxamide
6,7-diethoxy-4-[(2-ethyl-3-{[(2-hydroxycyclohexyl)amino]methyl}phenyl)amino]quinoline-3-carboxamide
6,7-diethoxy-4-[(2-ethyl-3-{[(3-thienylmethyl)amino]methyl}phenyl)amino]quinoline-3-carboxamide
6,7-diethoxy-4-({2-ethyl-3-[(1H-imidazol-2-ylthio)methyl]phenyl}amino)quinoline-3-carboxamide
6,7-diethoxy-4-{[2-ethyl-3-(thiomorpholin-4-ylmethyl)phenyl]amino}quinoline-3-carboxamide
6,7-diethoxy-4-[(2-ethyl-3-{[(3-thienylmethyl)amino]methyl}phenyl)amino]quinoline-3-carboxamide
4-({2-ethyl-3-[(4-nitro-1H-imidazol-1-yl)methyl]phenyl}amino)-6,7-dimethoxyquinoline-3-carboxamide
4-[(2-ethyl-3-{[4-(hydroxymethyl)-1H-imidazol-1-yl]methyl}phenyl)amino]-6,7-dimethoxyquinoline-3-carboxamide trifluoroacetate (salt)
4-({2-ethyl-3-[(2-methyl-1H-imidazol-1-yl)methyl]phenyl}amino)-6,7-dimethoxyquinoline-3-carboxamide
1-(3-{[3-(aminocarbonyl)-6,7-dimethoxyquinolin-4-yl]amino}-2-ethylbenzyl)-1H-imidazole-4-carboxylic acid
4-({3-[(cyclopentylamino)methyl]-2-ethylphenyl}amino)-6,7-dimethoxyquinoline-3-carboxamide
4-{[2-ethyl-3-({[2-(1H-imidazol-4-yl)ethyl]amino}methyl)phenyl]amino}-6,7-dimethoxyquinoline-3-carboxamide
4-[(2-ethyl-3-{[(2-hydroxy-1,1-dimethylethyl)amino]methyl}phenyl)amino]-6,7-dimethoxyquinoline-3-carboxamide
4-({2-ethyl-3-[(1,3-thiazol-2-ylamino)methyl]phenyl}amino)-6,7-dimethoxyquinoline-3-carboxamide
4-[(2-ethyl-3-{[(2-hydroxypropyl)amino]methyl}phenyl)amino]-6,7-dimethoxyquinoline-3-carboxamide
4-[(2-ethyl-3-{[(2-hydroxy-2-phenylethyl)amino]methyl}phenyl)amino]-6,7-dimethoxyquinoline-3-carboxamide bis(trifluoroacetate)
4-{[2-ethyl-3-({[4-(methylsulfonyl)benzyl]amino}methyl)phenyl]amino}-6,7-dimethoxyquinoline-3-carboxamide
4-({3-[(benzylamino)methyl]-2-ethylphenyl}amino)-6,7-dimethoxyquinoline-3-carboxamide
4-({2-ethyl-3-[(3-methyl-2,5-dioxoimidazolidin-1-yl)methyl]phenyl}amino)-6,7-dimethoxyquinoline-3-carboxamide
4-({2-ethyl-3-[(1H-tetrazol-5-ylamino)methyl]phenyl}amino)-6,7-dimethoxyquinoline-3-carboxamide
4-({3-[(5-amino-1H-tetrazol-1-yl)methyl]-2-ethylphenyl}amino)-6,7-dimethoxyquinoline-3-carboxamide
4-{[2-ethyl-3-({[2-(2-oxoimidazolidin-1-yl)ethyl]amino}methyl)phenyl]amino}-6,7-dimethoxyquinoline-3-carboxamide
4-{[2-ethyl-3-({[(2S)-2-hydroxycyclohexyl]amino}methyl)phenyl]amino}-6,7-dimethoxyquinoline-3-carboxamide
4-({2-ethyl-3-[(piperidin-4-ylamino)methyl]phenyl}amino)-6,7-dimethoxyquinoline-3-carboxamide
4-{[2-ethyl-3-({[(1R)-1-(hydroxymethyl)-3-methylbutyl]amino}methyl)phenyl]amino}-6,7-dimethoxyquinoline-3-carboxamide
6,7-diethoxy-4-[(2-ethyl-3-{[4-(3-methoxyphenyl)piperazin-1-yl]methyl}phenyl)amino]quinoline-3-carboxamide
6,7-diethoxy-4-[(2-ethyl-3-{[4-(hydroxymethyl)piperidin-1-yl]methyl}phenyl)amino]quinoline-3-carboxamide
6,7-diethoxy-4-[(2-ethyl-3-{[2-(hydroxymethyl)piperidin-1-yl]methyl}phenyl)amino]quinoline-3-carboxamide
4-{[3-(1,4′-bipiperidin-1′-ylmethyl)-2-ethylphenyl]amino}-6,7-diethoxyquinoline-3-carboxamide
4-[(3-{[4-(aminocarbonyl)piperidin-1-yl]methyl}-2-ethylphenyl)amino]-6,7-diethoxyquinoline-3-carboxamide
4-[(3-{[4-(2-cyanophenyl)piperazin-1-yl]methyl}-2-ethylphenyl)amino]-6,7-diethoxyquinoline-3-carboxamide
4-[(3-{[4-(5-cyanopyridin-2-yl)piperazin-1-yl]methyl}-2-ethylphenyl)amino]-6,7-diethoxyquinoline-3-carboxamide
6,7-diethoxy-4-[(2-ethyl-3-{[(3-furylmethyl)amino]methyl}phenyl)amino]quinoline-3-carboxamide
6,7-diethoxy-4-[(2-ethyl-3-{[4-(2-hydroxyethyl)piperazin-1-yl]methyl}phenyl)amino]quinoline-3-carboxamide
6,7-diethoxy-4-({2-ethyl-3-[(4-hydroxypiperidin-1-yl)methyl]phenyl}amino)quinoline-3-carboxamide
4-{[3-({[2-(1,3-benzodioxol-5-yl)ethyl]amino}methyl)-2-ethylphenyl]amino}-6,7-diethoxyquinoline-3-carboxamide
6,7-diethoxy-4-{[2-ethyl-3-({[2-(2-thienyl)ethyl]amino}methyl)phenyl]amino}quinoline-3-carboxamide
4-{[3-({[(2,5-dimethyl-3-furyl)methyl]amino}methyl)-2-ethylphenyl]amino}-6,7-diethoxyquinoline-3-carboxamide
6,7-diethoxy-4-{[2-ethyl-3-({[3-(2-oxopyrrolidin-1-yl)propyl]amino}methyl)phenyl]amino}quinoline-3-carboxamide
4-{[3-({[2-(3-chlorophenyl)ethyl]amino}methyl)-2-ethylphenyl]amino}-6,7-diethoxyquinoline-3-carboxamide
4-{[3-({[2-(4-chlorophenyl)ethyl]amino}methyl)-2-ethylphenyl]amino}-6,7-diethoxyquinoline-3-carboxamide
4-{[3-({[2-(2-chlorophenyl)ethyl]amino}methyl)-2-ethylphenyl]amino}-6,7-diethoxyquinoline-3-carboxamide
6,7-diethoxy-4-[(2-ethyl-3-{[(2-hydroxy-2-phenylethyl)amino]methyl}phenyl)amino]quinoline-3-carboxamide
4-({3-[(cyclopentylamino)methyl]-2-ethylphenyl}amino)-6,7-diethoxyquinoline-3-carboxamide
6,7-diethoxy-4-{[2-ethyl-3-({[2-(1H-imidazol-4-yl)ethyl]amino}methyl)phenyl]amino}quinoline-3-carboxamide
6,7-diethoxy-4-[(2-ethyl-3-{[4-(2-morpholin-4-ylethyl)piperazin-1-yl]methyl}phenyl)amino]quinoline-3-carboxamide
4-{[3-({[(2,2-dimethyl-1,3-dioxolan-4-yl)methyl]amino}methyl)-2-ethylphenyl]amino}-6,7-diethoxyquinoline-3-carboxamide
6,7-diethoxy-4-({2-ethyl-3-[(1,3-thiazol-2-ylamino)methyl]phenyl}amino)quinoline-3-carboxamide
6,7-diethoxy-4-{[2-ethyl-3-(1,3-thiazolidin-3-ylmethyl)phenyl]amino}quinoline-3-carboxamide
6,7-diethoxy-4-[(2-ethyl-3-{[(2-pyridin-2-ylethyl)amino]methyl}phenyl)amino]quinoline-3-carboxamide
6,7-diethoxy-4-({2-ethyl-3-[(1H-1,2,4-triazol-3-ylamino)methyl]phenyl}amino)quinoline-3-carboxamide
6,7-diethoxy-4-{[2-ethyl-3-({[4-(2-thienyl)benzyl]amino}methyl)phenyl]amino}quinoline-3-carboxamide
4-{[3-({[4-(aminosulfonyl)benzyl]amino}methyl)-2-ethylphenyl]amino}-6,7-diethoxyquinoline-3-carboxamide
6,7-diethoxy-4-{[2-ethyl-3-({[2-(1H-indol-3-yl)ethyl]amino}methyl)phenyl]amino}quinoline-3-carboxamide
6,7-diethoxy-4-{[2-ethyl-3-({[3-(4-methylpiperazin-1-yl)propyl]amino}methyl)phenyl]amino}quinoline-3-carboxamide
6,7-diethoxy-4-[(2-ethyl-3-{[(1-ethylpiperidin-3-yl)amino]methyl}phenyl)amino]quinoline-3-carboxamide
6,7-diethoxy-4-[(2-ethyl-3-{[4-(pyridin-4-ylmethyl)piperazin-1-yl]methyl}phenyl)amino]quinoline-3-carboxamide
6,7-diethoxy-4-[(2-ethyl-3-{[(pyridin-4-ylmethyl)amino]methyl}phenyl)amino]quinoline-3-carboxamide
6,7-diethoxy-4-[(2-ethyl-3-{[(pyridin-3-ylmethyl)amino]methyl}phenyl)amino]quinoline-3-carboxamide
4-({3-[(benzylamino)methyl]-2-ethylphenyl}amino)-6,7-diethoxyquinoline-3-carboxamide
6,7-diethoxy-4-[(2-ethyl-3-{[(2-furylmethyl)amino]methyl}phenyl)amino]quinoline-3-carboxamide
6,7-diethoxy-4-[(2-ethyl-3-{[(2-methoxyethyl)amino]methyl}phenyl)amino]quinoline-3-carboxamide
6,7-diethoxy-4-[(2-ethyl-3-{[(2-hydroxypropyl)amino]methyl}phenyl)amino]quinoline-3-carboxamide
6,7-diethoxy-4-{[2-ethyl-3-({[4-(1H-pyrazol-1-yl)benzyl]amino}methyl)phenyl]amino}quinoline-3-carboxamide
4-({3-[({2-[4-(aminosulfonyl)phenyl]ethyl}amino)methyl]-2-ethylphenyl}amino)-6,7-diethoxyquinoline-3-carboxamide
6,7-diethoxy-4-{[2-ethyl-3-({[2-(1-methylpyrrolidin-2-yl)ethyl]amino}methyl)phenyl]amino}quinoline-3-carboxamide
4-[(3-{[(4-chlorobenzyl)amino]methyl}-2-ethylphenyl)amino]-6,7-diethoxyquinoline-3-carboxamide
4-[(3-{[(1-benzylpiperidin-4-yl)amino]methyl}-2-ethylphenyl)amino]-6,7-diethoxyquinoline-3-carboxamide
6,7-diethoxy-4-[(2-ethyl-3-{[(3-methoxybenzyl)amino]methyl}phenyl)amino]quinoline-3-carboxamide
6,7-diethoxy-4-[(2-ethyl-3-{[(4-methoxybenzyl)amino]methyl}phenyl)amino]quinoline-3-carboxamide
6,7-diethoxy-4-{[2-ethyl-3-({[3-(1H-imidazol-1-yl)propyl]amino}methyl)phenyl]amino}quinoline-3-carboxamide
6,7-diethoxy-4-{[2-ethyl-3-({[(1R,2S)-2-hydroxy-2,3-dihydro-1H-inden-1-yl]amino}methyl)phenyl]amino}quinoline-3-carboxamide bis(trifluoroacetate) (salt)
6,7-diethoxy-4-{[2-ethyl-3-({[2-hydroxy-1-(1H-indol-2-ylmethyl)ethyl]amino}methyl)phenyl]amino}quinoline-3-carboxamide bis(trifluoroacetate) (salt)
6,7-diethoxy-4-{[2-ethyl-3-({[(1R)-2-hydroxy-1-phenylethyl]amino}methyl)phenyl]amino}quinoline-3-carboxamide bis(trifluoroacetate) (salt)
6,7-Diethoxy-4-{2-ethyl-3-[(2-hydroxy-1-methylcarbamoyl-propylamino)-methyl]-phenylamino}-quinoline-3-carboxylic acid amide
6,7-diethoxy-4-{[2-ethyl-3-({[(1R,2S)-2-hydroxy-1-(hydroxymethyl)propyl]amino}methyl)phenyl]amino}quinoline-3-carboxamide
6,7-diethoxy-4-{[2-ethyl-3-({[(1R,2R)-2-hydroxy-1-(hydroxymethyl)propyl]amino}methyl)phenyl]amino}quinoline-3-carboxamide methyl N-(3-{[3-(aminocarbonyl)-6,7-diethoxyquinolin-4-yl]amino}-2-ethylbenzyl)serinate bis(trifluoroacetate)
6,7-diethoxy-4-{[2-ethyl-3-({[2-hydroxy-1-(hydroxymethyl)ethyl]amino}methyl)phenyl]amino}quinoline-3-carboxamide
6,7-diethoxy-4-{[2-ethyl-3-({[1-(hydroxymethyl)-3-methylbutyl]amino}methyl)phenyl]amino}quinoline-3-carboxamide
6,7-diethoxy-4-[(2-ethyl-3-{[(2-pyrrolidin-1-ylethyl)amino]methyl}phenyl)amino]quinoline-3-carboxamide
6,7-diethoxy-4-{[2-ethyl-3-({[(1S,2R)-2-hydroxy-1-(hydroxymethyl)propyl]amino}methyl)phenyl]amino}quinoline-3-carboxamide
6,7-diethoxy-4-{[2-ethyl-3-({[(1S)-1-(hydroxymethyl)-3-methylbutyl]amino}methyl)phenyl]amino}quinoline-3-carboxamide
6,7-diethoxy-4-{[2-ethyl-3-({[1-(hydroxymethyl)butyl]amino}methyl)phenyl]amino}quinoline-3-carboxamide
4-{3-[(1-Carbamoyl-2-hydroxy-propylamino)-methyl]-2-ethyl-phenylamino}-6,7-diethoxy-quinoline-3-carboxylic acid amide
6,7-diethoxy-4-[(2-ethyl-3-{[[(1R,2R)-2-hydroxy-1-methyl-2-phenylethyl](methyl)amino]methyl}phenyl)amino]quinoline-3-carboxamide
6,7-diethoxy-4-[(2-ethyl-3-{[(2-hydroxy-1-methyl-2-phenylethyl)amino]methyl}phenyl)amino]quinoline-3-carboxamide
4-{[3-({[2-(3,4-dihydroxyphenyl)-2-hydroxyethyl]amino}methyl)-2-ethylphenyl]amino}-6,7-diethoxyquinoline-3-carboxamide
6,7-diethoxy-4-[(2-ethyl-3-{[(2-hydroxypropyl)amino]methyl}phenyl)amino]quinoline-3-carboxamide
6,7-diethoxy-4-[(2-ethyl-3-{[(2-hydroxy-1-methylethyl)amino]methyl}phenyl)amino]quinoline-3-carboxamide
6,7-diethoxy-4-[(2-ethyl-3-{[(2-hydroxyethyl)amino]methyl}phenyl)amino]quinoline-3-carboxamide
4-[(3-{[(2,3-dihydroxypropyl)amino]methyl}-2-ethylphenyl)amino]-6,7-diethoxyquinoline-3-carboxamide
6,7-diethoxy-4-{[2-ethyl-3-({[2-(hydroxymethyl)phenyl]amino}methyl)phenyl]amino}quinoline-3-carboxamide
4-{[3-({[(1S)-1-benzyl-2-hydroxyethyl]amino}methyl)-2-ethylphenyl]amino}-6,7-diethoxyquinoline-3-carboxamide bis(trifluoroacetate) (salt)
4-{[3-({[2-(dimethylamino)ethyl]amino}methyl)-2-ethylphenyl]amino}-6,7-diethoxyquinoline-3-carboxamide tris(trifluoroacetate)
6,7-diethoxy-4-{[2-ethyl-3-({[4-(methylsulfonyl)phenyl]amino}methyl)phenyl]amino}quinoline-3-carboxamide bis(trifluoroacetate)
6,7-diethoxy-4-{[2-ethyl-3-({[(1S)-2-hydroxy-1-phenylethyl]amino}methyl)phenyl]amino}quinoline-3-carboxamide bis(trifluoroacetate) (salt)
6,7-diethoxy-4-[(2-ethyl-3-{[(2R)-2-(hydroxymethyl)pyrrolidin-1-yl]methyl}phenyl)amino]quinoline-3-carboxamide bis(trifluoroacetate) (salt)
6,7-diethoxy-4-{[2-ethyl-3-({[(1S,2S)-2-hydroxy-1-(hydroxymethyl)-2-phenylethyl]amino}methyl)phenyl]amino}quinoline-3-carboxamide bis(trifluoroacetate) (salt)
6,7-diethoxy-4-[(2-ethyl-3-{[(2-morpholin-4-ylethyl)amino]methyl}phenyl)amino]quinoline-3-carboxamide tris(trifluoroacetate)
6,7-diethoxy-4-{[2-ethyl-3-({[(1R,2S)-2-hydroxy-2-(4-hydroxyphenyl)-1-methylethyl]amino}methyl)phenyl]amino}quinoline-3-carboxamide bis(trifluoroacetate) (salt)
6,7-diethoxy-4-{[2-ethyl-3-({[(1R,2R)-2-hydroxy-1-(hydroxymethyl)-2-phenylethyl]amino}methyl)phenyl]amino}quinoline-3-carboxamide bis(trifluoroacetate) (salt)
6,7-Diethoxy-4-{2-ethyl-3-[(2-hydroxy-1-hydroxymethyl-2-phenyl-ethylamino)-methyl]-phenylamino}-quinoline-3-carboxylic acid amide bis(trifluoroacetate)
4-[(3-{[(2-cyanoethyl)amino]methyl}-2-ethylphenyl)amino]-6,7-diethoxyquinoline-3-carboxamide bis(trifluoroacetate)
6,7-diethoxy-4-{[2-ethyl-3-({[1-(hydroxymethyl)-2-methylpropyl]amino}methyl)phenyl]amino}quinoline-3-carboxamide bis(trifluoroacetate) (salt)
6,7-diethoxy-4-{[2-ethyl-3-({[4-(methylsulfonyl)benzyl]amino}methyl)phenyl]amino}quinoline-3-carboxamide bis(trifluoroacetate)
tert-butyl (3-{[3-(aminocarbonyl)-6,7-diethoxyquinolin-4-yl]amino}-2-ethylbenzyl)carbamate
4-{[3-(aminomethyl)-2-ethylphenyl]amino}-6,7-diethoxyquinoline-3-carboxamide
4-{[3-(aminomethyl)-2-methylphenyl]amino}-6,7-diethoxyquinoline-3-carboxamide
6,7-diethoxy-4-({2-ethyl-3-[(L-tyrosylamino)methyl]phenyl}amino)quinoline-3-carboxamide bis(trifluoroacetate)
6,7-diethoxy-4-{[3-({[(ethylamino)carbonyl]amino}methyl)-2-methylphenyl]amino}quinoline-3-carboxamide
4-({3-[(acetylamino)methyl]-2-methylphenyl}amino)-6,7-diethoxyquinoline-3-carboxamide
6,7-diethoxy-4-({2-methyl-3-[({[(4-methyl-2,5-dioxoimidazolidin-4-yl)methyl]sulfonyl}amino)methyl]phenyl}amino)quinoline-3-carboxamide
4-({3-[(acetylamino)methyl]-2-ethylphenyl}amino)-6,7-dimethoxyquinoline-3-carboxamide
4-{[2-ethyl-3-({[(ethylamino)carbonyl]amino}methyl)phenyl]amino}-6,7-dimethoxyquinoline-3-carboxamide
4-[(2-ethyl-3-{[(methylsulfonyl)amino]methyl}phenyl)amino]-6,7-dimethoxyquinoline-3-carboxamide
4-({2-ethyl-3-[(L-valylamino)methyl]phenyl}amino)-6,7-dimethoxyquinoline-3-carboxamide
4-[(3-{[(3-cyclohexyl-L-alanyl)amino]methyl}-2-ethylphenyl)amino]-6,7-dimethoxyquinoline-3-carboxamide
6,7-diethoxy-4-({2-ethyl-3-[(L-methionylamino)methyl]phenyl}amino)quinoline-3-carboxamide bis(trifluoroacetate)
6,7-diethoxy-4-({2-ethyl-3-[(L-prolylamino)methyl]phenyl}amino)quinoline-3-carboxamide bis(trifluoroacetate)
6,7-diethoxy-4-({2-ethyl-3-[(L-threonylamino)methyl]phenyl}amino)quinoline-3-carboxamide bis(trifluoroacetate)
N˜1˜-(3-{[3-(aminocarbonyl)-6,7-diethoxyquinolin-4-yl]amino}-2-ethylbenzyl)-L-alpha-glutamine bis(trifluoroacetate)
6,7-diethoxy-4-({2-ethyl-3-[(L-valylamino)methyl]phenyl}amino)quinoline-3-carboxamide bis(trifluoroacetate)
4-({3-[(L-arginylamino)methyl]-2-ethylphenyl}amino)-6,7-diethoxyquinoline-3-carboxamide tris(trifluoroacetate)
4-({3-[(L-alanylamino)methyl]-2-ethylphenyl}amino)-6,7-diethoxyquinoline-3-carboxamide bis(trifluoroacetate)
6,7-diethoxy-4-({2-ethyl-3-[(D-serylamino)methyl]phenyl}amino)quinoline-3-carboxamide bis(trifluoroacetate)
4-[(3-{[(3-cyclohexyl-L-alanyl)amino]methyl}-2-ethylphenyl)amino]-6,7-diethoxyquinoline-3-carboxamide bis(trifluoroacetate)
6,7-diethoxy-4-{[2-ethyl-3-({[(4S)-1,3-thiazolidin-4-ylcarbonyl]amino}methyl)phenyl]amino}quinoline-3-carboxamide bis(trifluoroacetate)
6,7-diethoxy-4-{[2-ethyl-3-({[(4R)-4-hydroxy-L-prolyl]amino}methyl)phenyl]amino}quinoline-3-carboxamide bis(trifluoroacetate) (salt)
6,7-diethoxy-4-({2-ethyl-3-[(D-leucylamino)methyl]phenyl}amino)quinoline-3-carboxamide bis(trifluoroacetate)
N˜1˜-(3-{[3-(aminocarbonyl)-6,7-diethoxyquinolin-4-yl]amino}-2-ethylbenzyl)-L-aspartamide bis(trifluoroacetate)
6,7-diethoxy-4-{[2-ethyl-3-({[(2S)-piperidin-2-ylcarbonyl]amino}methyl)phenyl]amino}quinoline-3-carboxamide bis(trifluoroacetate)
4-[(3-{[(3-cyclohexyl-D-alanyl)amino]methyl}-2-ethylphenyl)amino]-6,7-diethoxyquinoline-3-carboxamide bis(trifluoroacetate)
6,7-diethoxy-4-{[2-ethyl-3-({[(2R)-piperidin-2-ylcarbonyl]amino}methyl)phenyl]amino}quinoline-3-carboxamide bis(trifluoroacetate)
4-{[3-({[(2S)-2-aminopent-4-enoyl]amino}methyl)-2-ethylphenyl]amino}-6,7-diethoxyquinoline-3-carboxamide bis(trifluoroacetate)
4-{[3-({[(2S)-azetidin-2-ylcarbonyl]amino}methyl)-2-ethylphenyl]amino}-6,7-diethoxyquinoline-3-carboxamide bis(trifluoroacetate)
6,7-diethoxy-4-[(2-ethyl-3-{[(5-methyl-L-norleucyl)amino]methyl}phenyl)amino]quinoline-3-carboxamide bis(trifluoroacetate)
6,7-diethoxy-4-{[2-ethyl-3-({[(4R)-1,3-thiazolidin-4-ylcarbonyl]amino}methyl)phenyl]amino}quinoline-3-carboxamide bis(trifluoroacetate)
6,7-diethoxy-4-[(2-ethyl-3-{[(4-nitro-D-phenylalanyl)amino]methyl}phenyl)amino]quinoline-3-carboxamide bis(trifluoroacetate)
4-{[3-({[(1-amino-2,3-dihydro-1H-inden-1-yl)carbonyl]amino}methyl)-2-ethylphenyl]amino}-6,7-diethoxyquinoline-3-carboxamide bis(trifluoroacetate)
4-{[3-({[(1-aminocyclohexyl)carbonyl]amino}methyl)-2-ethylphenyl]amino}-6,7-diethoxyquinoline-3-carboxamide bis(trifluoroacetate)
6,7-diethoxy-4-{[2-ethyl-3-({[(3R)-1,2,3,4-tetrahydroisoquinolin-3-ylcarbonyl]amino}methyl)phenyl]amino}quinoline-3-carboxamide bis(trifluoroacetate)
4-{[3-({[(2R)-2-amino-4-phenylbutanoyl]amino}methyl)-2-ethylphenyl]amino}-6,7-diethoxyquinoline-3-carboxamide bis(trifluoroacetate)
6,7-diethoxy-4-{[2-ethyl-3-({[(3S)-1,2,3,4-tetrahydroisoquinolin-3-ylcarbonyl]amino}methyl)phenyl]amino}quinoline-3-carboxamide bis(trifluoroacetate)
6,7-diethoxy-4-[(2-ethyl-3-{[(4-piperidin-4-yl-L-prolyl)amino]methyl}phenyl)amino]quinoline-3-carboxamide tris(trifluoroacetate)
4-[(3-{[(3-amino-L-alanyl)amino]methyl}-2-ethylphenyl)amino]-6,7-diethoxyquinoline-3-carboxamide tris(trifluoroacetate)
6,7-diethoxy-4-({2-ethyl-3-[(D-phenylalanylamino)methyl]phenyl}amino)quinoline-3-carboxamide bis(trifluoroacetate)
4-{[3-({[(2S)-2-amino-4-phenylbutanoyl]amino}methyl)-2-ethylphenyl]amino}-6,7-diethoxyquinoline-3-carboxamide bis(trifluoroacetate)
6,7-diethoxy-4-{[2-ethyl-3-({[(3S)-piperidin-3-ylcarbonyl]amino}methyl)phenyl]amino}quinoline-3-carboxamide bis(trifluoroacetate)
6,7-diethoxy-4-{[2-ethyl-3-({[(3R)-piperidin-3-ylcarbonyl]amino}methyl)phenyl]amino}quinoline-3-carboxamide bis(trifluoroacetate)
4-{[3-({[(2S)-2-amino-2-phenylacetyl]amino}methyl)-2-ethylphenyl]amino}-6,7-diethoxyquinoline-3-carboxamide bis(trifluoroacetate)
6,7-diethoxy-4-({2-ethyl-3-[(L-leucylamino)methyl]phenyl}amino)quinoline-3-carboxamide bis(trifluoroacetate)
6,7-diethoxy-4-({2-ethyl-3-[(D-prolylamino)methyl]phenyl}amino)quinoline-3-carboxamide bis(trifluoroacetate)
4-{[3-({[(2S)-2,5-dihydro-1H-pyrrol-2-ylcarbonyl]amino}methyl)-2-ethylphenyl]amino}-6,7-diethoxyquinoline-3-carboxamide bis(trifluoroacetate)
6,7-diethoxy-4-({2-ethyl-3-[(glycylamino)methyl]phenyl}amino)quinoline-3-carboxamide bis(trifluoroacetate)
4-{[3-({[2-amino-4-(methylsulfinyl)butanoyl]amino}methyl)-2-ethylphenyl]amino}-6,7-diethoxyquinoline-3-carboxamide bis(trifluoroacetate)
6,7-diethoxy-4-{[2-ethyl-3-({[3-(2-furyl)-L-alanyl]amino}methyl)phenyl]amino}quinoline-3-carboxamide bis(trifluoroacetate)
6,7-diethoxy-4-[(2-ethyl-3-{[(3-pyridin-2-yl-L-alanyl)amino]methyl}phenyl)amino]quinoline-3-carboxamide tris(trifluoroacetate)
6,7-diethoxy-4-{[2-ethyl-3-({[3-(2-thienyl)-L-alanyl]amino}methyl)phenyl]amino}quinoline-3-carboxamide bis(trifluoroacetate)
6,7-diethoxy-4-{[2-ethyl-3-({[3-(1,3-thiazol-4-yl)-L-alanyl]amino}methyl)phenyl]amino}quinoline-3-carboxamide tris(trifluoroacetate)
4-{[3-({[(2S)-2-amino-2-cyclopentylacetyl]amino}methyl)-2-ethylphenyl]amino}-6,7-diethoxyquinoline-3-carboxamide bis(trifluoroacetate)
4-{[3-({[(2S)-2-aminopent-4-ynoyl]amino}methyl)-2-ethylphenyl]amino}-6,7-diethoxyquinoline-3-carboxamide bis(trifluoroacetate)
6,7-diethoxy-4-({2-ethyl-3-[(L-norvalylamino)methyl]phenyl}amino)quinoline-3-carboxamide bis(trifluoroacetate)
4-{[3-({[(2R)-2-amino-2-phenylacetyl]amino}methyl)-2-ethylphenyl]amino}-6,7-diethoxyquinoline-3-carboxamide bis(trifluoroacetate)
6,7-diethoxy-4-{[2-ethyl-3-({[(4R)-4-hydroxy-D-prolyl]amino}methyl)phenyl]amino}quinoline-3-carboxamide bis(trifluoroacetate) (salt)
4-({3-[(beta-alanylamino)methyl]-2-ethylphenyl}amino)-6,7-diethoxyquinoline-3-carboxamide bis(trifluoroacetate)
6,7-diethoxy-4-[(2-ethyl-3-{[(3-pyridin-3-yl-L-alanyl)amino]methyl}phenyl)amino]quinoline-3-carboxamide tris(trifluoroacetate)
6,7-diethoxy-4-[(2-ethyl-3-{[(3-pyridin-3-yl-D-alanyl)amino]methyl}phenyl)amino]quinoline-3-carboxamide tris(trifluoroacetate)
4-{[3-({[N˜5˜-(aminocarbonyl)-L-ornithyl]amino}methyl)-2-ethylphenyl]amino}-6,7-diethoxyquinoline-3-carboxamide bis(trifluoroacetate)
6,7-diethoxy-4-[(2-ethyl-3-{[(5-methyl-D-norleucyl)amino]methyl}phenyl)amino]quinoline-3-carboxamide bis(trifluoroacetate)
4-[(3-{[(2,3-dihydro-1H-isoindol-1-ylcarbonyl)amino]methyl}-2-ethylphenyl)amino]-6,7-diethoxyquinoline-3-carboxamide bis(trifluoroacetate)
6,7-diethoxy-4-({2-ethyl-3-[(L-isoleucylamino)methyl]phenyl}amino)quinoline-3-carboxamide bis(trifluoroacetate)
6,7-diethoxy-4-({2-ethyl-3-[(D-valylamino)methyl]phenyl}amino)quinoline-3-carboxamide bis(trifluoroacetate)
4-{[3-({[(1-aminocyclopentyl)carbonyl]amino}methyl)-2-ethylphenyl]amino}-6,7-diethoxyquinoline-3-carboxamide bis(trifluoroacetate)
4-{[2-ethyl-3-(hydroxymethyl)phenyl]amino}-7-{3-[isobutyryl(isopropyl)amino]propoxy}-6-methoxyquinoline-3-carboxamide
7-{3-[acetyl(isopropyl)amino]propoxy}-4-{[2-ethyl-3-(hydroxymethyl)phenyl]amino}-6-methoxyquinoline-3-carboxamide
6-[2-(acetylamino)ethoxy]-4-[(2-ethylphenyl)amino]-7-methoxyquinoline-3-carboxamide
6-{2-[acetyl(methyl)amino]ethoxy}-4-[(2-ethylphenyl)amino]-7-methoxyquinoline-3-carboxamide
6-{2-[acetyl(isopropyl)amino]ethoxy}-4-[(2-ethylphenyl)amino]-7-methoxyquinoline-3-carboxamide
4-[(2-ethylphenyl)amino]-6-{2-[isobutyryl(methyl)amino]ethoxy}-7-methoxyquinoline-3-carboxamide
4-[(2-ethylphenyl)amino]-6-{2-[isobutyryl(isopropyl)amino]ethoxy}-7-methoxyquinoline-3-carboxamide
7-{3-[acetyl(methyl)amino]propoxy}-4-{[2-ethyl-3-(hydroxymethyl)phenyl]amino}-6-methoxyquinoline-3-carboxamide
4-{[2-ethyl-3-(hydroxymethyl)phenyl]amino}-7-{3-[isobutyryl(methyl)amino]propoxy}-6-methoxyquinoline-3-carboxamide
7-{3-[acetyl(cyclopropyl)amino]propoxy}-4-{[2-ethyl-3-(hydroxymethyl)phenyl]amino}-6-methoxyquinoline-3-carboxamide
7-{3-[cyclopropyl(isobutyryl)amino]propoxy}-4-{[2-ethyl-3-(hydroxymethyl)phenyl]amino}-6-methoxyquinoline-3-carboxamide
7-[3-(acetylamino)propoxy]-4-{[2-ethyl-3-(hydroxymethyl)phenyl]amino}-6-methoxyquinoline-3-carboxamide
4-{[2-ethyl-3-(hydroxymethyl)phenyl]amino}-7-[3-(isobutyrylamino)propoxy]-6-methoxyquinoline-3-carboxamide
6-{2-[(cyclopropylcarbonyl)(methyl)amino]ethoxy}-4-[(2-ethylphenyl)amino]-7-methoxyquinoline-3-carboxamide
6-{2-[(cyclopropylcarbonyl)(isopropyl)amino]ethoxy}-4-[(2-ethylphenyl)amino]-7-methoxyquinoline-3-carboxamide
4-{[2-ethyl-3-(hydroxymethyl)phenyl]amino}-7-{3-[isopropyl(methylsulfonyl)amino]propoxy}-6-methoxyquinoline-3-carboxamide
4-{[2-ethyl-3-(hydroxymethyl)phenyl]amino}-6-methoxy-7-{3-[(methylsulfonyl)amino]propoxy}quinoline-3-carboxamide tert-butyl {3-[(3-(aminocarbonyl)-4-{[2-ethyl-3-(hydroxymethyl)phenyl]amino}-6-methoxyquinolin-7-yl)oxy]propyl}isopropylcarbamate
4-{[2-ethyl-3-(hydroxymethyl)phenyl]amino}-7-(3-{isopropyl[(isopropylamino)carbonyl]amino}propoxy)-6-methoxyquinoline-3-carboxamide
7-[3-(cyclopropylamino)propoxy]-4-{[2-ethyl-3-(hydroxymethyl)phenyl]amino}-6-methoxyquinoline-3-carboxamide
6-[3-(cyclopropylamino)propoxy]-4-{[2-ethyl-3-(hydroxymethyl)phenyl]amino}-7-methoxyquinoline-3-carboxamide
7-{3-[(2-cyanoethyl)(methyl)amino]propoxy}-4-{[3-(hydroxymethyl)-2-methylphenyl]amino}-6-methoxyquinoline-3-carboxamide bis(trifluoroacetate) (salt)
4-{[3-(hydroxymethyl)-2-methylphenyl]amino}-6-methoxy-7-[3-(2-methylpiperidin-1-yl)propoxy]quinoline-3-carboxamide
7-{3-[(2-cyanoethyl)(methyl)amino]propoxy}-4-{[3-(hydroxymethyl)-2-methylphenyl]amino}-6-methoxyquinoline-3-carboxamide
4-{[3-(hydroxymethyl)-2-methylphenyl]amino}-7-[3-(3-hydroxypiperidin-1-yl)propoxy]-6-methoxyquinoline-3-carboxamide
4-{[3-(hydroxymethyl)-2-methylphenyl]amino}-7-[3-(4-hydroxypiperidin-1-yl)propoxy]-6-methoxyquinoline-3-carboxamide
6-methoxy-4-[(2-methylphenyl)amino]-7-[3-(2-methylpiperidin-1-yl)propoxy]quinoline-3-carboxamide
7-[3-(3-hydroxypiperidin-1-yl)propoxy]-6-methoxy-4-[(2-methylphenyl)amino]quinoline-3-carboxamide
7-[3-(4-hydroxypiperidin-1-yl)propoxy]-6-methoxy-4-[(2-methylphenyl)amino]quinoline-3-carboxamide
4-{[3-(hydroxymethyl)-2-methylphenyl]amino}-7-[3-(3-hydroxypyrrolidin-1-yl)propoxy]-6-methoxyquinoline-3-carboxamide
4-{[2-ethyl-3-(hydroxymethyl)phenyl]amino}-6-methoxy-7-[3-(1H-1,2,4-triazol-1-yl)propoxy]quinoline-3-carboxamide bis(trifluoroacetate) (salt)
7-[2-(cyclopropylamino)ethoxy]-4-{[3-(hydroxymethyl)-2-methylphenyl]amino}-6-methoxyquinoline-3-carboxamide bis(trifluoroacetate) (salt)
6-[2-(cyclopropylamino)ethoxy]-4-{[3-(hydroxymethyl)-2-methylphenyl]amino}-7-methoxyquinoline-3-carboxamide bis(trifluoroacetate) (salt)
6-[2-(cyclopropylamino)ethoxy]-4-[(4-ethylphenyl)amino]-7-methoxyquinoline-3-carboxamide
6-[2-(cyclopropylamino)ethoxy]-4-[(3-ethylphenyl)amino]-7-methoxyquinoline-3-carboxamide
6-[2-(cyclopropylamino)ethoxy]-7-methoxy-4-[(2-methylphenyl)amino]quinoline-3-carboxamide bis(trifluoroacetate)
6-{2-[(2-cyanoethyl)amino]ethoxy}-4-{[3-(hydroxymethyl)-2-methylphenyl]amino}-7-methoxyquinoline-3-carboxamide bis(trifluoroacetate) (salt)
6-[3-(cyclopropylamino)propoxy]-4-[(2-ethylphenyl)amino]-7-methoxyquinoline-3-carboxamide bis(trifluoroacetate)
6-{3-[(cyanomethyl)amino]propoxy}-4-[(2-ethylphenyl)amino]-7-methoxyquinoline-3-carboxamide
6-[3-(Carbamoylmethyl-amino)-propoxy]-4-(2-ethyl-phenylamino)-7-methoxy-quinoline-3-carboxylic acid amide bis(trifluoroacetate)
methyl N-[3-({3-(aminocarbonyl)-4-[(2-ethylphenyl)amino]-7-methoxyquinolin-6-yl}oxy)propyl]glycinate bis(trifluoroacetate)
7-(3-cyanopropoxy)-4-{[2-ethyl-3-(hydroxymethyl)phenyl]amino}-6-methoxyquinoline-3-carboxamide trifluoroacetate (salt)
2-[(3-(aminocarbonyl)-4-{[2-ethyl-3-(hydroxymethyl)phenyl]amino}-6-methoxyquinolin-7-yl)oxy]ethyl acetate trifluoroacetate (salt)
6-[2-(cyclopropylamino)ethoxy]-4-[(2-ethylphenyl)amino]-7-methoxyquinoline-3-carboxamide
7-[3-(2,5-dioxopyrrolidin-1-yl)propoxy]-4-{[2-ethyl-3-(hydroxymethyl)phenyl]amino}-6-methoxyquinoline-3-carboxamide
4-{[2-ethyl-3-(hydroxymethyl)phenyl]amino}-6-methoxy-7-[3-(3-methyl-2,5-dioxoimidazolidin-1-yl)propoxy]quinoline-3-carboxamide
4-{[2-ethyl-3-(hydroxymethyl)phenyl]amino}-6-methoxy-7-[3-(3,4,4-trimethyl-2,5-dioxoimidazolidin-1-yl)propoxy]quinoline-3-carboxamide
7-(cyclopentyloxy)-4-{[2-ethyl-3-(hydroxymethyl)phenyl]amino}-6-methoxyquinoline-3-carboxamide
6-(cyclopentyloxy)-4-[(2-ethylphenyl)amino]-7-methoxyquinoline-3-carboxamide
1-{3-[(3-(aminocarbonyl)-4-{[3-(hydroxymethyl)-2-methylphenyl]amino}-6-methoxyquinolin-7-yl)oxy]propyl}-1-methylpyrrolidinium iodide
tert-butyl 4-[(3-(aminocarbonyl)-4-{[2-ethyl-3-(hydroxymethyl)phenyl]amino}-6-methoxyquinolin-7-yl)oxy]piperidine-1-carboxylate
tert-butyl 4-({3-(aminocarbonyl)-4-[(2-ethylphenyl)amino]-7-methoxyquinolin-6-yl}oxy)piperidine-1-carboxylate
3-(aminocarbonyl)-4-[(2-ethylphenyl)amino]-7-methoxyquinolin-6-yl propane-2-sulfonate
4-{[2-ethyl-3-(hydroxymethyl)phenyl]amino}-6-methoxy-7-(piperidin-4-yloxy)quinoline-3-carboxamide
4-[(2-ethylphenyl)amino]-7-methoxy-6-(piperidin-4-yloxy)quinoline-3-carboxamide
6-[3-(cyclopropylamino)-2-hydroxypropoxy]-4-[(2-ethylphenyl)amino]-7-methoxyquinoline-3-carboxamide
6-{3-[(2-cyanoethyl)amino]-2-hydroxypropoxy}-4-[(2-ethylphenyl)amino]-7-methoxyquinoline-3-carboxamide
4-[(2-ethylphenyl)amino]-6-[2-hydroxy-3-(2-hydroxypyrrolidin-1-yl)propoxy]-7-methoxyquinoline-3-carboxamide
4-[(2-ethylphenyl)amino]-6-(2-hydroxy-3-piperazin-1-ylpropoxy)-7-methoxyquinoline-3-carboxamide
6-{[(2R)-3-(cyclopropylamino)-2-hydroxy-2-methylpropyl]oxy}-4-[(2-ethylphenyl)amino]-7-methoxyquinoline-3-carboxamide
6-{[(2S)-3-(cyclopropylamino)-2-hydroxy-2-methylpropyl]oxy}-4-[(2-ethylphenyl)amino]-7-methoxyquinoline-3-carboxamide
6-[3-(cyclopropylamino)-2-hydroxypropoxy]-4-{[2-ethyl-3-(hydroxymethyl)phenyl]amino}-7-methoxyquinoline-3-carboxamide
6-{[(2R)-3-(cyclopropylamino)-2-hydroxypropyl]oxy}-4-[(2-ethylphenyl)amino]-7-methoxyquinoline-3-carboxamide
6-{[(2S)-3-(cyclopropylamino)-2-hydroxypropyl]oxy}-4-[(2-ethylphenyl)amino]-7-methoxyquinoline-3-carboxamide
3-(aminocarbonyl)-4-[(2-ethylphenyl)amino]-7-methoxyquinolin-6-yl 2-methylpropanoate
6,7-diethoxy-4-[(4-methyl-1-oxo-1,2-dihydroisoquinolin-5-yl)amino]quinoline-3-carboxamide
6,7-diethoxy-4-[(4-methyl-1-oxo-1,2,3,4-tetrahydroisoquinolin-5-yl)amino]quinoline-3-carboxamide
tert-butyl 5-{[3-(aminocarbonyl)-6,7-diethoxyquinolin-4-yl]amino}-3,4-dihydroisoquinoline-2(1H)-carboxylate
6,7-diethoxy-4-(1,2,3,4-tetrahydroisoquinolin-5-ylamino)quinoline-3-carboxamide
4-{[3-(azidomethyl)-2-ethylphenyl]amino}-6-[3-(cyclopropylamino)propoxy]-7-methoxyquinoline-3-carboxamide
4-{[3-(aminomethyl)-2-ethylphenyl]amino}-6-[3-(cyclopropylamino)propoxy]-7-methoxyquinoline-3-carboxamide
4-{[3-(aminomethyl)-2-ethylphenyl]amino}-7-{3-[isobutyryl(isopropyl)amino]propoxy}-6-methoxyquinoline-3-carboxamide
4-{[3-(azidomethyl)-2-ethylphenyl]amino}-6-[3-(cyclopropylamino)-2-hydroxypropoxy]-7-methoxyquinoline-3-carboxamide
4-{[3-(aminomethyl)-2-ethylphenyl]amino}-6-[3-(cyclopropylamino)-2-hydroxypropoxy]-7-methoxyquinoline-3-carboxamide
4-({3-[(acetylamino)methyl]-2-ethylphenyl}amino)-6-{3-[acetyl(cyclopropyl)amino]-2-hydroxypropoxy}-7-methoxyquinoline-3-carboxamide
6-[3-(cyclopropylamino)-2-hydroxypropoxy]-4-{[2-ethyl-3-(1H-imidazol-1-ylmethyl)phenyl]amino}-7-methoxyquinoline-3-carboxamide
6-[3-(cyclopropylamino)-2-hydroxypropoxy]-4-{[2-ethyl-3-(1H-pyrazol-1-ylmethyl)phenyl]amino}-7-methoxyquinoline-3-carboxamide
6-{[(2S)-3-(cyclopropylamino)-2-hydroxypropyl]oxy}-4-{[2-ethyl-3-(morpholin-4-ylmethyl)phenyl]amino}-7-methoxyquinoline-3-carboxamide
amino {6,7-diethoxy-4-[(2-ethylphenyl)amino]quinolin-3-yl}methanol
6-[3-(cyclopropylamino)propoxy]-4-{[2-ethyl-3-(1H-imidazol-1-ylmethyl)phenyl]amino}-7-methoxyquinoline-3-carboxamide
4-{[2-ethyl-3-(1H-imidazol-1-ylmethyl)phenyl]amino}-6-methoxy-7-(2-methoxyethoxy)quinoline-3-carboxamide
6-(ethylamino)-4-{[2-ethyl-3-(1H-imidazol-1-ylmethyl)phenyl]amino}-7-methoxyquinoline-3-carboxamide
6-[(2,2-dimethoxyethyl)amino]-4-[(2-ethylphenyl)amino]-7-methoxyquinoline-3-carboxamide
6-[(3,3-diethoxypropyl)amino]-4-[(2-ethylphenyl)amino]-7-methoxyquinoline-3-carboxamide
tert-butyl [2-({3-(aminocarbonyl)-4-[(2-ethylphenyl)amino]-7-methoxyquinolin-6-yl}amino)ethyl]carbamate
tert-butyl {2-[(3-(aminocarbonyl)-4-{[2-ethyl-3-(hydroxymethyl)phenyl]amino}-7-methoxyquinolin-6-yl)amino]ethyl}carbamate
6-{[3-(cyclopropylamino)propyl]amino}-4-[(2-ethylphenyl)amino]-7-methoxyquinoline-3-carboxamide
4-(2,3-dihydro-1H-inden-1-ylamino)-6,7-dimethoxyquinoline-3-carboxamide
6,7-diethoxy-4-[(2-methylcyclohexyl)amino]quinoline-3-carboxamide
4-{[(3S)-1-(cyanoacetyl)pyrrolidin-3-yl]amino}-6,7-dimethoxyquinoline-3-carboxamide
4-{[(3S)-1-(cyanoacetyl)piperidin-3-yl]amino}-6,7-dimethoxyquinoline-3-carboxamide and pharmaceutically acceptable salts and solvates of any one thereof.

Where the compounds according to the invention contain one or more asymmetrically substituted carbon atoms, the invention includes all stereoisomers, including enantiomers and diastereomers, and mixtures including racemic mixtures thereof. Tautomers and mixtures thereof are also included.

Racemates may be separated into individual enantiomers using known procedures (cf. Advanced Organic Chemistry: 3rd Edition: author J March, p 104-107). A suitable procedure involves formation of diastereomeric derivatives by reaction of the racemic material with a chiral auxiliary, followed by separation, for example by chromatography, of the diastereomers and then cleavage of the auxiliary species.

The compounds according to the invention may be provided as pharmaceutically acceptable salts. Suitable pharmaceutically acceptable salts include base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. In another aspect, where the compound is sufficiently basic, suitable salts include acid addition salts such as methanesulphonate, fumarate, hydrochloride, hydrobromide, citrate, maleate and salts formed with phosphoric and sulphuric acid.

The present invention further provides a process for the preparation of a compound of formula (I) as defined above, or a pharmaceutically acceptable salt thereof, which comprises:

(a) reaction of a compound of formula (II):

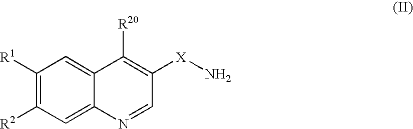

in which R¹ and R² are as defined in formula (I) or are protected derivatives thereof and R²⁰ is a leaving group, with a compound of formula (III):

R^{x}—N(R^{a})H (III)

in which R^{x} and R^{a} are as defined in formula (I) or a protected derivative thereof, or (b) for compounds of formula (I) where R¹ and/or R² are groups Y(CR³₂)ₚNR⁴R⁵, Y(CR₁₂)ₚCONR⁴R⁵, Y(CR³)ₚCO₂R⁶, Y(CR³₂)ₚOR⁶ or Y(CR³₂)ₚR⁶ where Y is oxygen, reaction of a compound of formula (IV):

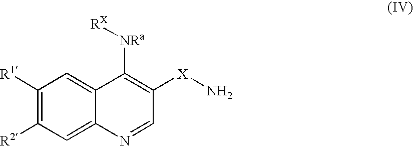

where the R^{1′} or R^{2′} to be converted into a group Y(CR³₂)ₚNR⁴R⁵, Y(CR³₂)ₚCONR⁴R⁵, Y(CR³)ₚCO₂R⁶, Y(CR³₂)ₚOR⁶ or Y(CR³₂)ₚR⁶ is hydroxy and the other R^{1′} or R^{2′} together with R^{x} are as defined above for process (a) with a compound of formula (V):

L-(CR³₂)ₚR²¹ (V)

where R²¹ is NR⁴R⁵, CONR⁴R⁵, CO₂R⁶, OR⁶ or R⁶ and R⁴, R⁵ and R⁶ are as defined in formula (I) or are protected derivatives thereof,
and optionally thereafter process (a) or (b)
removing any protecting groups
converting a compound of formula (I) into a further compound of formula (I)
forming a pharmaceutically acceptable salt or solvate.

In process (a) the group R²⁰ is a leaving group such as halogen, in particular chloro. The reaction can be carried out in an inert solvent such as DMF at elevated temperature, for example at about 100° C.

In process (b) the leaving group L is preferably halogen, in particular chloro. The reaction can be carried out in the presence of a base such as cesium carbonate in an inert solvent such as DMF or ethanol.

Compounds of formula (II) can be prepared by reacting compounds of formula (VI):

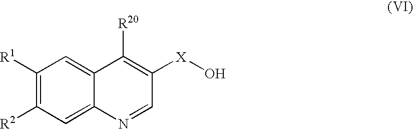

in which R¹, R² and R²⁰ are as defined in formula (II) with a chlorinating agent such as thionyl chloride, and reaction of the corresponding acid chloride with ammonia.

Compounds of formula (VI) can be prepared using methods conventional in the art.

Compounds of formula (I) can be converted into further compounds of formula (I) using standard procedures conventional in the art.

Examples of the types of conversion reactions that may be used include introduction of a substituent by means of an aromatic substitution reaction, reduction of substituents, alkylation of substituents and oxidation of substituents. The reagents and reaction conditions for such procedures are well known in the chemical art and are illustrated in the Examples below. By way of example, a hydroxy group may be replaced with a chloro group by reaction with a chlorinating agent such as thionyl chloride and the chloro group may itself undergo nucleophilic substitution. Alternatively a chloro substituent may be treated with sodium azide to replace the chloro group with an azido group which in turn may be reduced to an amine group. Amine groups may conveniently be acylated with acid chlorides or isocyanates and converted into amides by treatment with appropriate acids.

It will be appreciated that certain functional groups may need to be protected using standard protecting groups. The protection and deprotection of functional groups is for example, described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

Diseases mediated by JAK3 include inflammatory, immunological, and bronchopulmonary disorders.

The present invention also relates to a pharmaceutical composition for (a) treating or preventing a disorder or condition selected from organ transplant rejection, lupus, multiple sclerosis, rheumatoid arthritis, psoriasis, Type I diabetes and complications from diabetes, cancer, asthma, rhinitis, atopic dermatitis, autoimmune thyroid disorders, ulcerative colitis, Crohn's disease, Alzheimer's disease, leukemia, and other autoimmune diseases or (b) the inhibition of protein tyrosine kinases or Janus kinase 3 (JAK3) in a mammal, including a human, comprising an amount of a compound of formula I or a pharmaceutically acceptable salt thereof, effective in such disorders or conditions and a pharmaceutically acceptable carrier.

Preferably the compounds of the invention are used for the treatment of asthma, rheumatoid arthritis, and host versus graft rejection/transplantation.

The present invention also relates to a pharmaceutical composition for (a) treating or preventing a disorder or condition selected from organ transplant rejection, lupus, multiple sclerosis, rheumatoid arthritis, psoriasis, Type I diabetes and complications from diabetes, cane, asthma, rhinitis, atopic dermatitis, autoimmune thyroid disorders, ulcerative colitis, Crohn's disease, Alzheimer's disease, leukemia, and other autoimmune diseases or (b) the inhibition of protein tyrosine kinases or Janus kinase 3 (JAK3) in a mammal, including a human, comprising an amount of a compound of formula I or a pharmaceutically acceptable salt thereof, alone or in combination with a T-cell immunosuppresant or anti-inflammatory agents, effective in such disorders or conditions and a pharmaceutically acceptable carrier.

The present invention also relates to a method for the inhibition of protein tyrosine kinases or Janus Kinase 3 (JAK3) in a mammal, including human, comprising administering to said mammal an effective amount of a compound of formula I or a pharmaceutically acceptable salt thereof.

The dose of the compound to be administered will depend on the relevant indication, the age, weight and sex of the patient and may be determined by a physician. The dosage will preferably be in the range of from 0.1 mg/kg to 100 mg/kg.

The compounds may be administered topically, e.g. to the lung and/or the airways, in the form of solutions, suspensions, HFA aerosols or dry powder formulations, e.g. formulations in the inhaler device known as the Turbuhaler®; or systemically, e.g. by oral administration in the form of tablets, pills, capsules, syrups, powders or granules, or by parenteral administration, e.g. in the form of sterile parenteral solutions or suspensions, or by rectal administration, e.g. in the form of suppositories.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurized HFA aerosols of the compounds of the invention may be administered by oral or nasal inhalation. For inhalation the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (e.g. oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol, or an other polyol. Suitable carriers are sugars, e.g. lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound, with or without a carrier substance, is delivered to the patient.

For oral administration the active compound may be admixed with an adjuvant or a carrier, e.g. lactose, saccharose, sorbitol, mannitol; a starch, e.g. potato starch, corn starch or amylopectin; a cellulose derivative; a binder, e.g. gelatine or polyvinylpyrrolidone, and/or a lubricant, e.g. magnesium stearate, calcium stearate, polyethylene glycol, a wax, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved in a readily volatile organic solvent.

For the preparation of soft gelatine capsules, the compound may be admixed with e.g. a vegetable oil or polyethylene glycol. Hard gelatine capsules may contain granules of the compound using either the above mentioned excipients for tablets. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The term ‘medical therapy’ as used herein is intended to include prophylactic, diagnostic and therapeutic regimens carried out in vivo or ex vivo on humans or other mammals. The terms “therapeutic” and “therapeutically” will be understood accordingly.

The following Examples illustrate the invention.

### General Methods

All reactions were performed in dried glassware in an argon atmosphere at room temperature, unless otherwise noted. All solvents and reagents and solvents were used as received. Merck Silica gel 60 (0.040-0.063 mm) was used for preparative silica gel chromatography. A Kromasil KR-100-5-C18 column (250×20 mm, Akzo Nobel) and mixtures of acetonitrile/water at a flow rate of 10 ml/min was used for preparative HPLC. Reactions were monitored at 254 nm by analytical HPLC, using a Kromasil C-18 column (150×4.6 mm) and a gradient (containing 0.1% trifluoroacetic acid) of 5 to 100% of acetonitrile in water at a flow rate of 1 ml/min. Evaporations of solvents were performed under reduced pressure using a rotary evaporator at a maximum temperature of 40° C. Products were dried under reduced pressure at 40° C.

¹H-NMR spectra were recorded on a Varian Inova-400 or Unity-500+ instrument. The central solvent peak of chloroform-d (δ_{H} 7.27 ppm), dimethylsulfoxide-d₆ (δ_{H} 2.50 ppm) or methanol-d₄ (δ_{H} 3.35 ppm) were used as internal references. Low resolution mass spectra obtained on a Hewlett Packard 1100 LC-MS system equipped with a APCI ionisation chamber.

Merck Silica gel 60 (0.040-0.063 mm) was used for preparative silica gel chromatography. A Kromasil KR-100-5-C18 column (250×20 mm, Akzo Nobel) and mixtures of acetonitrile/water at a flow rate of 10 ml/min was used for preparative HPLC. Reactions were monitored at 254 nm by analytical HPLC, using a Kromasil C-18 column (150×4.6 mm) and a gradient (containing 0.1% trifluoroacetic acid) of 5 to 100% of acetonitrile in water at a flow rate of 1 ml/min. Evaporations of solvents were performed under reduced pressure using a rotary evaporator at a maximum temperature of 40° C. Products were dried under reduced pressure at 40° C.

### EXAMPLE 1

### 6,7-diethoxy-4-{[2-ethyl-3-(1H-imidazol-1-ylmethyl)phenyl]amino}quinoline-3-carboxamide

### a) 6,7-diethoxy-4-{[2-ethyl-3-(hydroxymethyl)phenyl]amino}quinoline-3-carboxamide

The title compound was prepared according to the method described in WO 02/092571

### b) 4-{[3-(chloromethyl)-2-ethylphenyl]amino}-6,7-diethoxyquinoline-3-carboxamide

To a suspension of 6,7-diethoxy-4-{[2-ethyl-3-(hydroxymethyl)phenyl]amino}quinoline-3-carboxamide (1.1 g, 2.7 mmol) in CH₂Cl₂ (7 ml) was added thionyl chloride (0.7 g, 5.77 mmol). After fifteen minutes the suspension was dissolved. Azeotropic evaporation of excess thionyl chloride with toluene gave the title compound 1.15 g (100%) as a yellow powder.

¹H NMR (400 MHz, CDCl₃): δ 12.5 (1H, s); 9.12 (1H, s); 8.69 (1H, br s); 8.08 (1H, br s); 7.52 (1H, d); 7.45 (1H, s); 7.33 (1H, t); 7.23 (1H, d); 6.63 (1H, s); 4.92 (2H, s); 4.18 (2H, q); 3.72 (2H, br s); 2.83 (2H, br s); 1.39 (3H, t); 1.17 (3H, t); 1.05 (3H, t);

APCI-LC/MS m/z: 428.4 [MH⁺]

### 6,7-diethoxy-4-{[2-ethyl-3-(1H-imidazol-1-ylmethyl)phenyl]amino}quinoline-3-carboxamide

Imidazole (110 mg, 1.6 mmol) was added to a solution of 4-{[3-(chloromethyl)-2-ethylphenyl]amino}-6,7-diethoxyquinoline-3-carboxamide (40.2 mg, 0.094 mmol) In 1-methyl-2-pyrrolidinone (2 ml) The mixture was heated for two hours at 70 C, cooled to room temperature and diluted with water. The product was purified by preparative HPLC. After freeze-drying the title compound was obtained as a white powder.

¹H NMR (400 MHz, DMSO-d₆) δ 10.96 (1H, s), 8.88 (1H, s), 8.30 (1H, s), 7.70 (1H, s), 7.63 (1H, s), 7.22 (1H, s), 7.09 (1H, s), 7.04 (2H, t), 6.93 (1H, s), 6.87 (1H, d), 6.60 (2H, d), 6.55 (1H, s), 5.31 (2H, s), 4.14 (2H, q), 2.85 (2H, q), 1.36 (3H, t), 1.02 (5H, dd)

APCI-LC/MS m/z: 460.2 [MH⁺]

The title compounds of examples 2-115 were prepared in analogous manner to example 1.

### EXAMPLE 2

### 6,7-diethoxy-4-{[2-methyl-3-(1H-1,2,4-triazol-1-ylmethyl)phenyl]amino}quinoline-3-carboxamide

APCI LC-MS m/z: 447.5-[MH+]

### EXAMPLE 3

### 6,7-diethoxy-4-{[2-ethyl-3-(morpholin-4-ylmethyl)phenyl]amino}quinoline-3-carboxamide

¹H NMR (400 MHz, DMSO-d₆) δ 10.97 (1H, s), 8.87 (1H, s), 8.29 (1H, s), 7.60 (1H, s), 7.21 (1H, s), 7.03 (2H, d), 6.98 (2H, t), 6.61 (1H, s), 6.56 (2H, d), 4.14 (2H, q), 3.57 (5H, s), 3.51 (3H, s), 2.87 (2H, q), 2.38 (4H, s), 1.36 (3H, t), 1.24 (3H, t), 1.01 (3H, t)

APCI LC-MS m/z: 479.4 [MH+]

### EXAMPLE 4

### 6,7-diethoxy-4-{[3-(1H-imidazol-1-ylmethyl)-2-methylphenyl]amino}quinoline-3-carboxamide

APCI LC-MS m/z: 446.5-[MH+]

### EXAMPLE 5

### 4-{[3-(azidomethyl)-2-methylphenyl]amino}-6,7-diethoxyquinoline-3-carboxamide

APCI LC-MS m/z: 421.5 [MH+]

### EXAMPLE 6

### 6,7-diethoxy-4-{[2-methyl-3-(4H-1,2,4-triazol-4-ylmethyl)phenyl]amino}quinoline-3-carboxamide

APCI LC-MS m/z: 447.5 [MH+]

### EXAMPLE 7

### 4-{[3-({[4-(aminosulfonyl)benzyl]amino}methyl)-2-ethylphenyl]amino}-6,7-dimethoxyquinoline-3-carboxamide

APCI LC-MS m/z: 550.4 [MH+]

### EXAMPLE 8

### 4-({2-ethyl-3-[(1H-1,2,4-triazol-5-ylamino)methyl]phenyl}amino)-6,7-dimethoxyquinoline-3-carboxamide

APCI LC-MS m/z: 448.2 [MH+]

### EXAMPLE 9

### 4-{[2-ethyl-3-(1H-imidazol-1-ylmethyl)phenyl]amino}-6,7-dimethoxyquinoline-3-carboxamide

¹H NMR (399.99 MHz, DMSO-d₆) δ 11.01 (s, 1H), 8.90 (s, 1H), 8.32 (s, 1H), 7.72 (s, 1H), 7.64 (s, 1H), 7.25 (s, 1H), 7.09 (s, 1H), 7.06 (d, 1H), 6.93 (s, 1H), 6.87 (d, 1H), 6.64 (d, 1H), 6.56 (s, 1H), 5.33 (s, 2H), 3.88 (s, 3H), 3.17 (s, 3H), 2.86 (q, 2H), 1.03 (t, 3H)

APCI LC-MS m/z: 432.4 [MH+]

### EXAMPLE 10

### 6,7-diethoxy-4-({2-ethyl-3-[(pyrimidin-2-ylamino)methyl]phenyl}amino)quinoline-3-carboxamide

APCI LC-MS m/z: 487.1 [MH+]

### EXAMPLE 11

### 6,7-diethoxy-4-[(2-ethyl-3-{[(2-hydroxycyclohexyl)amino]methyl}phenyl)amino]quinoline-3-carboxamide

APCI LC-MS m/z: 507.5 [MH+]

### EXAMPLE 12

### 6,7-diethoxy-4-[(2-ethyl-3-{[(3-thienylmethyl)amino]methyl}phenyl)amino]quinoline-3-carboxamide

APCI LC-MS m/z: 505.6 [MH+]

### EXAMPLE 13

### 6,7-diethoxy-4-({2-ethyl-3-[(1H-imidazol-2-ylthio)methyl]phenyl}amino)quinoline-3-carboxamide

APCI LC-MS m/z: 492.6 [MH+]

### EXAMPLE 14

### 6,7-diethoxy-4-{[2-ethyl-3-(thiomorpholin-4-ylmethyl)phenyl]amino}quinoline-3-carboxamide

APCI LC-MS m/z: 495.6 [MH+]

### EXAMPLE 15

### 6,7-diethoxy-4-[(2-ethyl-3-{[(3-thienylmethyl)amino]methyl}phenyl)amino]quinoline-3-carboxamide

APCI LC-MS m/z: 505.6 [MH+]

### EXAMPLE 16

### 4-({2-ethyl-3-[(4-nitro-1H-imidazol-1-yl)methyl]phenyl}amino)-6,7-dimethoxyquinoline-3-carboxamide

APCI LC-MS m/z: 477.2 [MH+]

### EXAMPLE 17

### 4-[(2-ethyl-3-{[4-(hydroxymethyl)-1H-imidazol-1-yl]methyl}phenyl)amino]-6,7-dimethoxyquinoline-3-carboxamide trifluoroacetate (salt)

APCI LC-MS m/z: 462.5 [MH+]

### EXAMPLE 18

### 4-({2-ethyl-3-[(2-methyl-1H-imidazol-1-yl)methyl]phenyl}amino)-6,7-dimethoxyquinoline-3-carboxamide

APCI LC-MS m/z: 446.5 [MH+]

### EXAMPLE 19

### 1-(3-{[3-(aminocarbonyl)-6,7-dimethoxyquinolin-4-yl]amino}-2-ethylbenzyl)-1H-imidazole-4-carboxylic acid

APCI LC-MS m/z: 476.5 [MH+]

### EXAMPLE 20

### 4-({3-[(cyclopentylamino)methyl]-2-ethylphenyl}amino)-6,7-dimethoxyquinoline-3-carboxamide

¹NMR (400 MHz, DMSO-d₆) δ 11.03 (1H, s), 8.87 (1H, s), 8.28 (1H, s), 7.59 (1H, s), 7.22 (1H, s), 7.12 (1H, d), 7.00 (1H, t), 6.63 (1H, s), 6.57 (1H, d), 3.87 (3H, s), 3.73 (2H, s), 3.19 (3H, s), 3.03 (1H, t), 2.86 (2H, q), 1.73 (2H, mult), 1.61 (2H, mult), 1.46 (2H, mult), 1.35 (2H, mult), 1.21 (3H, t)

APCI LC-MS m/z: 449.2 [MH+]

### EXAMPLE 21

### 4-{[2-ethyl-3-({[2-(1H-imidazol-4-yl)ethyl]amino}methyl)phenyl]amino}-6,7-dimethoxyquinoline-3-carboxamide bis(trifluoroacetate)

¹H NMR (400 MHz, DMSO-d₆) δ 9.20 (1H, s), 9.00 (2H, d), 8.60 (1H, s), 8.07 (1H, s), 7.52 (2H, s), 7.48 (2H, d), 7.37 (2H, mult), 7.23 (1H, d), 6.67 (1H, s), 4.34 (2H, s), 3.94 (4H, s), 3.40 (3H, t), 3.20 (4H, s), 3.11 (3H, t), 2.81 (2H, s), 1.14 (3H, t)

APCI LC-MS m/z: 475.2 [MH+]

### EXAMPLE 22

### 4-[(2-ethyl-3-{[(2-hydroxy-1,1-dimethylethyl)amino]methyl}phenyl)amino]-6,7-dimethoxyquinoline-3-carboxamide

¹H NMR (400 MHz, DMSO-d₆) δ 11.03 (1H, s), 8.87 (1H, s), 8.28 (1H, s), 7.59 (1H, s), 7.22 (1H, s), 7.12 (1H, d), 7.00 (1H, t), 6.62 (2H, s), 6.58 (2H, d), 4.59 (1H, s), 3.86 (3H, s), 3.68 (2H, s), 3.25 (2H, d), 3.19 (3H, s), 2.87 (2H, d), 1.23 (3H, t), 1.02 (6H, s).

APCI LC-MS m/z: 453.1 [MH+]

### EXAMPLE 23

### 4-({2-ethyl-3-[(1,3-thiazol-2-ylamino)methyl]phenyl}amino)-6,7-dimethoxyquinoline-3-carboxamide

APCI LC-MS m/z: 464.1 [MH+]

### EXAMPLE 24

### 4-[(2-ethyl-3-{[(2-hydroxypropyl)amino]methyl}phenyl)amino]-6,7-dimethoxyquinoline-3-carboxamide

APCI LC-MS m/z: 439.3 [MH+]

### EXAMPLE 25

### 4-[(2-ethyl-3-{[(2-hydroxy-2-phenylethyl)amino]methyl}phenyl)amino]-6,7-dimethoxyquinoline-3-carboxamide bis(trifluoroacetate) (salt)

APCI LC-MS m/z: 501.3 [MH+]

### EXAMPLE 26

### 4-{[2-ethyl-3-({[4-(methylsulfonyl)benzyl]amino}methyl)phenyl]amino}-6,7-dimethoxyquinoline-3-carboxamide

¹H NMR (399.99 MHz, dmso-d₆) δ 11.03 (1H, s), 8.88 (1H, s), 8.34 (1H, s), 7.88 (2H, d), 7.63 (2H, d), 7.57 (1H, s), 7.22 (1H, s), 7.16 (1H, d), 7.03 (1H, t), 6.63 (1H, s), 6.59 (1H, d), 3.87 (3H, s), 3.84 (2H, s), 3.76 (2H, s), 3.18 (6H, s), 2.83 (2H, d), 1.17 (3H, t)

APCI LC-MS m/z: 549.3 [MH+]

### EXAMPLE 27

### 4-({3-[(benzylamino)methyl]-2-ethylphenyl}amino)-6,7-dimethoxyquinoline-3-carboxamide

¹H NMR (399.99 MHz, DMSO-d₆) δ 11.03 (1H, s), 8.88 (1H, br s), 8.27 (1H, br s), 7.60 (1H, s), 7.33 (4H, mult), 7.22 (2H, mult), 7.16 (1H, d), 7.03 (1H, t), 6.63 (1H, s), 6.59 (1H, d), 3.87 (3H, s), 3.73 (4H, d), 3.18 (3H, s), 2.81 (2H, q), 1.16 (3H, t)

APCI LC-MS m/z 471.3 [MH+]

### EXAMPLE 28

### 4-({2-ethyl-3-[(3-methyl-2,5-dioxoimidazolidin-1-yl)methyl]phenyl}amino)-6,7-dimethoxyquinoline-3-carboxamide

APCI LC-MS m/z 478.5 [MH+]

### EXAMPLE 29

### 4-({2-ethyl-3-[(1H-tetrazol-5-ylamino)methyl]phenyl}amino)-6,7-dimethoxyquinoline-3-carboxamide bis(trifluoroacetate)

APCI LC-MS m/z 449.2 [MH+]

### EXAMPLE 30

### 4-({3-[(5-amino-1H-tetrazol-1-yl)methyl]-2-ethylphenyl}amino)-6,7-dimethoxyquinoline-3-carboxamide bis(trifluoroacetate)

APCI LC-MS m/z 449.2 [MH+]

### EXAMPLE 31

### 4-{[2-ethyl-3-({[2-(2-oxoimidazolidin-1-yl)ethyl]amino}methyl)phenyl]amino}-6,7-dimethoxyquinoline-3-carboxamide

¹H NMR (399.99 MHz, DMSO-d₆) δ 11.03 (1H, s), 8.87 (1H, s), 8.28 (1H, br s), 7.60 (1H, br s), 7.22 (1H, s), 7.13 (1H, d), 7.01 (1H, t), 6.62 (1H, s), 6.58 (1H, d), 6.22 (1H, s), 3.87 (3H, s), 3.77 (2H, s), 3.29 (2H, t), 3.19 (5H, mult), 3.14 (2H, t), 2.85 (2H, q).

APCI LC-MS m/z 493.3 [MH+]

### EXAMPLE 32

### 4-{[2-ethyl-3-({[(2S)-2-hydroxycyclohexyl]amino}methyl)phenyl]amino}-6,7-dimethoxyquinoline-3-carboxamide bis(trifluoroacetate) (salt)

APCI LC-MS m/z 479.3 [MH+]

### EXAMPLE 33

### 4-({2-ethyl-3-[(piperidin-4-ylamino)methyl]phenyl}amino)-6,7-dimethoxyquinoline-3-carboxamide tris(trifluoroacetate)

APCI LC-MS m/z 464.3 [MH+]

### EXAMPLE 34

### 4-{[2-ethyl-3-({[(1R)-1-(hydroxymethyl)-3-methylbutyl]amino}methyl)phenyl]amino}-6,7-dimethoxyquinoline-3-carboxamide

APCI LC-MS m/z 481.5 [MH+]

### EXAMPLE 35

### 6,7-diethoxy-4-[(2-ethyl-3-{[4-(3-methoxyphenyl)piperazin-1-yl]methyl}phenyl)amino]quinoline-3-carboxamide

APCI LC-MS m/z: 584.6 [MH+]

### EXAMPLE 36

### 6,7-diethoxy-4-[(2-ethyl-3-{[4-(hydroxymethyl)piperidin-1-yl]methyl}phenyl)amino]quinoline-3-carboxamide

APCI LC-MS m/z: 507.5 [MH+]

### EXAMPLE 37

### 6,7-diethoxy-4-[(2-ethyl-3-{[2-(hydroxymethyl)piperidin-1-yl]methyl}phenyl)amino]quinoline-3-carboxamide

APCI LC-MS m/z: 507.6 [MH+]

### EXAMPLE 38

### 4-{[3-(1,4′-bipiperidin-1′-ylmethyl)-2-ethylphenyl]amino}-6,7-diethoxyquinoline-3-carboxamide

APCI LC-MS m/z: 560.7 [MH+]

### EXAMPLE 39

### 4-[(3-{[4-(aminocarbonyl)piperidin-1-yl]methyl}-2-ethylphenyl)amino]-6,7-diethoxyquinoline-3-carboxamide

APCI LC-MS m/z: 520.5 [MH+]

### EXAMPLE 40

### 4-[(3-{[4-(2-cyanophenyl)piperazin-1-yl]methyl}-2-ethylphenyl)amino]-6,7-diethoxyquinoline-3-carboxamide

APCI LC-MS m/z: 579.7 [MH+]

### EXAMPLE 41

### 4-[(3-{[4-(5-cyanopyridin-2-yl)piperazin-1-yl]methyl}-2-ethylphenyl)amino]-6,7-diethoxyquinoline-3-carboxamide

APCI LC-MS m/z: 580.6 [MH+]

### EXAMPLE 42

### 6,7-diethoxy-4-[(2-ethyl-3-{[(3-furylmethyl)amino]methyl}phenyl)amino]quinoline-3-carboxamide

APCI LC-MS m/z: 489.5 [MH+]

### EXAMPLE 43

### 6,7-diethoxy-4-[(2-ethyl-3-{[4-(2-hydroxyethyl)piperazin-1-yl]methyl}phenyl)amino]quinoline-3-carboxamide

APCI LC-MS m/z: 522.6 [MH+]

### EXAMPLE 44

### 6,7-diethoxy-4-({2-ethyl-3-[(4-hydroxypiperidin-1-yl)methyl]phenyl}amino)quinoline-3-carboxamide

APCI LC-MS m/z: 493.5 [MH+]

### EXAMPLE 45

### 4-{[3-({[2-(1,3-benzodioxol-5-yl)ethyl]amino}methyl)-2-ethylphenyl]amino}-6,7-diethoxyquinoline-3-carboxamide

APCI LC-MS m/z: 557.6 [MH+]

### EXAMPLE 46

### 6,7-diethoxy-4-{[2-ethyl-3-({[2-(2-thienyl)ethyl]amino}methyl)phenyl]amino}quinoline-3-carboxamide

APCI LC-MS m/z: 519.5 [MH+]

### EXAMPLE 47

### 4-{[3-({[(2,5-dimethyl-3-furyl)methyl]amino}methyl)-2-ethylphenyl]amino}-6,7-diethoxyquinoline-3-carboxamide

APCI LC-MS m/z: 517.6 [MH+]

### EXAMPLE 48

### 6,7-diethoxy-4-{[2-ethyl-3-({[3-(2-oxopyrrolidin-1-yl)propyl]amino}methyl)phenyl]amino}quinoline-3-carboxamide

APCI LC-MS m/z: 534.6 [MH+]

### EXAMPLE 49

### 4-{[3-({[2-(3-chlorophenyl)ethyl]amino}methyl)-2-ethylphenyl]amino}-6,7-diethoxyquinoline-3-carboxamide

APCI LC-MS m/z: 547.5 [MH+]

### EXAMPLE 50

### 4-{[3-({[2-(4-chlorophenyl)ethyl]amino}methyl)-2-ethylphenyl]amino}-6,7-diethoxyquinoline-3-carboxamide

APCI LC-MS m/z: 547.6 [MH+]

### EXAMPLE 51

### 4-{[3-({[2-(2-chlorophenyl)ethyl]amino}methyl)-2-ethylphenyl]amino}-6,7-diethoxyquinoline-3-carboxamide

APCI LC-MS m/z: 547.6 [MH+]

### EXAMPLE 52

### 6,7-diethoxy-4-[(2-ethyl-3-{[(2-hydroxy-2-phenylethyl)amino]methyl}phenyl)amino]quinoline-3-carboxamide

APCI LC-MS m/z: 529.6 [MH+]

### EXAMPLE 53

### 4-({3-[(cyclopentylamino)methyl]-2-ethylphenyl}amino)-6,7-diethoxyquinoline-3-carboxamide

APCI LC-MS m/z: 477.5 [MH+]

### EXAMPLE 54

### 6,7-diethoxy-4-{[2-ethyl-3-({[2-(1H-imidazol-4-yl)ethyl]amino}methyl)phenyl]amino}quinoline-3-carboxamide

APCI LC-MS m/z: 503.6 [MH+]

### EXAMPLE 55

### 6,7-diethoxy-4-[(2-ethyl-3-{[4-(2-morpholin-4-ylethyl)piperazin-1-yl]methyl}phenyl)amino]quinoline-3-carboxamide

APCI LC-MS m/z: 591.7 [MH+]

### EXAMPLE 56

### 4-{[3-({[(2,2-dimethyl-1,3-dioxolan-4-yl)methyl]amino}methyl)-2-ethylphenyl]amino}-6,7-diethoxyquinoline-3-carboxamide

APCI LC-MS m/z: 523.5 [MH+]

### EXAMPLE 57

### 6,7-diethoxy-4-({2-ethyl-3-[(1,3-thiazol-2-ylamino)methyl]phenyl}amino)quinoline-3-carboxamide

APCI LC-MS m/z: 492.5 [MH+]

### EXAMPLE 58

### 6,7-diethoxy-4-{[2-ethyl-3-(1,3-thiazolidin-3-ylmethyl)phenyl]amino}quinoline-3-carboxamide

APCI LC-MS m/z: 481.5 [MH+]

### EXAMPLE 59

### 6,7-diethoxy-4-[(2-ethyl-3-{[(2-pyridin-2-ylethyl)amino]methyl}phenyl)amino]quinoline-3-carboxamide

APCI LC-MS m/z: 514.5 [MH+]

### EXAMPLE 60

### 6,7-diethoxy-4-({2-ethyl-3-[(1H-1,2,4-triazol-3-ylamino)methyl]phenyl}amino)quinoline-3-carboxamide

APCI LC-MS m/z: 476.6 [MH+]

### EXAMPLE 61

### 6,7-diethoxy-4-{[2-ethyl-3-({[4-(2-thienyl)benzyl]amino}methyl)phenyl]amino}quinoline-3-carboxamide

APCI LC-MS m/z: 581.5 [MH+]

### EXAMPLE 62

### 4-{[3-({[4-(aminosulfonyl)benzyl]amino}methyl)-2-ethylphenyl]amino}-6,7-diethoxyquinoline-3-carboxamide

APCI LC-MS m/z 578.6 [MH+]

### EXAMPLE 63

### 6,7-diethoxy-4-{[2-ethyl-3-({[2-(1H-indol-3-yl)ethyl]amino}methyl)phenyl]amino}quinoline-3-carboxamide

APCI LC-MS m/z: 552.6 [MH+]

### EXAMPLE 64

### 6,7-diethoxy-4-{[2-ethyl-3-({[3-(4-methylpiperazin-1-yl)propyl]amino}methyl)phenyl]amino}quinoline-3-carboxamide

APCI LC-MS m/z: 549.7 [MH+]

### EXAMPLE 65

### 6,7-diethoxy-4-[(2-ethyl-3-{[(1-ethylpiperidin-3-yl)amino]methyl}phenyl)amino]quinoline-3-carboxamide

APCI LC-MS m/z: 520.6 [MH+]

### EXAMPLE 66

### 6,7-diethoxy-4-[(2-ethyl-3-{[4-(pyridin-4-ylmethyl)piperazin-1-yl]methyl}phenyl)amino]quinoline-3-carboxamide

APCI LC-MS m/z: 569.6 [MH+]

### EXAMPLE 67

### 6,7-diethoxy-4-[(2-ethyl-3-{[(pyridin-4-ylmethyl)amino]methyl}phenyl)amino]quinoline-3-carboxamide

APCI LC-MS m/z: 500.6 [MH+]

### EXAMPLE 68

### 6,7-diethoxy-4-[(2-ethyl-3-{[(pyridin-3-ylmethyl)amino]methyl}phenyl)amino]quinoline-3-carboxamide

APCI LC-MS m/z: 500.6 [MH+]

### EXAMPLE 69

### 4-({3-[(benzylamino)methyl]-2-ethylphenyl}amino)-6,7-diethoxyquinoline-3-carboxamide

APCI LC-MS m/z: 499.5 [MH+]

### EXAMPLE 70

### 6,7-diethoxy-4-[(2-ethyl-3-{[(2-furylmethyl)amino]methyl}phenyl)amino]quinoline-3-carboxamide

APCI LC-MS m/z: 489.6 [MH+]

### EXAMPLE 71

### 6,7-diethoxy-4-[(2-ethyl-3-{[(2-methoxyethyl)amino]methyl}phenyl)amino]quinoline-3-carboxamide

APCI LC-MS m/z: 467.5 [MH+]

### EXAMPLE 72

### 6,7-diethoxy-4-[(2-ethyl-3-{[(2-hydroxypropyl)amino]methyl}phenyl)amino]quinoline-3-carboxamide

APCI LC-MS m/z: 467.5 [MH+]

### EXAMPLE 73

### 6,7-diethoxy-4-{[2-ethyl-3-({[4-(1H-pyrazol-1-yl)benzyl]amino}methyl)phenyl]amino}quinoline-3-carboxamide

APCI LC-MS m/z: 565.6 [MH+]

### EXAMPLE 74

### 4-({3-[({2-[4-(aminosulfonyl)phenyl]ethyl}amino)methyl]-2-ethylphenyl}amino)-6,7-diethoxyquinoline-3-carboxamide

APCI LC-MS m/z: 592.7 [MH+]

### EXAMPLE 75

### 6,7-diethoxy-4-{[2-ethyl-3-({[2-(1-methylpyrrolidin-2-yl)ethyl]amino}methyl)phenyl]amino}quinoline-3-carboxamide

APCI LC-MS m/z: 520.7 [MH+]

### EXAMPLE 76

### 4-[(3-{[(4-chlorobenzyl)amino]methyl}-2-ethylphenyl)amino]-6,7-diethoxyquinoline-3-carboxamide

APCI LC-MS m/z: 533.5 [MH+]

### EXAMPLE 77

### 4-[(3-{[(1-benzylpiperidin-4-yl)amino]methyl}-2-ethylphenyl)amino]-6,7-diethoxyquinoline-3-carboxamide

APCI LC-MS m/z: 582.7 [MH+]

### EXAMPLE 78

### 6,7-diethoxy-4-[(2-ethyl-3-{[(3-methoxybenzyl)amino]methyl}phenyl)amino]quinoline-3-carboxamide

APCI LC-MS m/z: 529.5 [MH+]

### EXAMPLE 79

### 6,7-diethoxy-4-[(2-ethyl-3-{[(4-methoxybenzyl)amino]methyl}phenyl)amino]quinoline-3-carboxamide

APCI LC-MS m/z: 529.7 [MH+]

### EXAMPLE 80

### 6,7-diethoxy-4-{[2-ethyl-3-({[3-(1H-imidazol-1-yl)propyl]amino}methyl)phenyl]amino}quinoline-3-carboxamide

APCI LC-MS m/z: 517.6 [MH+]

### EXAMPLE 81

### 6,7-diethoxy-4-{[2-ethyl-3-({[(1R,2S)-2-hydroxy-2,3-dihydro-1H-inden-1-yl]amino}methyl)phenyl]amino}quinoline-3-carboxamide bis(trifluoroacetate) (salt)

APCI LC-MS m/z 541.5 [MH+]

### EXAMPLE 82

### 6,7-diethoxy-4-{[2-ethyl-3-({[2-hydroxy-1-(1H-indol-2-ylmethyl)ethyl]amino}methyl)phenyl]amino}quinoline-3-carboxamide bis(trifluoroacetate) (salt)

APCI LC-MS m/z 582.5 [MH+]

### EXAMPLE 83

### 6,7-diethoxy-4-{[2-ethyl-3-({[(1R)-2-hydroxy-1-phenylethyl]amino}methyl)phenyl]amino}quinoline-3-carboxamide bis(trifluoroacetate) (salt)

APCI LC-MS m/z 524.5 [MH+]

### EXAMPLE 84

### 6,7-Diethoxy-4-{2-ethyl-3-[(2-hydroxy-1-methylcarbamoyl-propylamino)-m ethyl]-phenylamino}-quinoline-3-carboxylic acid amide bis(trifluoroacetate) (salt)

APCI LC-MS m/z 529.5 [MH+]

### EXAMPLE 85

### 6,7-diethoxy-4-{[2-ethyl-3-({[(1R,2S)-2-hydroxy-1-(hydroxymethyl)propyl]amino}methyl)phenyl]amino}quinoline-3-carboxamide bis(trifluoroacetate) (salt)

APCI LC-MS m/z 497.3 [MH+]

### EXAMPLE 86

### 6,7-diethoxy-4-{[2-ethyl-3-({[(1R,2R)-2-hydroxy-1-(hydroxymethyl)propyl]amino}methyl)phenyl]amino}quinoline-3-carboxamide bis(trifluoroacetate) (salt)

APCI LC-MS m/z 497.5 [MH+]

### EXAMPLE 87

### methyl N-(3-{[3-(aminocarbonyl)-6,7-diethoxyquinolin-4-yl]amino}-2-ethylbenzyl)serinate bis(trifluoroacetate)

APCI LC-MS m/z 511.3 [MH+]

### EXAMPLE 88

### 6,7-diethoxy-4-{[2-ethyl-3-({[2-hydroxy-1-(hydroxymethyl)ethyl]amino}methyl)phenyl]amino}quinoline-3-carboxamide bis(trifluoroacetate) (salt)

APCI LC-MS m/z 483.5 [MH+]

### EXAMPLE 89

### 6,7-diethoxy-4-{[2-ethyl-3-({[1-(hydroxymethyl)-3-methylbutyl]amino}methyl)phenyl]amino}quinoline-3-carboxamide bis(trifluoroacetate) (salt)

APCI LC-MS m/z 509.5 [MH+]

### EXAMPLE 90

### 6,7-diethoxy-4-[(2-ethyl-3-{[(2-pyrrolidin-1-ylethyl)amino]methyl}phenyl)amino]quinoline-3-carboxamide tris(trifluoroacetate)

APCI LC-MS m/z 506.5 [MH+]

### EXAMPLE 91

### 6,7-diethoxy-4-{[2-ethyl-3-({[(1S,2R)-2-hydroxy-1-(hydroxymethyl)propyl]amino}methyl)phenyl]amino}quinoline-3-carboxamide bis(trifluoroacetate) (salt)

APCI LC-MS m/z 497.3 [MH+]

### EXAMPLE 92

### 6,7-diethoxy-4-{[2-ethyl-3-({[(S)-1-(hydroxymethyl)-3-methylbutyl]amino}methyl)phenyl]amino}quinoline-3-carboxamide bis(trifluoroacetate) (salt)

APCI LC-MS m/z 509.5 [MH+]

### EXAMPLE 93

### 6,7-diethoxy-4-{[2-ethyl-3-({[1-(hydroxymethyl)butyl]amino}methyl)phenyl]amino}quinoline-3-carboxamide bis(trifluoroacetate) (salt)

APCI LC-MS m/z 495.5 [MH+]

### EXAMPLE 94

### 4-{3-[(1-Carbamoyl-2-hydroxy-propyl amino)-methyl]-2-ethyl-phenylamino}-6,7-diethoxy-quinoline-3-carboxylic acid amide bis(trifluoroacetate) (salt

APCI LC-MS m/z 510.4 [MH+]

### EXAMPLE 95

### 6,7-diethoxy-4-[(2-ethyl-3-{[[(1R,2R)-2-hydroxy-1-methyl-2-phenylethyl](methyl)amino]methyl}phenyl)amino]quinoline-3-carboxamide bis(trifluoroacetate) (salt)

APCI LC-MS m/z 557.5 [MH+]

### EXAMPLE 96

### 6,7-diethoxy-4-[(2-ethyl-3-{[(2-hydroxy-1-methyl-2-phenylethyl)amino]methyl}phenyl)amino]quinoline-3-carboxamide bis(trifluoroacetate) (salt)

APCI LC-MS m/z 543.5 [MH+]

### EXAMPLE 97

### 4-{[3-({[2-(3,4-dihydroxyphenyl)-2-hydroxyethyl]amino}methyl)-2-ethylphenyl]amino}-6,7-diethoxyquinoline-3-carboxamide bis(trifluoroacetate) (salt)

APCI LC-MS m/z 561.5 [MH+]

### EXAMPLE 98

### 6,7-diethoxy-4-[(2-ethyl-3-{[(2-hydroxypropyl)amino]methyl}phenyl)amino]quinoline-3-carboxamide bis(trifluoroacetate) (salt)

APCI LC-MS m/z 467.5 [MH+]

### EXAMPLE 99

### 6,7-diethoxy-4-[(2-ethyl-3-{[(2-hydroxy-1-methylethyl)amino]methyl}phenyl)amino]quinoline-3-carboxamide bis(trifluoroacetate) (salt)

APCI LC-MS m/z 467.5 [MH+]

### EXAMPLE 100

### 6,7-diethoxy-4-[(2-ethyl-3-{[(2-hydroxyethyl)amino]methyl}phenyl)amino]quinoline-3-carboxamide bis(trifluoroacetate) (salt)

APCI LC-MS m/z 453.5 [MH+]

### EXAMPLE 101

### 4-[(3-{[(2,3-dihydroxypropyl)amino]methyl}-2-ethylphenyl)amino]-6,7-diethoxyquinoline-3-carboxamide bis(trifluoroacetate) (salt)

APCI LC-MS m/z 483.5 [MH+]

### EXAMPLE 102

### 6,7-diethoxy-4-{[2-ethyl-3-({[2-(hydroxymethyl)phenyl]amino}methyl)phenyl]amino}quinoline-3-carboxamide bis(trifluoroacetate) (salt)

APCI LC-MS m/z 515.4 [MH+]

### EXAMPLE 103

### 4-{[3-({[(1S)-1-benzyl-2-hydroxyethyl]amino}methyl)-2-ethylphenyl]amino}-6,7-diethoxyquinoline-3-carboxamide bis(trifluoroacetate) (salt)

APCI LC-MS m/z 549.6 [MH+]

### EXAMPLE 104

### 4-{[3-({[2-(dimethylamino)ethyl]amino}methyl)-2-ethylphenyl]amino}-6,7-diethoxyquinoline-3-carboxamide tris(trifluoroacetate)

APCI LC-MS m/z 543.5 [MH+]

### EXAMPLE 105

### 6,7-diethoxy-4-{[2-ethyl-3-({[4-(methylsulfonyl)phenyl]amino}methyl)phenyl]amino}quinoline-3-carboxamide bis(trifluoroacetate)

APCI LC-MS m/z 480.4 [MH+]

### EXAMPLE 106

### 6,7-diethoxy-4-{[2-ethyl-3-({[(1S)-2-hydroxy-1-phenylethyl]amino}methyl)phenyl]amino}quinoline-3-carboxamide bis(trifluoroacetate) (salt)

APCI LC-MS m/z 563.5 [MH+]

### EXAMPLE 107

### 6,7-diethoxy-4-[(2-ethyl-3-{[(2R)-2-(hydroxymethyl)pyrrolidin-1-yl]methyl}phenyl)amino]quinoline-3-carboxamide bis(trifluoroacetate) (salt)

APCI LC-MS m/z 529.5 [MH+]

### EXAMPLE 108

### 6,7-diethoxy-4-{[2-ethyl-3-({[(1S,2S)-2-hydroxy-1-(hydroxymethyl)-2-phenylethyl]amino}methyl)phenyl]amino}quinoline-3-carboxamide bis(trifluoroacetate) (salt)

APCI LC-MS m/z 493.5 [MH+]

### EXAMPLE 109

### 6,7-diethoxy-4-[(2-ethyl-3-{[(2-morpholin-4-ylethyl)amino]methyl}phenyl)amino]quinoline-3-carboxamide tris(trifluoroacetate)

APCI LC-MS m/z 559.5 [MH+]

### EXAMPLE 110

### 6,7-diethoxy-4-{[2-ethyl-3-({[(1R,2S)-2-hydroxy-2-(4-hydroxyphenyl)-1-methylethyl]amino}methyl)phenyl]amino}quinoline-3-carboxamide bis(trifluoroacetate) (salt)

APCI LC-MS m/z 522.4 [MH+]

### EXAMPLE 111

### 6,7-diethoxy-4-{[2-ethyl-3-({[(1R,2R)-2-hydroxy-1-(hydroxymethyl)-2-phenylethyl]amino}methyl)phenyl]amino}quinoline-3-carboxamide bis(trifluoroacetate) (salt)

APCI LC-MS m/z 559.5 [MH+]

### EXAMPLE 112

### 6,7-Diethoxy-4-{2-ethyl-3-[(2-hydroxy-1-hydroxymethyl-2-phenyl-ethylamino)-methyl]-phenylamino}-quinoline-3-carboxylic acid amide bis(trifluoroacetate)

APCI LC-MS m/z 559.5 [MH+]

### EXAMPLE 113

### 4-[(3-{[(2-cyanoethyl)amino]methyl}-2-ethylphenyl)amino]-6,7-diethoxyquinoline-3-carboxamide bis(trifluoroacetate)

APCI LC-MS m/z 462.5 [MH+]

### EXAMPLE 114

### 6,7-diethoxy-4-{[2-ethyl-3-({[1-(hydroxymethyl)-2-methylpropyl]amino}methyl)phenyl]amino}quinoline-3-carboxamide bis(trifluoroacetate) (salt)

APCI LC-MS m/z 495.5 [MH+]

### EXAMPLE 115

### 6,7-diethoxy-4-{[2-ethyl-3-({[4-(methylsulfonyl)benzyl]amino}methyl)phenyl]amino}quinoline-3-carboxamide bis(trifluoroacetate)

APCI LC-MS m/z 577.5 [MH+]

### EXAMPLE 116

### tert-butyl 3-{[3-(aminocarbonyl)-6,7-diethoxyquinolin-4-yl]amino}-2-ethylbenzylcarbamate

A mixture of 4-chloro-6,7-diethoxyquinoline-3-carboxamide (96.5 mg, 0.33 mmole), tert-butyl 3-amino-2-ethylbenzylcarbamate (prepared according to WO 02/092571) (119 mg, 0.476) in NMP (2 ml) was heated over night at 115 C. After cooling the solution was diluted with water and basified with NaHCO₃. The compound was extracted from the aqueous solution with ethylacetate (3×). The extracts were washed with water (2×), brine (2×), dried (Na₂SO₄), and evaporated. The residue was purified by silica chromatography (CH₂Cl₂/MeOH) to give 105 mg (62%) of the title compound as a white powder.

¹H NMR (299.946 MHz, DMSO-d₆) δ10.98 (1H, s), 8.86 (1H, s), 8.27 (1H, s), 7.58 (1H, s), 7.19 (1H, s), 7.00 (2H, mult), 6.56 (2H, mult), 4.22 (2H, d), 4.12 (4H, mult), 3.40 (1H, s), 2.81 (2H, d), 1.39 (9H, s), 1.33 (3H, t), 1.17 (3H, t), 1.02 (3H, t)

APCI-LC/MS m/z: 509.4 [MH+]

### EXAMPLE 117

### 4-{[3-(aminomethyl)-2-ethylphenyl]amino}-6,7-diethoxyquinoline-3-carboxamide

To a cooled solution of tert-butyl 3-{[3-(aminocarbonyl)-6,7-diethoxyquinolin-4-yl]amino}-2-ethylbenzylcarbamate (105 mg, 0.21 mmole) in CH₂Cl₂ (4 ml) was added TFA (4 ml). After 40 minutes at 0° C. the solvent was evaporated of. The residue was dissolved in CH₃CN/NH₃-aq solution and purified by preparative HPLC After freeze-drying (36 mg, 42%) of the title compound was obtained as a white powder.

¹H NMR (399.99 MHz, DMSO-d₆) δ 10.97 (1H, s), 8.85 (1H, s), 8.27 (1H, s), 7.57 (1H, s), 7.19 (1H, s), 7.17 (1H, s), 7.01 (1H, t), 6.63 (1H, s), 6.54 (1H, d), 4.13 (2H, q), 3.80 (2H, s), 2.81 (2H, q), 1.35 (3H, t), 1.18 (3H, t), 1.01 (3H, t)

APCI-LC/MS m/z: 409.2 [MH+]

### EXAMPLE 118

### 4-{[3-(aminomethyl)-2-methylphenyl]amino}-6,7-diethoxyquinoline-3-carboxamide

The title compound was prepared in an analogues way to example 117.

APCI-LC/MS m/z: 395.2 [MH+]

### EXAMPLE 119

### 6,7-diethoxy-4-({2-ethyl-3-[(L-tyrosylamino)methyl]phenyl}amino)quinoline-3-carboxamide bis(trifluoroacetate)

To a mixture of boc-L-tyrosine (45 mg, 0.16 mmol), HATU (61 mg, 0.16 mmol), and DIEA (26 mg, 0.2 mmol) in NMP/dichloromethane (1 ml) 4-{[3-(aminomethyl)-2-ethylphenyl]amino}-6,7-diethoxyquinoline-3-carboxamide (15 mg, 0.039 mmol) was added. The reaction was stirred for 1 h at room temperature. TFA (50% in DCM, 1 ml) was then added and the reaction mixture was stirred further for 1 h. The mixture was then diluted with water (1.0 ml) and purified by preparative HPLC using a gradient of acetonitrile/water at a flow rate of 20 ml/min. Freeze drying of the mixture afforded the title compound in 20% yield.

¹H NMR (400 MHz, DMSO-d₆) δ 9.39 (1H, s,); 8.98 (1H, s,); 8.85 (1H, s,); 8.54 (1H, s,); 8.20 (3H, s,); 8.02 (1H, s,); 7.28 (1H, s,); 7.16 (1H, s,); 7.04 (2H, d, J=8.5 Hz); 6.72 (2H, d, J=8.5 Hz); 6.65 (1H, s,); 4.49 (1H, s,); 4.34 (1H, d, J=11.3 Hz); 4.20 (2H, q, J=7.0 Hz); 3.99 (1H, s,); 2.96 (2H, t, J=6.7 Hz); 2.70 (1H, d, J=27.3 Hz); 1.39 (3H, t, J=7.0 Hz); 1.13 (6H, t, J=7.5 Hz); 1.07 (9H, t, J=6.7 Hz).

APCI-LC/MS m/z: 572.6 [MH+]

The title compounds of examples 120-183 were prepared in analogous manner to example 119 using 4-{[3-(aminomethyl)-2-ethylphenyl]amino}-6,7-diethoxyquinoline-3-carboxamide, 4-{[3-(aminomethyl)-2-ethylphenyl]amino}-6,7-dimetoxyquinoline-3-carboxamide or 4-{[3-(aminomethyl)-2-methylphenyl]amino}-6,7-dimetoxyquinoline-3-carboxamide and an appropriate amino acid, acid chloride or isocyanate.

### EXAMPLE 120

### 6,7-diethoxy-4-{[3-({[(ethylamino)carbonyl]amino}methyl)-2-methylphenyl]amino}quinoline-3-carboxamide

APCI LC-MS m/z: 466.5 [MH+]

### EXAMPLE 121

### 4-({3-[(acetylamino)methyl]-2-methylphenyl}amino)-6,7-diethoxyquinoline-3-carboxamide

APCI LC-MS m/z: 437.4 [MH+]

### EXAMPLE 122

### 6,7-diethoxy-4-({2-methyl-3-[({[(4-methyl-2,5-dioxoimidazolidin-4-yl)methyl]sulfonyl}amino)methyl]phenyl}amino)quinoline-3-carboxamide

APCI LC-MS m/z: 585.1 [MH+]

### EXAMPLE 123

### 4-({3-[(acetylamino)methyl]-2-ethylphenyl}amino)-6,7-dimethoxyquinoline-3-carboxamide

APCI LC-MS m/z: 423.4 [MH+]

### EXAMPLE 124

### 4-{[2-ethyl-3-({[(ethylamino)carbonyl]amino}methyl)phenyl]amino}-6,7-dimethoxyquinoline-3-carboxamide

APCI LC-MS m/z: 585.1 [MH+]

### EXAMPLE 125

### 4-[(2-ethyl-3-{[(methylsulfonyl)amino]methyl}phenyl)amino]-6,7-dimethoxyquinoline-3-carboxamide

APCI LC-MS m/z: 459.2 [MH+]

### EXAMPLE 126

### 4-({2-ethyl-3-[(L-valylamino)methyl]phenyl}amino)-6,7-dimethoxyquinoline-3-carboxamide

APCI LC-MS m/z: 480.2 [MH+]

### EXAMPLE 127

### 4-[(3-{[(3-cyclohexyl-L-alanyl)amino]methyl}-2-ethylphenyl)amino]-6,7-dimethoxyquinoline-3-carboxamide

APCI LC-MS m/z: 534.5 [MH+]

### EXAMPLE 128

### 6,7-diethoxy-4-({2-ethyl-3-[(L-methionylamino)methyl]phenyl}amino)quinoline-3-carboxamide bis(trifluoroacetate)

APCI LC-MS m/z: 540.5 [MH+]

### EXAMPLE 129

### 6,7-diethoxy-4-({2-ethyl-3-[(L-prolylamino)methyl]phenyl}amino)quinoline-3-carboxamide bis(trifluoroacetate)

APCI LC-MS m/z: 506.4 [MH+]

### EXAMPLE 130

### 6,7-diethoxy-4-({2-ethyl-3-[(L-threonylamino)methyl]phenyl}amino)quinoline-3-carboxamide bis(trifluoroacetate)

APCI LC-MS m/z: 510.4 [MH+]

### EXAMPLE 131

### N˜1˜-(3-{[3-(aminocarbonyl)-6,7-diethoxyquinolin-4-yl]amino}-2-ethylbenzyl)-L-alpha-glutamine bis(trifluoroacetate)

APCI LC-MS m/z: 538.5 [MH+]

### EXAMPLE 132

### 6,7-diethoxy-4-({2-ethyl-3-[(L-valylamino)methyl]phenyl}amino)quinoline-3-carboxamide bis(trifluoroacetate)

APCI LC-MS m/z: 508.5 [MH+]

### EXAMPLE 133

### 4-({3-[(L-arginylamino)methyl]-2-ethylphenyl}amino)-6,7-diethoxyquinoline-3-carboxamide tris(trifluoroacetate)

APCI LC-MS m/z: 565.6 [MH+]

### EXAMPLE 134

### 4-({3-[(L-alanylamino)methyl]-2-ethylphenyl}amino)-6,7-diethoxyquinoline-3-carboxamide bis(trifluoroacetate)

APCI LC-MS m/z: 480.4 [MH+]

### EXAMPLE 135

### 6,7-diethoxy-4-({2-ethyl-3-[(D-serylamino)methyl]phenyl}amino)quinoline-3-carboxamide bis(trifluoroacetate)

APCI LC-MS m/z: 496.4 [MH+]

### EXAMPLE 136

### 4-[(3-{[(3-cyclohexyl-L-alanyl)amino]methyl}-2-ethylphenyl)amino]-6,7-diethoxyquinoline-3-carboxamide bis(trifluoroacetate)

APCI LC-MS m/z: 562.5 [MH+]

### EXAMPLE 137

### 6,7-diethoxy-4-{[2-ethyl-3-({[(4S)-1,3-thiazolidin-4-ylcarbonyl]amino}methyl)phenyl]amino}quinoline-3-carboxamide bis(trifluoroacetate)

APCI LC-MS m/z: 524.4 [MH+]

### EXAMPLE 138

### 6,7-diethoxy-4-{[2-ethyl-3-({[(4R)-4-hydroxy-L-prolyl]amino}methyl)phenyl]amino}quinoline-3-carboxamide bis(trifluoroacetate) (salt)

APCI LC-MS m/z: 522.5 [MH+]

### EXAMPLE 139

### 6,7-diethoxy-4-({2-ethyl-3-[(D-leucylamino)methyl]phenyl}amino)quinoline-3-carboxamide bis(trifluoroacetate)

APCI LC-MS m/z: 522.5 [MH+]

### EXAMPLE 140

### N˜1˜-(3-{[3-(aminocarbonyl)-6,7-diethoxyquinolin-4-yl]amino}-2-ethylbenzyl)-L-aspartamide bis(trifluoroacetate)

APCI LC-MS m/z: 523.2 [MH+]

### EXAMPLE 141

### 6,7-diethoxy-4-{[2-ethyl-3-({[(2S)-piperidin-2-ylcarbonyl]amino}methyl)phenyl]amino}quinoline-3-carboxamide bis(trifluoroacetate)

APCI LC-MS m/z: 520.5 [MH+]

### EXAMPLE 142

### 4-[(3-{[(3-cyclohexyl-D-alanyl)amino]methyl}-2-ethylphenyl)amino]-6,7-diethoxyquinoline-3-carboxamide bis(trifluoroacetate)

APCI LC-MS m/z: 562.5 [MH+]

### EXAMPLE 143

### 6,7-diethoxy-4-{[2-ethyl-3-({[(2R)-piperidin-2-ylcarbonyl]amino}methyl)phenyl]amino}quinoline-3-carboxamide bis(trifluoroacetate)

APCI LC-MS m/z 520.5 [MH+]

### EXAMPLE 144

### 4-{[3-({[(2S)-2-aminopent-4-enoyl]amino}methyl)-2-ethylphenyl]amino}-6,7-diethoxyquinoline-3-carboxamide bis(trifluoroacetate)

APCI LC-MS m/z: 506.5 [MH+]

### EXAMPLE 145

### 4-{[3-({[(2S)-azetidin-2-ylcarbonyl]amino}methyl)-2-ethylphenyl]amino}-6,7-diethoxyquinoline-3-carboxamide bis(trifluoroacetate)

APCI LC-MS m/z: 492.4 [MH+]

### EXAMPLE 146

### 6,7-diethoxy-4-[(2-ethyl-3-{[(5-methyl-L-norleucyl)amino]methyl}phenyl)amino]quinoline-3-carboxamide bis(trifluoroacetate)

APCI LC-MS m/z: 536.5 [MH+]

### EXAMPLE 147

### 6,7-diethoxy-4-{[2-ethyl-3-({[(4R)-1,3-thiazolidin-4-ylcarbonyl]amino}methyl)phenyl]amino}quinoline-3-carboxamide bis(trifluoroacetate)

APCI LC-MS m/z: 524.4 [MH+]

### EXAMPLE 148

### 6,7-diethoxy-4-[(2-ethyl-3-{[(4-nitro-D-phenylalanyl)amino]methyl}phenyl)amino]quinoline-3-carboxamide bis(trifluoroacetate)

APCI LC-MS m/z: 601.5 [MH+]

### EXAMPLE 149

### 4-{[3-({[(1-amino-2,3-dihydro-1H-inden-1-yl)carbonyl]amino}methyl)-2-ethylphenyl]amino}-6,7-diethoxyquinoline-3-carboxamide bis(trifluoroacetate)

APCI LC-MS m/z: 568.5 [MH+]

### EXAMPLE 150

### 4-{[3-({[(1-aminocyclohexyl)carbonyl]amino}methyl)-2-ethylphenyl]amino}-6,7-diethoxyquinoline-3-carboxamide bis(trifluoroacetate)

APCI LC-MS m/z: 534.5 [MH+]

### EXAMPLE 151

### 6,7-diethoxy-4-{[2-ethyl-3-({[(3R)-1,2,3,4-tetrahydroisoquinolin-3-ylcarbonyl]amino}methyl)phenyl]amino}quinoline-3-carboxamide bis(trifluoroacetate)

APCI LC-MS m/z: 568.5 [MH+]

### EXAMPLE 152

### 4-{[3-({[(2R)-2-amino-4-phenylbutanoyl]amino}methyl)-2-ethylphenyl]amino}-6,7-diethoxyquinoline-3-carboxamide bis(trifluoroacetate)

APCI LC-MS m/z: 570.5 [MH+]

### EXAMPLE 153

### 6,7-diethoxy-4-{[2-ethyl-3-({[(3S)-1,2,3,4-tetrahydroisoquinolin-3-ylcarbonyl]amino}methyl)phenyl]amino}quinoline-3-carboxamide bis(trifluoroacetate)

APCI LC-MS m/z: 568.5 [MH+]

### EXAMPLE 154

### 6,7-diethoxy-4-[(2-ethyl-3-{[(4-piperidin-4-yl-L-prolyl)amino]methyl}phenyl)amino]quinoline-3-carboxamide tris(trifluoroacetate)

APCI LC-MS m/z: 589.6 [MH+]

### EXAMPLE 155

### 4-[(3-{[(3-amino-L-alanyl)amino]methyl}-2-ethylphenyl)amino]-6,7-diethoxyquinoline-3-carboxamide tris(trifluoroacetate)

APCI LC-MS m/z: 495.4 [MH+]

### EXAMPLE 156

### 6,7-diethoxy-4-({2-ethyl-3-[(D-phenylalanylamino)methyl]phenyl}amino)quinoline-3-carboxamide bis(trifluoroacetate)

APCI LC-MS m/z: 556.5 [MH+]

### EXAMPLE 157

### 4-{[3-({[(2S)-2-amino-4-phenylbutanoyl]amino}methyl)-2-ethylphenyl]amino}-6,7-diethoxyquinoline-3-carboxamide bis(trifluoroacetate)

APCI LC-MS m/z: 570.5 [MH+]

### EXAMPLE 158

### 6,7-diethoxy-4-{[2-ethyl-3-({[(3S)-piperidin-3-ylcarbonyl]amino}methyl)phenyl]amino}quinoline-3-carboxamide bis(trifluoroacetate)

APCI LC-MS m/z: 520.5 [MH+]

### EXAMPLE 159

### 6,7-diethoxy-4-{[2-ethyl-3-({[(3R)-piperidin-3-ylcarbonyl]amino}methyl)phenyl]amino}quinoline-3-carboxamide bis(trifluoroacetate)

APCI LC-MS m/z: 520.5 [MH+]

### EXAMPLE 160

### 4-{[3-({[(2S)-2-amino-2-phenylacetyl]amino}methyl)-2-ethylphenyl]amino}-6,7-diethoxyquinoline-3-carboxamide bis(trifluoroacetate)

APCI LC-MS m/z: 542.5 [MH+]

### EXAMPLE 161

### 6,7-diethoxy-4-({2-ethyl-3-[(L-leucylamino)methyl]phenyl}amino)quinoline-3-carboxamide bis(trifluoroacetate)

APCI LC-MS m/z: 522.5 [MH+]

### EXAMPLE 162

### 6,7-diethoxy-4-({2-ethyl-3-[(D-prolylamino)methyl]phenyl}amino)quinoline-3-carboxamide bis(trifluoroacetate)

APCI LC-MS m/z: 506.5 [MH+]

### EXAMPLE 163

### 4-{[3-({[(2S)-2,5-dihydro-1H-pyrrol-2-ylcarbonyl]amino}methyl)-2-ethylphenyl]amino}-6,7-diethoxyquinoline-3-carboxamide bis(trifluoroacetate)

APCI LC-MS m/z: 504.4 [MH+]

### EXAMPLE 164

### 6,7-diethoxy-4-({2-ethyl-3-[(glycylamino)methyl]phenyl}amino)quinoline-3-carboxamide bis(trifluoroacetate)

APCI LC-MS m/z: 466.4 [MH+]

### EXAMPLE 165

### 4-{[3-({[2-amino-4-(methylsulfinyl)butanoyl]amino}methyl)-2-ethylphenyl]amino}-6,7-diethoxyquinoline-3-carboxamide bis(trifluoroacetate)

APCI LC-MS m/z: 556.5 [MH+]

### EXAMPLE 166

### 6,7-diethoxy-4-{[2-ethyl-3-({[3-(2-furyl)-L-alanyl]amino}methyl)phenyl]amino}quinoline-3-carboxamide bis(trifluoroacetate)

APCI LC-MS m/z: 546.5 [MH+]

### EXAMPLE 167

### 6,7-diethoxy-4-[(2-ethyl-3-{[(3-pyridin-2-yl-L-alanyl)amino]methyl}phenyl)amino]quinoline-3-carboxamide tris(trifluoroacetate)

APCI LC-MS m/z: 557.5 [M+]

### EXAMPLE 168

### 6,7-diethoxy-4-{[2-ethyl-3-({[3-(2-thienyl)-L-alanyl]amino}methyl)phenyl]amino}quinoline-3-carboxamide bis(trifluoroacetate)

APCI LC-MS m/z: 562.4 [MH+]

### EXAMPLE 169

### 6,7-diethoxy-4-{[2-ethyl-3-({[3-(1,3-thiazol-4-yl)-L-alanyl]amino}methyl)phenyl]amino}quinoline-3-carboxamide tris(trifluoroacetate)

APCI LC-MS m/z: 563.5 [MH+]

### EXAMPLE 170

### 4-{[3-({[(2S)-2-amino-2-cyclopentylacetyl]amino}methyl)-2-ethylphenyl]amino}-6,7-diethoxyquinoline-3-carboxamide bis(trifluoroacetate)

APCI LC-MS m/z: 534.5 [MH+]

### EXAMPLE 171

### 4-{[3-({[(2S)-2-aminopent-4-ynoyl]amino}methyl)-2-ethylphenyl]amino}-6,7-diethoxyquinoline-3-carboxamide bis(trifluoroacetate)

APCI LC-MS m/z: 504.4 [MH+]

### EXAMPLE 172

### 6,7-diethoxy-4-({2-ethyl-3-[(L-norvalylamino)methyl]phenyl}amino)quinoline-3-carboxamide bis(trifluoroacetate)

APCI LC-MS m/z: 508.5 [MH+]

### EXAMPLE 173

### 4-{[3-({[(2R)-2-amino-2-phenylacetyl]amino}methyl)-2-ethylphenyl]amino}-6,7-diethoxyquinoline-3-carboxamide bis(trifluoroacetate)

APCI LC-MS m/z: 542.5 [MH+]

### EXAMPLE 174

### 6,7-diethoxy-4-{[2-ethyl-3-({[(4R)-4-hydroxy-D-prolyl]amino}methyl)phenyl]amino}quinoline-3-carboxamide bis(trifluoroacetate) (salt)

APCI LC-MS m/z: 522.4 [MH+]

### EXAMPLE 175

### 4-({3-[(beta-alanylamino)methyl]-2-ethylphenyl}amino)-6,7-diethoxyquinoline-3-carboxamide bis(trifluoroacetate)

APCI LC-MS m/z: 480.4 [MH+]

### EXAMPLE 176

### 6,7-diethoxy-4-[(2-ethyl-3-{[(3-pyridin-3-yl-L-alanyl)amino]methyl}phenyl)amino]quinoline-3-carboxamide tris(trifluoroacetate)

APCI LC-MS m/z: 557.5 [MH+]

### EXAMPLE 177

### 6,7-diethoxy-4-[(2-ethyl-3-{[(3-pyridin-3-yl-D-alanyl)amino]methyl}phenyl)amino]quinoline-3-carboxamide tris(trifluoroacetate)

APCI LC-MS m/z: 557.5 [MH+]

### EXAMPLE 178

### 4-{[3-({[N˜5˜-(aminocarbonyl)-L-ornithyl]amino}methyl)-2-ethylphenyl]amino}-6,7-diethoxyquinoline-3-carboxamide bis(trifluoroacetate)

APCI LC-MS m/z: 566.5 [MH+]

### EXAMPLE 179

### 6,7-diethoxy-4-[(2-ethyl-3-{[(5-methyl-D-norleucyl)amino]methyl}phenyl)amino]quinoline-3-carboxamide bis(trifluoroacetate)

APCI LC-MS m/z: 536.5 [MH+]

### EXAMPLE 180

### 4-[(3-{[(2,3-dihydro-1H-isoindol-1-ylcarbonyl)amino]methyl}-2-ethylphenyl)amino]-6,7-diethoxyquinoline-3-carboxamide bis(trifluoroacetate)

APCI LC-MS m/z: 554.5 [MH+]

### EXAMPLE 181

### 6,7-diethoxy-4-({2-ethyl-3-[(L-isoleucylamino)methyl]phenyl}amino)quinoline-3-carboxamide bis(trifluoroacetate)

APCI LC-MS m/z: 522.5 [MH+]

### EXAMPLE 182

### 6,7-diethoxy-4-({2-ethyl-3-[(D-valylamino)methyl]phenyl}amino)quinoline-3-carboxamide bis(trifluoroacetate)

APCI LC-MS m/z: 508.5 [MH+]

### EXAMPLE 183

### 4-{[3-({[(1-aminocyclopentyl)carbonyl]amino}methyl)-2-ethylphenyl]amino}-6,7-diethoxyquinoline-3-carboxamide bis(trifluoroacetate)

APCI LC-MS m/z: 520.5 [MH+]

### EXAMPLE 184

### 4-{[2-ethyl-3-(hydroxymethyl)phenyl]amino}-7-{3-[isobutyryl(isopropyl)amino]propoxy}-6-methoxyquinoline-3-carboxamide

To a solution of 4-{[2-ethyl-3-(hydroxymethyl)phenyl]amino}-7-[3-(isopropylamino)propoxy]-6-methoxyquinoline-3-carboxamide, prepared according to the procedure described in WO 02/092571, 10 mg, 0.021 mmole), triethylamine (0.2 ml) in NMP (1 ml) was added isobutiric anhydride (3.8 mg, 0.024 mmole). The mixture was stirred overnight at ambient temperature. The solution was diluted with water and the product was purified by preparative HPLC. After freeze-drying 6.7 mg (59%) was obtained of the title compound as a white powder.

¹H NMR (399.99 MHz, DMSO-d₆) δ 11.03 (1H, s), 8.87 (1H, s), 8.28 (1H, s), 7.60 (1H, s), 7.21 (2H, mult), 7.04 (1H, t), 6.65 (1H, d), 6.59 (1H, d), 5.16 (1H, t), 4.60 (2H, d), 4.46 (1H, quintet), 4.13 (2H, mult), 3.23 (1H, t), 3.21 (3H, d), 2.81 (3H, mult), 1.96 (2H, s), 1.19 (3H, t), 1.12 (3H, t), 1.05 (3H, d), 0.98 (3H, d), 0.90 (3H, d)

APCI-LC/MS m/z: 537.3 [MH+]

Following examples 185-202 were prepared in analogous manner to example 184 using the appropriate anhydride, acid chloride or isocyanate.

### EXAMPLE 185

### 7-{3-[acetyl(isopropyl)amino]propoxy}-4-{[2-ethyl-3-(hydroxymethyl)phenyl]amino}-6-methoxyquinoline-3-carboxamide

¹H NMR (399.99 MHz, DMSO-d₆) δ 11.07 (1H, s), 8.87 (1H, s), 8.29 (1H, s), 7.61 (1H, s), 7.24 (1H, s), 7.19 (2H, d), 7.05 (1H, t), 6.65 (1H, d), 6.60 (1H, d), 5.16 (1H, t), 4.59 (2H, d), 4.44 (0.5H, t), 4.12 (2H, dt), 4.00 (0.5H, quintet), 3.23 (1H, t), 3.21 (3H, d), 2.79 (2H, q), 1.97 (5H, mult), 1.19 (3H, t), 1.08 (6H, mult)

APCI LC-MS m/z: 509.3 [MH+]

### EXAMPLE 186

### 6-[2-(acetylamino)ethoxy]-4-[(2-ethylphenyl)amino]-7-methoxyquinoline-3-carboxamide

APCI LC-MS m/z: 423.2 [MH+]

### EXAMPLE 187

### 6-{2-[acetyl(methyl)amino]ethoxy}-4-[(2-ethylphenyl)amino]-7-methoxyquinoline-3-carboxamide

APCI LC-MS m/z: 437.2 [MH+]

### EXAMPLE 188

### 6-{2-[acetyl(isopropyl)amino]ethoxy}-4-[(2-ethylphenyl)amino]-7-methoxyquinoline-3-carboxamide

APCI LC-MS m/z: 465.2 [MH+]

### EXAMPLE 189

### 4-[(2-ethylphenyl)amino]-6-{2-[isobutyryl(methyl)amino]ethoxy}-7-methoxyquinoline-3-carboxamide

APCI LC-MS m/z: 465.2 [MH+]

### EXAMPLE 190

### 4-[(2-ethylphenyl)amino]-6-{2-[isobutyryl(isopropyl)amino]ethoxy}-7-methoxyquinoline-3-carboxamide

APCI LC-MS m/z: 493.3 [MH+]

### EXAMPLE 191

### 7-{3-[acetyl(methyl)amino]propoxy}-4-{[2-ethyl-3-(hydroxymethyl)phenyl]amino}-6-methoxyquinoline-3-carboxamide

APCI LC-MS m/z: 481.5 [MH+]

### EXAMPLE 192

### 4-{[2-ethyl-3-(hydroxymethyl)phenyl]amino}-7-{3-[isobutyryl(methyl)amino]propoxy}-6-methoxyquinoline-3-carboxamide

APCI LC-MS m/z: 509.6 [MH+]

### EXAMPLE 193

### 7-{3-[acetyl(cyclopropyl)amino]propoxy}-4-{[2-ethyl-3-(hydroxymethyl)phenyl]amino}-6-methoxyquinoline-3-carboxamide

APCI LC-MS m/z: 507.6 [MH+]

### EXAMPLE 194

### 7-{3-[cyclopropyl(isobutyryl)amino]propoxy}-4-{[2-ethyl-3-(hydroxymethyl)phenyl]amino}-6-methoxyquinoline-3-carboxamide

APCI LC-MS m/z: 534.7 [MH+]

### EXAMPLE 195

### 7-[3-(acetylamino)propoxy]-4-{[2-ethyl-3-(hydroxymethyl)phenyl]amino}-6-methoxyquinoline-3-carboxamide

APCI LC-MS m/z: 467.3 [MH+]

### EXAMPLE 196

### 4-{[2-ethyl-3-(hydroxymethyl)phenyl]amino}-7-[3-(isobutyrylamino)propoxy]-6-methoxyquinoline-3-carboxamide

APCI LC-MS m/z: 494.7 [MH+]

### EXAMPLE 197

### 6-{2-[(cyclopropylcarbonyl)(methyl)amino]ethoxy}-4-[(2-ethylphenyl)amino]-7-methoxyquinoline-3-carboxamide

APCI LC-MS m/z: 463.2 [MH+]

### EXAMPLE 198

### 6-{2-[(cyclopropylcarbonyl)(isopropyl)amino]ethoxy}-4-[(2-ethylphenyl)amino]-7-methoxyquinoline-3-carboxamide

APCI LC-MS m/z: 491.2 [MH+]

### EXAMPLE 199

### 4-{[2-ethyl-3-(hydroxymethyl)phenyl]amino}-7-{3-[isopropyl(methylsulfonyl)amino]propoxy}-6-methoxyquinoline-3-carboxamide

APCI LC-MS m/z: 545.3 [MH+]

### EXAMPLE 200

### 4-{[2-ethyl-3-(hydroxymethyl)phenyl]amino}-6-methoxy-7-{3-[(methylsulfonyl)amino]propoxy}quinoline-3-carboxamide

APCI LC-MS m/z: 503.6 [MH+]

### EXAMPLE 201

### tert-butyl {3-[(3-(aminocarbonyl)-4-{[2-ethyl-3-(hydroxymethyl)phenyl]amino}-6-methoxyquinolin-7-yl)oxy]propyl}isopropylcarbamate

¹H NMR (399.99 MHz, CD₃OD) δ 8.79 (s, 1H), 7.28 (d, J=7.2 Hz, 1H), 7.20 (s, 1H), 7.12 (t, J=7.8 Hz, 1H), 6.81-6.74 (m, 2H), 4.75 (s, 2H), 4.16 (t, J=5.9 Hz, 3H), 3.36-3.31 (m, 2H), 3.28 (s, 3H), 2.94 (q, J=7.4 Hz, 2H), 2.14-2.04 (m, 2H), 1.42 (s, 9H), 1.30 (t, J=7.5 Hz, 3H), 1.16 (d, J=6.8 Hz, 6H).

APCI LC-MS m/z: 567.3 [MH+]

### EXAMPLE 202

### 4-{[2-ethyl-3-(hydroxymethyl)phenyl]amino}-7-(3-{isopropyl [(isopropylamino)carbonyl]amino}propoxy)-6-methoxyquinoline-3-carboxamide

¹H NMR (399.99 MHz, DMSO-d₆) δ 11.04 (s, 1H), 8.88 (s, 1H), 8.29 (s, 1H), 7.61 (s, 1H), 7.24 (s, 1H), 7.19 (d, J=7.2 Hz, 1H), 7.05 (t, J=7.7 Hz, 1H), 6.66 (s, 1H), 6.60 (d, J=7.6 Hz, 1H), 5.61 (d, J=7.8 Hz, 1H), 5.17 (t, J=5.4 Hz, 1H), 4.61 (d, J=5.4 Hz, 2H), 4.22 (quintet, J=6.7 Hz, 1H), 4.12 (t, J=6.2 Hz, 2H), 3.76 (quintet, J=6.8 Hz, 1H), 3.22 (s, 3H), 3.18 (t, J=7.3 Hz, 2H), 2.80 (q, J=7.5 Hz, 2H), 1.99-1.87 (m, 2H), 1.20 (t, J=7.4 Hz, 3H), 1.06-0.97 (m, 12H)

APCI LC-MS m/z: 552.3 [MH+]

Following examples 203-233 are prepared according to the procedure described in WO 02/092571

### EXAMPLE 203

### 7-[3-(cyclopropylamino)propoxy]-4-{[2-ethyl-3-(hydroxymethyl)phenyl]amino}-6-methoxyquinoline-3-carboxamide

APCI LC-MS m/z: 465.4 [MH+]

### EXAMPLE 204

### 6-[3-(cyclopropylamino)propoxy]-4-{[2-ethyl-3-(hydroxymethyl)phenyl]amino}-7-methoxyquinoline-3-carboxamide

¹H NMR (399.99 MHz, DMSO-d₆): δ 11.02 (1H, s); 8.88 (1H, s); 8.29 (1H, br s); 7.61 (1H, br s); 7.23 (1H, s); 7.18 (1H, d, J=7.4 Hz); 7.04 (1H, t, J=7.8 Hz); 6.65 (1H, s); 6.60 (1H, d, J=7.7 Hz); 5.19 (1H, br s); 4.61 (2H, s); 3.88 (3H, s); 3.37 (2H, s); 2.80 (2H, q, J=7.4 Hz); 1.99 (1H, dquintet, J=6.7, 3.4 Hz); 1.57 (2H, quintet, J=6.6 Hz); 1.20 (3H, t, J=7.5 Hz); 0.34 (2H, td, J=6.4, 4.2 Hz); 0.15 (2H, dt, J=6.1, 3.7 Hz).

APCI-LC/MS m/z: 465.4 [MH+]

### EXAMPLE 205

### 7-{3-[(2-cyanoethyl)(methyl)amino]propoxy}-4-{[3-(hydroxymethyl)-2-methylphenyl]amino}-6-methoxyquinoline-3-carboxamide bis(trifluoroacetate) (salt)

APCI LC-MS m/z: 478.3 [MH+]

### EXAMPLE 206

### 4-{[3-(hydroxymethyl)-2-methylphenyl]amino}-6-methoxy-7-[3-(2-methylpiperidin-1-yl)propoxy]quinoline-3-carboxamide

APCI LC-MS m/z: 493.3 [MH+]

### EXAMPLE 207

### 7-{3-[(2-cyanoethyl)(methyl)amino]propoxy}-4-{[3-(hydroxymethyl)-2-methylphenyl]amino}-6-methoxyquinoline-3-carboxamide

APCI LC-MS m/z: 478.2 [MH+]

### EXAMPLE 208

### 4-{[3-(hydroxymethyl)-2-methylphenyl]amino}-7-[3-(3-hydroxypiperidin-1-yl)propoxy]-6-methoxyquinoline-3-carboxamide

APCI LC-MS m/z: 495.3 [MH+]

### EXAMPLE 209

### 4-{[3-(hydroxymethyl)-2-methylphenyl]amino}-7-[3-(4-hydroxypiperidin-1-yl)propoxy]-6-methoxyquinoline-3-carboxamide

APCI LC-MS m/z: 495.3 [MH+]

### EXAMPLE 210

### 6-methoxy-4-[(2-methylphenyl)amino]-7-[3-(2-methylpiperidin-1-yl)propoxy]quinoline-3-carboxamide

APCI LC-MS m/z: 463.3 [MH+]

### EXAMPLE 211

### 7-[3-(3-hydroxypiperidin-1-yl)propoxy]-6-methoxy-4-[(2-methylphenyl)amino]quinoline-3-carboxamide

APCI LC-MS m/z: 465.3 [MH+]

### EXAMPLE 212

### 7-[3-(4-hydroxypiperidin-1-yl)propoxy]-6-methoxy-4-[(2-methylphenyl)amino]quinoline-3-carboxamide

APCI LC-MS m/z: 465.3 [MH+]

### EXAMPLE 213

### 4-{[3-(hydroxymethyl)-2-methylphenyl]amino}-7-[3-(3-hydroxypyrrolidin-1-yl)propoxy]-6-methoxyquinoline-3-carboxamide

APCI LC-MS m/z: 481.1 [MH+]

### EXAMPLE 214

### 4-{[2-ethyl-3-(hydroxymethyl)phenyl]amino}-6-methoxy-7-[3-(1H-1,2,4-triazol-1-yl)propoxy]quinoline-3-carboxamide bis(trifluoroacetate) (salt)

APCI LC-MS m/z: 477.6 [MH+]

### EXAMPLE 215

### 7-[2-(cyclopropylamino)ethoxy]-4-{[3-(hydroxymethyl)-2-methylphenyl]amino}-6-methoxyquinoline-3-carboxamide bis(trifluoroacetate) (salt)

APCI LC-MS m/z: 437.5 [MH+]

### EXAMPLE 216

### 6-[2-(cyclopropylamino)ethoxy]-4-{[3-(hydroxymethyl)-2-methylphenyl]amino}-7-methoxyquinoline-3-carboxamide bis(trifluoroacetate) (salt)

APCI LC-MS m/z: 437.2 [MH+]

### EXAMPLE 217

### 6-[2-(cyclopropylamino)ethoxy]-4-[(4-ethylphenyl)amino]-7-methoxyquinoline-3-carboxamide

APCI LC-MS m/z: 421.5 [MH+]

### EXAMPLE 218

### 6-[2-(cyclopropylamino)ethoxy]-4-[(3-ethylphenyl)amino]-7-methoxyquinoline-3-carboxamide

APCI LC-MS m/z: 421.5 [MH+]

### EXAMPLE 219

### 6-[2-(cyclopropylamino)ethoxy]-7-methoxy-4-[(2-methylphenyl)amino]quinoline-3-carboxamide bis(trifluoroacetate)

APCI LC-MS m/z: 407.2 [MH+]

### EXAMPLE 220

### 6-{2-[(2-cyanoethyl)amino]ethoxy}-4-{[3-(hydroxymethyl)-2-methylphenyl]amino}-7-methoxyquinoline-3-carboxamide bis(trifluoroacetate) (salt)

APCI LC-MS m/z: 450.2 [MH+]

### EXAMPLE 221

### 6-[3-(cyclopropylamino)propoxy]-4-[(2-ethylphenyl)amino]-7-methoxyquinoline-3-carboxamide bis(trifluoroacetate)

APCI LC-MS m/z: 435.3 [MH+]

### EXAMPLE 222

### 6-{3-[(cyanomethyl)amino]propoxy}-4-[(2-ethylphenyl)amino]-7-methoxyquinoline-3-carboxamide

APCI LC-MS m/z: 434.3 [MH+]

### EXAMPLE 223

### 6-[3-(Carbamoylmethyl-amino)-propoxy]-4-(2-ethyl-phenylamino)-7-methoxy-quinoline-3-carboxylic acid amide bis(trifluoroacetate)

APCI LC-MS m/z: 452.3 [MH+]

### EXAMPLE 224

### methyl N-[3-({3-(aminocarbonyl)-4-[(2-ethylphenyl)amino]-7-methoxyquinolin-6-yl}oxy)propyl]glycinate bis(trifluoroacetate)

APCI LC-MS m/z: 467.3 [MH+]

### EXAMPLE 225

### 7-(3-cyanopropoxy)-4-{[2-ethyl-3-(hydroxymethyl)phenyl]amino}-6-methoxyquinoline-3-carboxamide trifluoroacetate (salt)

APCI LC-MS m/z: 435.2 [MH+]

### EXAMPLE 226

### 2-[(3-(aminocarbonyl)-4-{[2-ethyl-3-(hydroxymethyl)phenyl]amino}-6-methoxyquinolin-7-yl)oxy]ethyl acetate trifluoroacetate (salt)

APCI LC-MS m/z: 568.5 [MH+]

### EXAMPLE 227

### 6-[2-(cyclopropylamino)ethoxy]-4-[(2-ethylphenyl)amino]-7-methoxyquinoline-3-carboxamide

APCI LC-MS m/z: 421.1 [MH+]

### EXAMPLE 228

### 7-[3-(2,5-dioxopyrrolidin-1-yl)propoxy]-4-{[2-ethyl-3-(hydroxymethyl)phenyl]amino}-6-methoxyquinoline-3-carboxamide

APCI LC-MS m/z: 507.6 [MH+]

### EXAMPLE 229

### 4-{[2-ethyl-3-(hydroxymethyl)phenyl]amino}-6-methoxy-7-[3-(3-methyl-2,5-dioxoimidazolidin-1-yl)propoxy]quinoline-3-carboxamide

APCI LC-MS m/z: 522.6 [MH+]

### EXAMPLE 230

### 4-{[2-ethyl-3-(hydroxymethyl)phenyl]amino}-6-methoxy-7-[3-(3,4,4-trimethyl-2,5-dioxoimidazolidin-1-yl)propoxy]quinoline-3-carboxamide

APCI LC-MS m/z: 550.5 [MH+]

### EXAMPLE 231

### 7-(cyclopentyloxy)-4-{[2-ethyl-3-(hydroxymethyl)phenyl]amino}-6-methoxyquinoline-3-carboxamide

APCI LC-MS m/z: 436.2 [MH+]

### EXAMPLE 232

### 6-(cyclopentyloxy)-4-[(2-ethylphenyl)amino]-7-methoxyquinoline-3-carboxamide

APCI LC-MS m/z: 406.5 [MH+]

### EXAMPLE 233

### 1-{3-[(3-(aminocarbonyl)-4-{[3-(hydroxymethyl)-2-methylphenyl]amino}-6-methoxyquinolin-7-yl)oxy]propyl}-1-methylpyrrolidinium iodide

To a mixture of 7-(3-chloropropoxy)-4-{[3-(hydroxymethyl)-2-methylphenyl]amino}-6-methoxyquinoline-3-carboxamide (0.050 g, 0.116 mmol) in aceton (4.0 ml) 1-methylpyrrolidine (0.040 g, 0.46 mmol) and sodium iodide was added and the mixture heated to 60° C. for 24 h. After cooling, aceton was evaporated, the reaction mixture was diluted with water (2.0 ml) and purified by preparative HPLC using a gradient of acetonitrile/water at a flow rate of 20 ml/min. Freeze drying of the mixture afforded the title compound.

¹H NMR (400 MHz, CD₃OD) δ 8.80 (1H, s,); 7.27 (1H, d, J=7.2 Hz); 7.22 (1H, s,); 7.14 (1H, t, J=7.6 Hz); 6.86 (1H, d, J=7.5 Hz); 6.81 (1H, s,); 4.70 (2H, s,); 4.26 (2H, t, J=5.2 Hz); 3.61 (7H, m,); 3.14 (3H, s,); 2.38 (2H, m,); 2.36 (3H, s,); 2.25 (3H, m,); 1.91 (3H, s,)

APCI-LC/MS m/z: 479.4 [M+]

### EXAMPLE 234

### tert-butyl 4-[(3-(aminocarbonyl)-4-{[2-ethyl-3-(hydroxymethyl)phenyl]amino}-6-methoxyquinolin-7-yl)oxy]piperidine-1-carboxylate

A mixture of 4-{[2-ethyl-3-(hydroxymethyl)phenyl]amino}-7-hydroxy-6-methoxyquinoline-3-carboxamide (112.6 mg, 0.31 mmole), prepared according to the procedure described in WO 02/092571, tert-butyl 4-[(methylsulfonyl)oxy]piperidine-1-carboxylate (99.7 mg, 0.36 mg) and cesium carbonate (158.5 mg, 0.49 mg) in dimethyl sulfoxide (2 ml) was heated at 70° C. for 10 h. The reaction mixture was cooled and partitioned between ethyl acetate and water. The organic layer was washed with water dried over sodium sulfate, filtrated and concentrated in vacuum. The residue was purified by flash chromatography eluting with dichloromethane/methanol (9.6:0.4) to give the title compound as a yellow powder (39 mg, 23%).

¹H NMR (399.99 MHz, DMSO-d₆) δ 11.06 (1H, s), 8.87 (1H, s), 8.37 (1H, s), 7.69 (1H, s), 7.32 (1H, s), 7.17 (1H, s), 7.05 (1H, s), 6.65 (2H, s), 6.62 (2H, d), 5.16 (1H, s), 4.59 (2H, d), 3.69 (2H, mult), 3.19 (5H, s), 2.79 (2H, d), 2.02 (1H, s), 1.53 (2H, mult), 1.39 (9H, s), 1.19 (3H, t)

APCI-LC/MS m/z: 551.4 [MH+]

### EXAMPLE 235

### tert-butyl 4-({3-(aminocarbonyl)-4-[(2-ethylphenyl)amino]-7-methoxyquinolin-6-yl}oxy)piperidine-1-carboxylate

The title compound was prepared as described in example 235.

APCI-LC/MS m/z: 521.4 [MH+]

### EXAMPLE 236

### 3-(aminocarbonyl)-4-[(2-ethylphenyl)amino]-7-methoxyquinolin-6-yl propane-2-sulfonate

To a solution of 4-[(2-ethylphenyl)amino]-6-hydroxy-7-methoxyquinoline-3-carboxamide trifluoroacetate, prepared according to the procedure described in WO 02/092571, (77.2 mg, 0.17 mmole), triethylamine (0.5 ml, 3.6 mmole) in 1-methyl-2-pyrrolidinone was added propane-2-sulfonyl chloride (0.1 ml, 0.89 mmole). After stirring at room temperature for 48 h, the reaction mixture was partitioned between ethyl acetate and water. The organic layer was washed with water, dried over sodium sulfate and concentrated in vacuum. The residue was purified by preparative HPLC to give the title compound as a white solid (24.6 mg, 32%).

¹H NMR (399.99 MHz, DMSO-d₆) δ 11.14 (1H, s), 9.01 (1H, s), 8.36 (1H, s), 7.70 (1H, s), 7.45 (1H, s), 7.34 (1H, dd), 7.20 (1H, s), 7.14 (2H, t), 7.07 (1H, td), 6.75 (1H, d), 3.94 (3H, s), 3.13 (1H, t), 2.70 (2H, q), 1.17 (6H, d), 1.16 (3H, t)

APCI-LC/MS m/z: 444.1 [MH+]

### EXAMPLE 237

### 4-{[2-ethyl-3-(hydroxymethyl)phenyl]amino}-6-methoxy-7-(piperidin-4-yloxy)quinoline-3-carboxamide

Tert-butyl 4-[(3-(aminocarbonyl)-4-{[2-ethyl-3-(hydroxymethyl)phenyl]amino}-6-methoxyquinolin-7-yl)oxy]piperidine-1-carboxylate (32 mg, 0.06 mmole) described in example 235 was dissolved in dichloromethane (5 ml) cooled on ice and trifluoroacetic acid (5 ml) was added. After 1 h stirring at room temperature the solvent was evaporated. The residue was purified by preparative HPLC to give the title compound as a white powder (7 mg, 26%).

¹H NMR (399.99 MHz, DMSO-d₆) δ11.03 (1H, s), 8.84 (1H, s), 8.25 (1H, s), 7.56 (1H, s), 7.24 (1H, s), 7.16 (1H, d), 7.03 (1H, t), 6.62 (1H, s), 6.59 (1H, d), 4.63 (3H, s), 3.17 (3H, s), 2.93 (2H, q), 2.77 (2H, q), 2.60 (3H, t), 1.95 (2H, dd), 1.44 (2H, mult), 1.18 (3H, t)

APCI-LC/MS m/z: 451.2 [MH+]

### EXAMPLE 238

### 4-[(2-ethylphenyl)amino]-7-methoxy-6-(piperidin-4-yloxy)quinoline-3-carboxamide

The title compound was prepared as described in example 238

¹H NMR (399.99 MHz, DMSO-d₆) δ 10.83 (1H, s), 8.88 (1H, s), 8.29 (1H, br s), 7.62 (1H, br s), 7.32 (1H, dd), 7.25 (1H, s), 7.04 (2H, quintet), 6.67 (1H, s), 6.60 (1H, d), 3.87 (3H, s), 3.54 (1H, mult), 2.77 (4H, mult), 2.20 (2H, d), 1.48 (2H, s), 1.21 (5H, mult)

APCI-LC/MS m/z: 421.2 [MH+]

### EXAMPLE 239

### 6-[3-(cyclopropylamino)-2-hydroxypropoxy]-4-[(2-ethylphenyl)amino]-7-methoxyquinoline-3-carboxamide

To a mixture of 4-[(2-ethylphenyl)amino]-6-hydroxy-7-methoxyquinoline-3-carboxamide, prepared according to the procedure described in WO 02/092571, (0.070 g, 0.2 mmol) and Cs₂CO₃ (0.100 g, 0.3 mmol) in NMP (3.0 ml) epibromohydrine (0.034 g, 0.25 mmol) was added and the mixture was heated at 90° C. for 0.5 h. After cooling cyclopropylamine (0.05 g 0.87 mmol) was added and the mixture heated at 70° C. over night. After cooling the reaction mixture was diluted with water (2.0 ml) and purified with preparative HPLC using a gradient of acetonitrile/water at a flow rate of 20 ml/min.

¹H NMR (399.99 MHz, DMSO-d₆) δ 10.89 (s, 1H), 8.89 (s, 1H), 8.29 (s, 1H), 7.61 (s, 1H), 7.34-7.31 (m, 1H), 7.26 (s, 1H), 7.09-7.06 (m, 2H), 6.68-6.66 (m, 1H), 6.65 (s, 1H), 4.83 (d, 1H), 3.90 (s, 3H), 3.70 (q, 1H), 3.29-3.18 (m, 2H), 2.72 (q, 2H), 2.55-2.45 (m, 2H), 2.05-2.00 (m, 1H), 1.25 (t, 3H), 0.36-0.33 (m, 2H), 0.18-0.14 (m, 2H)

APCI-MS m/z: 451.5 [MH+]

The title compounds of examples 240-247 were prepared in analogous manner to example 239.

### EXAMPLE 240

### 6-{3-[(2-cyanoethyl)amino]-2-hydroxypropoxy}-4-[(2-ethylphenyl)amino]-7-methoxyquinoline-3-carboxamide

APCI LC-MS m/z: 464.1 [MH+]

### EXAMPLE 241

### 4-[(2-ethylphenyl)amino]-6-[2-hydroxy-3-(2-hydroxypyrrolidin-1-yl)propoxy]-7-methoxyquinoline-3-carboxamide

APCI LC-MS m/z: 481.3 [MH+]

### EXAMPLE 242

### 4-[(2-ethylphenyl)amino]-6-(2-hydroxy-3-piperazin-1-ylpropoxy)-7-methoxyquinoline-3-carboxamide

APCI LC-MS m/z: 480.3 [MH+]

### EXAMPLE 243

### 6-{[(2R)-3-(cyclopropylamino)-2-hydroxy-2-methylpropyl]oxy}-4-[(2-ethylphenyl)amino]-7-methoxyquinoline-3-carboxamide

APCI LC-MS m/z: 465.6 [MH+]

### EXAMPLE 244

### 6-{[(2S)-3-(cyclopropylamino)-2-hydroxy-2-methylpropyl]oxy}-4-[(2-ethylphenyl)amino]-7-methoxyquinoline-3-carboxamide

APCI LC-MS m/z: 465.6 [MH+]

### EXAMPLE 245

### 6-[3-(cyclopropylamino)-2-hydroxypropoxy]-4-{[2-ethyl-3-(hydroxymethyl)phenyl]amino}-7-methoxyquinoline-3-carboxamide

APCI LC-MS m/z: 481.2 [MH+]

### EXAMPLE 246

### 6-{[(2R)-3-(cyclopropylamino)-2-hydroxypropyl]oxy}-4-[(2-ethylphenyl)amino]-7-methoxyquinoline-3-carboxamide

APCI LC-MS m/z: 451.5 [MH+]

### EXAMPLE 247

### 6-{[(2S)-3-(cyclopropylamino)-2-hydroxypropyl]oxy}-4-[(2-ethylphenyl)amino]-7-methoxyquinoline-3-carboxamide

APCI LC-MS m/z: 451.5 [MH+]

### EXAMPLE 248

### 3-(aminocarbonyl)-4-[(2-ethylphenyl)amino]-7-methoxyquinolin-6-yl 2-methylpropanoate

The title compound was prepared in an analogues way to example 236.

APCI-MS m/z: 408.4 [MH+]

### EXAMPLE 249

### 6,7-diethoxy-4-[(4-methyl-1-oxo-1,2-dihydroisoquinolin-5-yl)amino]quinoline-3-carboxamide

The title compound was prepared as described in WO 02/092571 starting from 5-amino-4-methylisoquinolin-1(2H)-one and 4-chloro-6,7-diethoxyquinoline-3-carboxamide.

¹H NMR (399.99 MHz, DMSO-d₆) δ 11.28 (1H, s), 11.26 (1H, d), 8.85 (1H, s), 8.27 (1H, s), 8.11 (1H, d), 7.58 (1H, s), 7.33 (1H, t), 7.22 (1H, s), 7.11 (1H, d), 6.99 (1H, d), 6.64 (1H, s), 4.14 (2H, mult), 3.37 (3H, mult), 3.25 (2H, mult), 1.35 (3H, t), 0.92 (3H, t)

APCI-LC/MS m/z: 433.2 [MH+]

### EXAMPLE 250

### 6,7-diethoxy-4-[(4-methyl-1-oxo-1,2,3,4-tetrahydroisoquinolin-5-yl)amino]quinoline-3-carboxamide

The title compound was prepared in an analogues manner as described in WO 02/092571 starting from 5-amino-4-methyl-3,4-dihydroisoquinolin-1(2H)-one and 4-chloro-6,7-diethoxyquinoline-3-carboxamide.

¹H NMR (399.99 MHz, DMSO-d₆) δ 11.28 (1H, s), 11.26 (1H, d), 8.85 (1H, s), 8.27 (1H, s), 8.12 (1H, d), 7.58 (1H, s), 7.33 (1H, t), 7.22 (1H, s), 7.11 (1H, d), 6.99 (1H, d), 6.64 (1H, s), 4.14 (1H, quintetd), 3.37 (3H, dq), 3.23 (4H, mult), 1.35 (3H, t), 0.92 (3H, t)

APCI-LC/MS m/z: 435.3 [MH+]

### EXAMPLE 251

### tert-butyl 5-{[3-(aminocarbonyl)-6,7-diethoxyquinolin-4-yl]amino}-3,4-dihydroisoquinoline-2(1H)-carboxylate

A mixture of 4-chloro-6,7-diethoxyquinoline-3-carboxamide (178 mg, 0.61 mmole, prepared according to WO 02/092571), tert-butyl 5-amino-3,4-dihydroisoquinoline-2(1H)-carboxylate (198 mg, 0.80 mmole), acetic acid (7 μl) in NMP (3 ml) was heated over night at 110 C. The reaction mixture was cooled, partitioned between ethyl acetate and sodium hydrogen carbonate solution.

The organic layer was washed with water, dried over sodium sulfate and concentrated in vacuum. The residue was purified by flash chromatography eluting with dichloromethane/methanol (10:0.5) to give the title compound as a light brown powder (214 mg, 69%).

¹H NMR (399.99 MHz, DMSO-d₆) δ 10.63 (1H, s), 8.84 (1H, s), 8.24 (1H, br s), 7.58 (1H, br s), 7.22 (1H, s), 7.06 (1H, t), 6.95 (1H, d), 6.65 (2H, s), 6.61 (2H, d), 4.53 (2H, s), 4.15 (2H, q), 3.59 (2H, t), 3.49 (2H, d), 2.70 (2H, t), 1.39 (9H, s), 1.36 (3H, t), 1.06 (3H, t).

APCI-LC/MS m/z: 507.2 [MH+]

### EXAMPLE 252

### 6,7-diethoxy-4-(1,2,3,4-tetrahydroisoquinolin-5-ylamino)quinoline-3-carboxamide

The title compound was prepared in a similar way described in example 117

¹H NMR (399.99 MHz, DMSO-d₆) δ 10.62 (1H, s), 8.84 (1H, s), 8.24 (1H, s), 7.58 (1H, s), 7.22 (1H, s), 6.96 (1H, d), 6.80 (1H, d), 6.70 (1H, s), 6.52 (1H, d), 4.15 (2H, d), 3.85 (2H, s), 3.51 (2H, s), 2.98 (2H, s), 2.61 (2H, s), 1.37 (3H, s), 1.15 (3H, s)

APCI-LC/MS m/z: 407.2 [MH+]

### EXAMPLE 253

### 4-{[3-(azidomethyl)-2-ethylphenyl]amino}-6-[3-(cyclopropylamino)propoxy]-7-methoxyquinoline-3-carboxamide

The title compound was prepared analogous manner to example 1 using sodium acid.

APCI-LC/MS m/z: 490.3 [MH+]

### EXAMPLE 254

### 4-{[3-(aminomethyl)-2-ethylphenyl]amino}-6-[3-(cyclopropylamino)propoxy]-7-methoxyquinoline-3-carboxamide

The title compound was prepared according to the procedure described in WO 02/092571 using 5% Palladium-charcoal and the compound described in example 253.

APCI-LC/MS m/z: 464.3 [MH+]

### EXAMPLE 255

### 4-{[3-(aminomethyl)-2-ethylphenyl]amino}-7-{3-[isobutyryl(isopropyl)amino]propoxy}-6-methoxyquinoline-3-carboxamide

The title compound was prepared in an analogous manner to example 255 using the compound described in example 185.

APCI-LC/MS m/z: 536.4 [MH+]

### EXAMPLE 256

### 4-{[3-(azidomethyl)-2-ethylphenyl]amino}-6-[3-(cyclopropylamino)-2-hydroxypropoxy]-7-methoxyquinoline-3-carboxamide

The title compound was prepared in an analogous manner to example 240

APCI-LC/MS m/z: 506.6 [MH+]

### EXAMPLE 257

### 4-{[3-(aminomethyl)-2-ethylphenyl]amino}-6-[3-(cyclopropylamino)-2-hydroxypropoxy]-7-methoxyquinoline-3-carboxamide

The title compound was prepared in an analogous manner to example 254

APCI-LC/MS m/z: 480.6 [MH+]

### EXAMPLE 258

### 4-({3-[(acetylamino)methyl]-2-ethylphenyl}amino)-6-{3-[acetyl(cyclopropyl)amino]-2-hydroxypropoxy}-7-methoxyquinoline-3-carboxamide

The title compound was prepared in an analogous manner to example 119 using compound 257 and acetic acid anhydride.

APCI-LC/MS m/z: 564.6 [MH+]

The title compounds of examples 259-261 were prepared in analogous manner to example 239.

### EXAMPLE 259

### 6-[3-(cyclopropylamino)-2-hydroxypropoxy]-4-{[2-ethyl-3-(1H-imidazol-1-ylmethyl)phenyl]amino}-7-methoxyquinoline-3-carboxamide

APCI-LC/MS m/z: 480.6 [MH+]

### EXAMPLE 260

### 6-[3-(cyclopropylamino)-2-hydroxypropoxy]-4-{[2-ethyl-3-(1H-pyrazol-1-ylmethyl)phenyl]amino}-7-methoxyquinoline-3-carboxamide

The title compound was prepared from 4-{[2-ethyl-3-(1H-imidazol-1-ylmethyl)phenyl]amino}-6-hydroxy-7-methoxyquinoline-3-carboxamide, epibromohydrine and cyclopropylamine as described in example 239.

¹H NMR (400 MHz, DMSO-d₆) δ 11.00 (1H, s,); 8.90 (1H, s,); 8.32 (1H, s,); 7.72 (1H, s,); 7.64 (1H, s,); 7.26 (1H, s,); 7.11 (1H, s,); 7.05 (2H, t, J=7.8 Hz); 6.94 (1H, s,); 6.77 (1H, d, J=7.6 Hz); 6.62 (2H, mult,); 5.34 (2H, s,); 4.89 (1H, d, J=4.3 Hz); 3.90 (3H, s,); 3.72 (1H, s,); 2.83 (2H, q, J=7.2 Hz); 2.06 (1H, mult,); 0.35 (2H, mult,); 0.16 (1H, d, J=1.8 Hz)

APCI-LC/MS m/z: 531.6 [MH+]

### EXAMPLE 261

### 6-{[(2S)-3-(cyclopropylamino)-2-hydroxypropyl]oxy}-4-{[2-ethyl-3-(morpholin-4-ylmethyl)phenyl]amino}-7-methoxyquinoline-3-carboxamide

APCI-LC/MS m/z: 550.7 [MH+]

### EXAMPLE 262

### amino {6,7-diethoxy-4-[(2-ethylphenyl)amino]quinolin-3-yl}methanol

Red-A1 (5.3 mg, 0.13 mmol) was added slowly to a mixture of 6,7-diethoxy-4-[(2-ethylphenyl)amino]quinoline-3-carboxamide (10 mg, 0.26 mmol) prepared according to the procedure described in WO 02/092571 in THF under argon and stirred at 50° C. for 18 hrs. The resulting mixture was washed with water and the organic layers dried over natriumsulfate, filtered and concentrated. The resulting crude product was purified on HPLC to give 1 mg (2.62 mmol, 10%) of the desired product.

APCI-MS: m/z 382.5 [MH+]

### EXAMPLE 263

### 6-[3-(cyclopropylamino)propoxy]-4-{[2-ethyl-3-(1H-imidazol-1-ylmethyl)phenyl]amino}-7-methoxyquinoline-3-carboxamide

The title compound was prepared in an analogous manner to example 253

APCI-LC/MS m/z: 515.4 [MH+]

### EXAMPLE 264

### 4-{[2-ethyl-3-(1H-imidazol-1-ylmethyl)phenyl]amino}-6-methoxy-7-(2-methoxyethoxy)quinoline-3-carboxamide

### a) 4-{[2-ethyl-3-(hydroxymethyl)phenyl]amino}-6-methoxy-7-(2-methoxyethoxy)quinoline-3-carboxamide

A mixture of 4-{[2-ethyl-3-(hydroxymethyl)phenyl]amino}-7-hydroxy-6-methoxyquinoline-3-carboxamide,) prepared according to the procedure described in WO 02/092571, (32.2 mg, 0.09 mmol), 2-bromoethyl methyl ether (23.7 mg, 0.17 mmol), cesium carbonate (45.3 mg, 0.14 mmol) and NMP (1 ml) was heated at 60° C. for 4 h. After cooling the reaction mixture was diluted with water and purified by preparative HPLC to give 19 mg of the compound

APCI-LC/MS m/z: 426.3 [MH+].

### b) The Title Compound was then Prepared in an Analogous Manner to Example 1

¹H NMR (399.99 MHz, DMSO-d₆): δ 11.02 (1H, s); 8.89 (1H, d, J=5.2 Hz); 8.31 (1H, br s); 7.72 (1H, s); 7.64 (1H, br s); 7.26 (1H, s); 7.09 (1H, s); 7.06 (1H, d, J=7.7 Hz); 6.93 (1H, s); 6.87 (2H, d, J=7.5 Hz); 6.64 (1H, d, J=7.7 Hz); 6.57 (1H, s); 5.33 (2H, s); 4.22 (2H, t, J=4.3 Hz); 3.70 (2H, t, J=4.4 Hz); 3.31 (2H, br s); 3.18 (3H, br s); 2.86 (2H, q, J=7.4 Hz); 1.03 (3H, t, J=7.4 Hz).

APCI-LC/MS m/z: 476.4 [MH+]

### EXAMPLE 265

### 6-(ethylamino)-4-{[2-ethyl-3-(1H-imidazol-1-ylmethyl)phenyl]amino}-7-methoxyquinoline-3-carboxamide

### a) ethyl 6-(ethylamino)-4-{[2-ethyl-3-(1H-imidazol-1-ylmethyl)phenyl]amino}-7-methoxyquinoline-3-carboxylate

In a schlenk were placed ethyl 6-bromo-4-{[2-ethyl-3-(1H-imidazol-1-ylmethyl)phenyl]amino}-7-methoxyquinoline-3-carboxylate (71.2 mg, 0.14 mmol) prepared in an analogues way to example 1, tris(dibenzylideneacetone) dipalladium(0) (12.1 mg, 0.01 mmol) bis(diphenylphospino)1.1naphtalene (28.3 mg, 0.05 mmol), cesium carbonate (83 mg, 0.26 mmol), ethyl amine (0.34 mg, 7.6 mmol) and toluene (10 ml) under argon. The vessel was sealed and heated at 75° C. for 48 h. The reaction mixture was cooled and partitioned between ethyl acetate and water. The organic layer was dried over sodium sulphate, filtrated and concentrated in vacuum. The residue was purified by preparative HPLC to give 24 mg of the desired product.

APCI-LC/MS m/z: 474.3 [MH+]

b) Potassium cyanide (5 mg) and the product from the first step were suspended in dry methanol (10 ml) saturated with ammonia. The schlenk was sealed and heated at 65° C. for 50 h. The reaction mixture was cooled and concentrated in vacuum. The residue was purified by preparative HPLC to give the title compound as a white solid (8 mg, 13%).

¹H NMR (399.99 MHz, DMSO-d₆): δ 10.65 (1H, s); 8.73 (1H, s); 8.23 (1H, br s); 7.68 (1H, s); 7.56 (1H, br s); 7.12 (1H, s); 7.06 (1H, s); 6.98 (2H, t, J=7.8 Hz); 6.92 (1H, s); 6.81 (1H, d, J=7.5 Hz); 6.47 (1H, d, J=7.8 Hz); 6.05 (1H, s); 5.29 (3H, m); 3.91 (3H, s); 2.84 (2H, d, J=7.2 Hz); 2.52 (1H, br s); 1.02 (3H, t, J=7.4 Hz); 0.75 (3H, t, J=7.1 Hz).

APCI-LC/MS m/z: 445.3 [MH+].

The title compounds of examples 266-268 were prepared in analogous manner to example 265.

### EXAMPLE 266

### 6-[(2,2-dimethoxyethyl)amino]-4-[(2-ethylphenyl)amino]-7-methoxyquinoline-3-carboxamide

¹H NMR (399.99 MHz, DMSO-d₆): δ 10.61 (1H, s); 8.75 (1H, s); 8.24 (1H, s); 7.57 (1H, s); 7.28 (1H, m); 7.16 (1H, s); 7.00 (2H, m); 6.50 (1H, m); 6.24 (1H, s); 5.24 (1H, t, J=5.8 Hz); 4.15 (1H, t, J=5.5 Hz); 3.94 (3H, s); 3.12 (6H, s); 2.73 (2H, q, J=7.5 Hz); 2.65 (2H, t, J=5.7 Hz); 1.26 (3H, t, J=7.5 Hz).

APCI-LC/MS m/z: 425.4 [MH+]

### EXAMPLE 267

### 6-[(3,3-diethoxypropyl)amino]-4-[(2-ethylphenyl)amino]-7-methoxyquinoline-3-carboxamide

¹H NMR (299.946 MHz, DMSO-d₆) δ 10.58 (1H, s), 8.73 (1H, s), 8.28 (1H, br s), 7.62 (1H, br s), 7.24 (1H, mult), 7.13 (1H, s), 6.98 (2H, dquintet), 6.51 (1H, mult), 6.14 (1H, s), 5.42 (1H, t), 4.32 (1H, t), 3.91 (3H, s), 3.49 (2H, mult), 3.36 (2H, mult), 2.72 (2H, q), 2.53 (2H, q), 1.51 (2H, dd), 1.25 (3H, t), 1.08 (6H, t)

APCI-LC/MS m/z: 467.4 [MH+]

### EXAMPLE 268

### tert-butyl [2-({3-(aminocarbonyl)-4-[(2-ethylphenyl)amino]-7-methoxyquinolin-6-yl}amino)ethyl]carbamate

APCI-LC/MS m/z: 480.3 [MH+]

### EXAMPLE 269

### tert-butyl {2-[(3-(aminocarbonyl)-4-{[2-ethyl-3-(hydroxymethyl)phenyl]amino}-7-methoxyquinolin-6-yl)amino]ethyl}carbamate

### a) Ethyl 6-bromo-4-{[3-({[tert-butyl(dimethyl)silyl]oxy}methyl)-2-ethylphenyl]amino}-7-methoxyquinoline-3-carboxylate

A mixture of ethyl 6-bromo-4-{[2-ethyl-3-(hydroxymethyl)phenyl]amino}-7-methoxyquinoline-3-carboxylate (406 mg, 0.89 mmol), tert-butyl(chloro) dimethylsilane (0.95 g, 6.3 mmol), imidazole (1.9 g, 27.9 mmol) in DMF (3 ml) was stirred under argon at room temperature for 48 hours. The reaction mixture was then partitioned between ethyl acetate and water. The organic layer was washed with water, dried over sodium sulphate, filtrated and concentrated in vacuum. The residue was purified by flash chromatography eluting with dichloromethane/methanol (97:3) to give the title compound as grey powder (309 mg, 60%).

¹H NMR (399.99 MHz, DMSO-d₆): δ 10.24 (1H, s); 9.01 (1H, s); 7.54 (1H, s); 7.37 (1H, s); 7.29 (1H, d, J=7.4 Hz); 7.11 (1H, t, J=7.8 Hz); 6.81 (1H, d, J=7.8 Hz); 4.79 (2H, s); 4.31 (2H, q, J=7.1 Hz); 3.94 (3H, s); 2.76 (2H, q, J=7.5 Hz); 1.32 (3H, t, J=7.1 Hz); 1.15 (3H, t, J=7.5 Hz); 0.89 (9H, s, J=2.9 Hz); 0.09 (6H, s, J=3.1 Hz).

APCI-LC/MS m/z: 573.1, 574.2, 575.1 [M+], [M+1], [M+2]

### b) Ethyl 6-({2-[(tert-butoxycarbonyl)amino]ethyl}amino)-4-{[3-({[tert-butyl(dimethyl)silyl]oxy}methyl)-2-ethylphenyl]amino}-7-methoxyquinoline-3-carboxylate

A mixture of ethyl 6-bromo-4-{[3-({[tert-butyl(dimethyl)silyl]oxy}methyl)-2-ethylphenyl]amino}-7-methoxyquinoline-3-carboxylate (250 mg, 0.44 mmol) tris(dibenzylideneacetone) dipalladium(0) (21 mg, 0.02 mmol) bis(diphenylphospino)1.1naphtalene (48 mg, 0.08 mmol), cesium carbonate (230 mg, 0.71 mmol), N-(2-Aminoethyl)carbamic Acid ter-tbutylester (101 mg, 0.63 mmol) and toluene (8 ml) was placed in a schlenk under argon. The Schlenk vessel was sealed and the reaction mixture was heated at 85° C. over night. After cooling the reaction mixture was partitioned between ethyl acetate and water. The organic layer was washed with water dried over sodium sulphate, filtrated and concentrated in vacuum. The residue was purified by flash chromatography eluting with dichloromethane/methanol (100:3) to give the title compound as a yellow powder (205 mg, 73%).

¹H NMR (399.988 MHz, CDCl₃): δ 10.21 (1H, s); 9.03 (1H, s); 7.25 (1H, d, J=7.3 Hz); 7.21 (2H, s); 7.06 (1H, t, J=7.8 Hz); 6.76 (1H, d, J=7.8 Hz); 4.83 (2H, s); 4.64 (1H, br s); 4.54 (1H, br s); 4.43 (2H, q, J=7.1 Hz); 3.97 (3H, s); 3.00-2.83 (5H, m); 2.83-2.61 (4H, m); 1.48-1.43 (13H, m); 1.31 (3H, t, J=7.5 Hz); 0.98 (9H, s, J=2.8 Hz); 0.16 (6H, s, J=3.0 Hz);

APCI-LC/MS m/z: 653.3 [MH+]

### c) tert-butyl {2-[(3-(aminocarbonyl)-4-{[2-ethyl-3-(hydroxymethyl)phenyl]amino}-7-methoxyquinolin-6-yl)amino]ethyl}carbamate

In a high pressure flask were placed ethyl 6-({2-[(tert-butoxycarbonyl)amino]ethyl}amino)-4-{[3-({[tert-butyl(dimethyl)silyl]oxy}methyl)-2-ethylphenyl]amino}-7-methoxyquinoline-3-carboxylate (148 mg, 0.23 mmol), potassium cyanide (7 mg, 0.1 mmol) and dry methanol (10 ml) saturated with ammonia. The flask was sealed and heated at 55° C. for 96 hours. After cooling the reaction mixture was concentrated in vacuum and the residue was stirred with tetrabutylammonium fluoride hydrate (150 mg, 0.57 mmol) in tetrahydrofuran (5 ml) for 1 hour. The reaction mixture was partitioned between ethyl acetate and water. The organic layer was washed with water, dried over sodium sulphate, filtered and concentrated in vacuum. The residue was purified by preparative HPLC to give the title compound as a light yellow powder (52 mg, 44%).

The title compound was prepared in an analogous manner to example 266 using ethyl 6-bromo-4-[(2-ethylphenyl)amino]-7-methoxyquinoline-3-carboxylate and (3-Amino-propyl)-cyclopropyl-carbamic acid tert-butyl ester. Removal of the carbamaic acid tert-butyl ester derivative was performed with TFA according to example 117.

¹H NMR (399.99 MHz, DMSO-d₆): δ 10.69 (1H, s); 8.73 (1H, s); 8.21 (1H, br s); 7.53 (1H, br s); 7.16-7.07 (2H, m); 6.96 (1H, t, J=7.7 Hz); 6.70 (1H, t, J=5.3 Hz); 6.45 (1H, d, J=7.8 Hz); 6.10 (1H, s); 5.37 (1H, t, J=4.8 Hz); 5.12 (1H, t, J=5.3 Hz); 4.59 (2H, d, J=5.3 Hz); 3.90 (3H, s); 2.81 (4H, m); 1.36 (9H, s); 1.27 (2H, s); 1.21 (3H, t, J=7.4 Hz);

APCI-LC/MS m/z: 510.3 [MH+]

### EXAMPLE 270

### 6-{[3-(cyclopropylamino)propyl]amino}-4-[(2-ethylphenyl)amino]-7-methoxyquinoline-3-carboxamide

### a) Ethyl 6-bromo-4-[(2-ethylphenyl)amino]-7-methoxyquinoline-3-carboxylate was prepared according to WO 02/092571

¹H NMR (399.99 MHz, DMSO-d₆): δ 10.12 (1H, s); 9.01 (1H, s); 7.67 (1H, s); 7.44-7.38 (2H, m); 7.25 (1H, t, J=7.3 Hz); 7.16 (1H, t, J=12.5 Hz); 6.93 (1H, d, J=7.7 Hz); 4.27 (2H, q, J=7.1 Hz); 3.98 (3H, s); 2.71 (2H, q, J=7.5 Hz); 1.32 (3H, t, J=7.1 Hz); 1.21 (3H, t, J=7.5 Hz).

APCI-LC/MS m/z: 429.1, 431.1 [MH+]

### b) (3-Amino-propyl)-cyclopropyl-carbamic acid tert-butyl ester

### tert-butyl cyclopropyl(3-hydroxypropyl)carbamate

A mixture of 3-bromopropan-1-ol (4.5 g, 32.4 mmol), cyclopropylamine (12.4 g, 216.5 mmol) and tetrahydrofuranne (40 ml) was heated at 60° C. for 7 h. The reaction was cooled to room temperature, concentrated in vacuum, diluted with a mixture of tetrahydrofuranne (20 ml)/triethylamine (10 ml) and again concentrated in vacuum.

To the residue was added di-tert-butyl dicarbonate (7.2 g, 33.0 mmol), tetrahydrofuranne (35 ml) and triethylamine (5 ml). The suspension was heated at 50° C. over night then cooled to room temperature, diluted with ether, filtered and the filtrate was concentrated in vacuum. The residue was purified by flash chromatography eluting with dichloromethane/methanol (100:3) to give the title compound as colourless oil (3.1 g, 44%).

¹H NMR (299.944 MHz, CDCl₃): δ 3.56 (2H, quintet, J=5.7 Hz); 3.38 (2H, t, J=6.1 Hz); 2.45 (1H, ddd, J=10.8 7.0 3.9 Hz); 1.70 (2H, quintet, J=8.1 Hz); 1.46 (9H, s); 0.77-0.68 (2H, m); 0.62-0.56 (2H, m).

### tert-butyl 3-bromopropyl(cyclopropyl)carbamate

To an is cooled solution of tert-butyl cyclopropyl(3-hydroxypropyl)carbamate (1.6 g, 7.4 mmol), triphenylphosphine (2.5 g, 9.7 mmol) and tetrahydrofuran (25 ml) was added carbon tetrabromide (3.2 g, 9.7 mmol) under 20 minutes. The mixture was stirred for 30 minutes at 0° C. and then allowed to reach room temperature. After 3 hours at ambient temperature the reaction mixture was diluted with diethyl ether and the precipitate was removed by filtration. The filtrate was concentrated in vacuum and the residue was purified by flash chromatography eluting with dichloromethane/heptane (2:1) to give the title compound as colourless oil (1.2 g, 59%).

¹H NMR (399.988 MHz, CDCl₃): δ 3.39 (2H, t, J=6.7 Hz); 3.33 (2H, t, J=7.1 Hz); 2.49 (1H, septet, J=5.1 Hz); 2.11 (2H, quintet, J=6.9 Hz); 1.45 (9H, s); 0.75 (2H, td, J=7.1 5.1 Hz); 0.60 (2H, m).

### tert-butyl 3-azidopropyl(cyclopropyl)carbamate

A mixture of tert-butyl 3-bromopropyl(cyclopropyl)carbamate (1.1 g, 3.9 mmol), sodium azide (0.33 g, 5.1 mmol) and 1-methyl-2-pyrrolidinone (7 ml) was stirred at ambient temperature over night. The reaction mixture was partitioned between ethyl acetate and water. The organic layer was washed with water dried over sodium sulphate, filtrated and concentrated in vacuum. The residue was purified by flash chromatography eluting with dichloromethane to give the title compound as colourless oil (0.92 g, 96%).

¹H NMR (399.988 MHz, CDCl₃): δ 3.29 (4H, q, J=13.1 Hz); 2.48 (1H, septet, J=5.7 Hz); 1.82 (2H, quintet, J=7.0 Hz); 1.45 (9H, s); 0.74 (2H, m); 0.59 (2H, m).

### (3-Amino-propyl)-cyclopropyl-carbamic acid tert-butyl ester

A mixture of tert-butyl 3-azidopropyl(cyclopropyl)carbamate (0.9 g, 3.7 mmol), 5% palladium on carbon (60 mg) in ethanol (15 ml) and ethyl acetate (15 ml) was stirred vigorously under 1 atmosphere of hydrogen for 19 h. The hydrogen atmosphere was changed twice under the period of reaction time. The catalyst was filtered off and the filtrate was concentrated to give the title compound as colourless oil (0.79 g, 98%).

¹H NMR (399.988 MHz, CDCl₃): δ 3.27 (2H, t, J=6.9 Hz); 2.70 (2H, t, J=6.8 Hz); 2.45 (1H, dt, J=6.9 3.4 Hz); 1.76 (2H, s); 1.69 (2H, quintet, J=6.9 Hz); 1.44 (9H, s); 0.72 (2H, dd, J=12.2 6.9 Hz); 0.57 (2H, m).

### c) 6-{[3-(cyclopropylamino)propyl]amino}-4-[(2-ethylphenyl)amino]-7-methoxyquinoline-3-carboxamide

The title compound was prepared in an analogous manner to example 266 using ethyl 6-bromo-4-[(2-ethylphenyl)amino]-7-methoxyquinoline-3-carboxylate and (3-Amino-propyl)-cyclopropyl-carbamic acid tert-butyl ester. Removal of the carbamaic acid tert-butyl ester derivative was performed with TFA according to example 117.

¹H NMR (399.99 MHz, DMSO-d₆): δ 10.57 (1H, s); 8.73 (1H, s); 8.22 (1H, br s); 7.55 (1H, br s); 7.27 (1H, m); 7.13 (1H, s); 6.99 (2H, m); 6.50 (1H, m); 6.14 (1H, s); 5.65 (1H, t, J=5.5 Hz); 3.92 (3H, s); 2.74 (2H, q, J=7.4 Hz); 2.58 (2H, q, J=6.4 Hz); 2.40 (2H, t, J=6.2 Hz); 2.00-1.89 (2H, m); 1.35-1.22 (5H, m); 0.33 (2H, m); 0.17 (2H, m).

APCI-LC/MS m/z: 434.5 [MH+]

The title compounds of Examples 271-274 were prepared in analogous manner to the methods described above.

### EXAMPLE 271

### 4-(2,3-dihydro-1H-inden-1-ylamino)-6,7-dimethoxyquinoline-3-carboxamide

APCI LC-MS m/z: 364.1 [MH+]

### EXAMPLE 272

### 6,7-diethoxy-4-[(2-methylcyclohexyl)amino]quinoline-3-carboxamide

APCI LC-MS m/z: 372.3 [MH+]

### EXAMPLE 273

### 4-{[(3S)-1-(cyanoacetyl)pyrrolidin-3-yl]amino}-6,7-dimethoxyquinoline-3-carboxamide

APCI LC-MS m/z: 384.1 [MH+]

### EXAMPLE 274

### 4-{[(3S)-1-(cyanoacetyl)piperidin-3-yl]amino}-6,7-dimethoxyquinoline-3-carboxamide

APCI LC-MS m/z: 398.1 [MH+]

### Pharmacological Data

### JAK3 HTRF Assay

The JAK3 kinase assay utilizes a fusion protein (Jak3 kinase domain fused to Glutathione S-transferase, GST) coexpressed in E. Coli with GroEL/S, and purified by affinity chromatography on Glutathione Sepharose. The enzyme is diluted in 10 mM Tris-HCl, 150 mM NaCl, 5% mannitol, 2 mM 2-mercaptoethanol and 30% glycerol. The substrate in the kinase reaction is a biotinylated peptide of the autophosphorylation site of JAK3 (biotin-LPDKDYYVVREPG) used at 2 μM. Assay conditions are as follows: JAK3, compound and substrate are incubated in 25 mM Trizma base, 5 mM MgCl₂, 5 mM MnCl₂, 0.05% TritonX-100 and 2 μM ATP for 45 min at RT. Reaction volume is 20 μM. Stopsolution is added for a final concentration of 100 μM EDTA. Finally 0.065 mg/ml PT66-K and 10.42 μM SA-XL665 are added in 50 mM Hepes, 0.5 M KF and 0.1% BSA. The plate is read in a Discovery instrument after 60 min incubation.

The compounds of the examples have an IC50 less than 10 μM

1. A compound of formula (I) 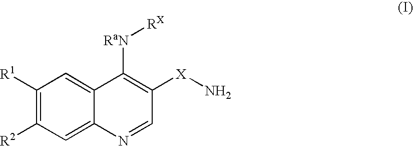
or a pharmaceutically acceptable salt or solvate thereof, wherein
X is —CHOH or —C═O;
R¹ and R², which may be the same or different, represent nitro, cyano, C₁-C₈ alkyl C₁-C₈ alkoxy, hydroxy, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)ₚR⁶, Y(CR³₂)ₚOCOR⁶ or R¹ and R² are linked together as —OCH₂O— or —OCH₂CH₂O—;
R³ groups are independently hydrogen, C₁-C₈ alkyl, hydroxy, C₁-C₈alkoxy or halogen;
p is 0, 1, 2, 3, 4 or 5;
Y is oxygen, CH_{2′}—OSO₂— or NR⁷
R⁴ and R⁵ each independently represent hydrogen or a group selected from C₁-C₈ alkyl, —C₁-C₈ alkoxy, —CO—(C₁-C₈)alkyl, —CO—(C₁-C₈)cycloalkyl, —SO₂—(C₁-C₈)alkyl, —CO—(C₁-C₈)alkoxy, —CO—NR⁷(C₁-C₈)alkyl, C₃-C₈ cycloalkyl, each of which groups may optionally be substituted by one or more hydroxy, cyano, —CONH₂ or —CO—(C₁-C₈)alkoxy groups, or R⁴ and R⁵ together with the nitrogen atom to which they are attached form a 4- to 7-membered, saturated or aromatic heterocyclic ring system optionally containing one or more additional heteroatoms selected from oxygen, sulphur or nitrogen, the ring itself being optionally substituted by at least one substituent selected from hydroxy, C₁-C₈ alkyl, ═O, C₁-C₈ alkoxy or (C₁-C₈ alkoxy)-CO—, or one of R⁴ and R⁵ is hydrogen or C₁-C₈ alkyl and the other is a 5- or 6-membered heterocyclic ring system optionally containing a further oxygen, sulphur or nitrogen atom;
R⁶ is hydrogen, C₁-C₈ alkyl (itself optionally substituted by one or more hydroxy, cyano, halogen or amino groups), phenyl, benzyl, —CO(C₁-C₈)alkyl or a saturated monocyclic 4- to 7-membered ring, which ring may optionally comprise one or more heteroatoms selected from nitrogen, oxygen and sulphur, the ring itself being optionally substituted by at least one substituent selected from C₁-C₈ alkyl, C₁-C₈ alkoxy, ═O, C₁-C₈ alkyl —CO—, or (C₁-C₈ alkoxy)-CO— where any C₁-C₈ alkyl is optionally substituted by one or more hydroxy, cyano, halogen or amino groups;
R⁷ is hydrogen or C₁-C₈ alkyl;
R^{a} is hydrogen or C₁-C₈ alkyl;
R^{x} is a group selected from C₁-C₈ alkyl, C₃-C₈ cycloalkyl or a saturated monocyclic 4- to 7-membered ring comprising one or more heteroatoms selected from nitrogen, oxygen and sulphur, wherein any C₃-C₈ cycloalkyl group or saturated monocyclic 4- to 7-membered ring is optionally substituted by one or more groups selected from hydroxy, azido, cyano, amino, halogen, —CONH₂—, C₁-C₈ alkyl, (C₁-C₈ alkyl)CO—, C₁-C₈ alkoxy, or (C₁-C₈ alkoxy)-CO—, and any C₁-C₈ alkyl, C₁-C₈ alkyl)CO—, C₁-C₈ alkoxy, or (C₁-C₈ alkoxy)-CO— group is itself optionally substituted by one or more substituents selected from hydroxy, azido, cyano, amino, or halogen; or R^{x} is a group Ar;
Ar is selected from phenyl, tetrahydronaphthenyl, indolyl, pyrazolyl, dihydroindenyl, 1-oxo-2,3-dihydroindenyl, indazolyl, dihydroisoquinolyl, oxodihydroisoquinolyl, tetrahydroisoquinolyl or oxotetrahydroisoquinolyl, each of which can be optionally substituted by one or more groups, which may be the same or different, selected from CO₂R⁸, CONR⁹R¹⁰, C₁-C₈ alkyl-NR⁸—C₁-C₈ alkyl, C₁-C₈ alkyl-CONR³—C₁-C₈ alkyl, C₁-C₈ alkyl-CONR⁹R¹⁰, NR³COC₁-C₈ alkyl, C₁-C₈ alkyl substituted by one or more hydroxy, azido or cyano groups or fluorine atoms), C₁-C₈ alkyl-NR¹¹R¹², C₁-C₈ alkyl-OR¹², C₁-C₈ alkyl-SR¹²;
R⁸ is hydrogen or C₁-C₈ alkyl;
R⁹ and R¹⁰ are each independently hydrogen or C₁-C₈ alkyl
R¹¹ is hydrogen or C₁-C₈ alkyl;
R¹² is hydrogen or a group selected from C₁-C₈ alkyl, —(CR¹³₂)ₙR¹⁴, —CO—(CR¹³₂)ₙR¹⁴, —SO₂—(CR¹³ ₂)ₙR¹⁴;
n is between 0 and 5;
R¹³ groups are independently hydrogen, C₁-C₈ alkyl hydroxy, C₁-C₈ alkoxy, hydroxy(C₁-C₈)alkyl, amino or halogen;
R¹⁴ is hydrogen or a group selected from —NR¹⁵R¹⁶, C₁-C₈ alkyl C₂-C₄ alkenyl, C₂-C₄ alkynyl, —COOH, —S(C₁-C₈ alkyl), —SO(C₁-C₈ alkyl), —CONR¹⁵R¹⁶, —CO(C₁-C₈ alkyl), —CO—O—(C₁-C₈ alkyl), or a saturated or unsaturated 4- to 10-membered ring, which ring may optionally comprise one or more heteroatoms selected from nitrogen, oxygen and sulphur, each of which groups may be optionally substituted by one or more hydroxy, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, hydroxy(C₁-C₈)alkyl, C₁-C₈ alkyl, nitro, —CONH₂ groups), C₁-C₈ alkoxy, C₁-C₈ hydroxyalkyl, —C═O, cyano, amino, nitro, halogen, C₁-C₈ alkylsulphonyl or aminosulphonyl groups or by a saturated monocyclic 4- to 7-membered ring, which ring may optionally comprise one or more heteroatoms selected from nitrogen, oxygen and sulphur;
or R¹¹ and R¹², together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—; and
R¹⁵ and R¹⁶, which may be the same or different, represent hydrogen, C₁-C₈ alkyl, —CONH₂ or —C(NH₂)═NH;
provided that when R^{x} is Ar, X is —CO and R¹ and R² are independently nitro, cyano, C₁-C₈ alkyl, C₁-C₈ alkoxy, hydroxyl, aryl, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)ₚR⁶—CH₂(CH₂)ₚOCOR⁶ or R¹ and R² are linked together as —OCH₂O— or —OCH₂CH₂O—, where each R³ group is independently hydrogen, C₁-C₈ alkyl, hydroxy, or halogen, R⁴ and R⁵ each independently represent hydrogen or C₁-C₈ alkyl or R⁴ and R⁵ together with the nitrogen atom to which they are attached form an unsubstituted 4- to 7-membered saturated or aromatic heterocyclic ring system optionally containing a further oxygen, sulphur or NR⁶ group or one of R⁴ and R⁵ is hydrogen or C₁-C₈ alkyl and the other is a 5- or 6-membered heterocyclic ring system optionally containing a further oxygen, sulphur or nitrogen atom;
and R⁶ is selected from hydrogen, (C₁-C₈)alkyl, —CO(C₁-C₈)alkyl, hydroxy substituted (C₁-C₈)alkyl, halogen substituted (C₁-C₈)alkyl, phenyl or benzyl,
then Ar is selected from dihydroisoquinolyl, oxodihydroisoquinolyl, tetrahydroisoquinolyl or oxotetrahydroisoquinolyl, each of which may be optionally substituted,
or Ar is phenyl substituted by at least one substituent selected from azido substituted C₁-C₈ alkyl, C₁-C₈alkyl-NR¹¹R¹²C₁-C₈alkyl-OR¹² or C₁-C₈alkyl-SR¹², wherein R¹² is selected from —(CR¹³₂)ₙR¹⁴—CO—(CR¹³₂)ₙR¹⁴, —SO₂—(CR¹³₂)ₙR¹⁴ or R¹¹ and R¹², together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, (C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—,
provided that Ar is not phenyl substituted by one or more groups selected from C₁-C₈ alkyl-NR¹¹—C₁-C₈ alkyl, C₁-C₈ alkyl-O—C₁-C₈ alkyl or C₁-C₆ alkanoyloxy C₁-C₆ alkyl.

2. A compound of claim 1, wherein
at least one R³ is C₁-C₈ alkoxy, or one of R⁴ and R⁵ is selected from optionally substituted —CO—(C₁-C₈)alkyl, —CO—(C₁-C₈)cycloalkyl, —SO₂—(C₁-C₈)alkyl, —CO—(C₁-C₈)alkoxy, —CO—NR⁷(C₁-C₈)alkyl or C₃-C₈ cycloalkyl, or R⁴ and R⁵ together with the nitrogen atom to which they are attached form a substituted 4- to 7-membered saturated or aromatic heterocyclic ring system optionally containing a further oxygen, sulphur or NR⁶ group,
or R⁶ is selected from —CO(C₁-C₈)alkyl or an optionally substituted saturated monocyclic 4- to 7-membered ring, which ring may optionally comprise one or more heteroatoms selected from nitrogen, oxygen and sulphur, and which may be optionally substituted by at least one substituent selected from C₁-C₈ alkyl, C₁-C₈ alkoxy, ═O, C₁-C₈ alkyl —CO—, or (C₁-C₈ alkoxy)-CO— where any C₁-C₈ alkyl is optionally substituted by one or more hydroxy, cyano, halogen or amino groups.

3. A compound according to claim 1, wherein:
Ar is phenyl substituted by at least one substituent selected from azido substituted C₁-C₈ alkyl, C₁-C₈ alkyl-NR¹¹R^{12a}, C₁-C₈ alkyl-OR^{12a}, C₁-C₈ alkyl-SR^{12a}, wherein R^{12a} is selected from —(CR¹³₂)ₙR¹⁴, —CO—(CR¹³₂)ₙR¹⁴, —SO₂—(CR¹³₂)ₙR¹⁴;

4. A compound according to claim 1 wherein X is C═O.

5. A compound according to claim 1 wherein R^{x} is a group selected from C₁-C₈ alkyl, C₃-C₈ cycloalkyl or a saturated monocyclic 4- to 7-membered ring comprising one or more heteroatoms selected from nitrogen, oxygen and sulphur, wherein any C₃-C₈ cycloalkyl group or saturated monocyclic 4- to 7-membered ring is optionally substituted by one or more groups selected from hydroxy, azido, cyano, amino, halogen, —CONH₂—, C₁-C₈ alkyl, (C₁-C₈ alkyl)CO—, C₁-C₈alkoxy, or (C₁-C₈ alkoxy)-CO—, and any C₁-C₈ alkyl, (C₁-C₈ alkyl)CO—, C₁-C₈ alkoxy, or (C₁-C₈ alkoxy)-CO— group is itself optionally substituted by one or more substituents selected from hydroxy, azido, cyano, amino, halogen or phenyl.

6. A compound according to claim 1 wherein R^{x} is a saturated monocyclic 4- to 7-membered ring comprising one or more heteroatoms selected from nitrogen, oxygen and sulphur, which ring is substituted by one or more C₁-C₈ alkyl, (C₁-C₈ alkyl)CO—, C₁-C₈ alkoxy, or (C₁-C₈ alkoxy)-CO— groups, each of which groups is optionally substituted with one or more substituents selected from hydroxy, azido, cyano, amino, halogen, —CONH₂, C₁-C₈ alkoxy, (C₁-C₈ alkoxy)-CO— or phenyl.

7. A compound according to claim 1 wherein R^{x} is a group Ar.

8. A compound according to claim 1 wherein Ar is phenyl optionally substituted by one or more groups, which may be the same or different, selected from halogen, hydroxy, cyano, C₁-C₈ alkoxy, CO₂R⁸, CONR⁹R¹⁰, C₁-C₈ alkyl-NR⁸—C₁-C₈ alkyl, C₁-C₈ alkyl-CONR⁸—C₁-C₈ alkyl, C₁-C₈ alkyl-CONR⁹R¹⁰, NR⁸COC₁-C₈ alkyl, C₁-C₈ thioalkyl, C₁-C₈ alkyl (itself optionally substituted by one or more hydroxy, azido or cyano groups or fluorine atoms), C₁-C₈ alkyl-NR¹¹R¹², C₁-C₈ alkyl-OR¹², C₁-C₈ alkyl-SR¹².

9. A compound according to claim 1 wherein Ar is phenyl substituted by at least one substituent selected from azido substituted C₁-C₈ alkyl, C₁-C₈ alkyl-NR¹¹R^{12a}, C₁-C₈alkyl-OR^{12a}, C₁-C₈alkyl-SR^{12a}, wherein R^{12a} is selected from —(CR¹³₂)ₙR¹⁴, —CO—(CR¹³₂)ₙR¹⁴, —SO₂—(CR¹³₂)ₙR¹⁴ provided that Ar is not phenyl substituted by one or more groups selected from C₁-C₈ alkyl-NR¹¹—C₁-C₈ alkyl, C₁-C₈ alkyl-O—C₁-C₈ alkyl or C₁-C₆ alkanoyloxy C₁-C₆ alkyl.

10. A compound according to claim 9 wherein Ar is phenyl substituted by one or more —CH₂NR¹¹R¹² groups.

11. A compound according to claim 10 wherein R¹¹ and R¹² together with the nitrogen atom to which they are attached form a 4- to 10-membered saturated or unsaturated heterocyclic ring system optionally containing one or more additional heteroatoms selected from oxygen, sulphur or nitrogen, the ring itself being optionally substituted by one or more hydroxy, hydroxy(C₁-C₈)alkyl, C₁-C₈ alkyl(which may itself optionally be substituted by a 4- to 7-membered saturated or unsaturated heterocyclic ring system optionally containing a further oxygen, sulphur or nitrogen atom, the ring being optionally substituted by one or more hydroxy, hydroxy(C₁-C₈)alkyl, C₁-C₈ alkyl, nitro, —CONH₂ groups), nitro, cyano, —CONH₂, amino, ═O or —COOH groups or by a saturated monocyclic 4- to 7-membered ring, which ring may optionally comprise one or more heteroatoms selected from nitrogen, oxygen and sulphur and which may be optionally substituted by one or more substituents selected from C₁-C₈ alkyl, C₁-C₈ alkoxy or (C₁-C₈ alkoxy)-CO—.

12. A compound according to claim 1 wherein R¹ and R² independently represent C₁-C₈ alkoxy, Y(CR³₂)ₚNR⁴R⁵, Y(CR³₂)ₚCONR⁴R⁵, Y(CR³₂)ₚCO₂R⁶, Y(CR³₂)ₚOR⁶, Y(CR³₂)pOCOR⁶, Y(CR³₂)pR⁶.

13. A compound according to claim 12 wherein R¹ and R² are both C₁-C₈ alkoxy, or one of R¹ and R² is C₁-C₈ alkoxy and the other is Y(CR³₂)ₚNR⁴R⁵, Y(CR³₂)ₚOR⁶ or Y(CR³₂)ₚR⁶.

14. A compound according to claim 1 which is
6,7-diethoxy-4-{[2-ethyl-3-(1H-imidazol-1-ylmethyl)phenyl]amino}quinoline-3-carboxamide
6,7-diethoxy-4-{[2-methyl-3-(1H-1,2,4-triazol-1-ylmethyl)phenyl]amino}quinoline-3-carboxamide
6,7-diethoxy-4-{[2-ethyl-3-(morpholin-4-ylmethyl)phenyl]amino}quinoline-3-carboxamide
6,7-diethoxy-4-{[3-(1H-imidazol-1-ylmethyl)-2-methylphenyl]amino}quinoline-3-carboxamide
4-{[3-(azidomethyl)-2-methylphenyl]amino}-6,7-diethoxyquinoline-3-carboxamide
6,7-diethoxy-4-{[2-methyl-3-(4H-1,2,4-triazol-4-ylmethyl)phenyl]amino}quinoline-3-carboxamide
4-{[3-({[4-(aminosulfonyl)benzyl]amino}methyl)-2-ethylphenyl]amino}-6,7-dimethoxyquinoline-3-carboxamide
4-({2-ethyl-3-[(1H-1,2,4-triazol-5-ylamino)methyl]phenyl}amino)-6,7-dimethoxyquinoline-3-carboxamide
4-{[2-ethyl-3-(1H-imidazol-1-ylmethyl)phenyl]amino}-6,7-dimethoxyquinoline-3-carboxamide
6,7-diethoxy-4-(2-ethyl-3-[(pyrimidin-2-ylamino)methyl]phenylamino)quinoline-3-carboxamide
6,7-diethoxy-4-[(2-ethyl-3-{[(2-hydroxycyclohexyl)amino]methyl}phenyl)amino]quinoline-3-carboxamide
6,7-diethoxy-4-[(2-ethyl-3-{[(3-thienylmethyl)amino]methyl}phenyl)amino]quinoline-3-carboxamide
6,7-diethoxy-4-({2-ethyl-3-[(1H-imidazol-2-ylthio)methyl]phenyl}amino)quinoline-3-carboxamide
6,7-diethoxy-4-{[2-ethyl-3-(thiomorpholin-4-ylmethyl)phenyl]amino}quinoline-3-carboxamide
6,7-diethoxy-4-[(2-ethyl-3-{[(3-thienylmethyl)amino]methyl}phenyl)amino]quinoline-3-carboxamide
4-({2-ethyl-3-[(4-nitro-1H-imidazol-1-yl)methyl]phenyl}amino)-6,7-dimethoxyquinoline-3-carboxamide
4-[(2-ethyl-3-{[4-(hydroxymethyl)-1H-imidazol-1-yl]methyl}phenyl)amino]-6,7-dimethoxyquinoline-3-carboxamide
4-({2-ethyl-3-[(2-methyl-1H-imidazol-1-yl)methyl]phenyl}amino)-6,7-dimethoxyquinoline-3-carboxamide
1-(3-{[3-(aminocarbonyl)-6,7-dimethoxyquinolin-4-yl]amino}-2-ethylbenzyl)-1H-imidazole-4-carboxylic acid
4-({3-[(cyclopentylamino)methyl]-2-ethylphenyl}amino)-6,7-dimethoxyquinoline-3-carboxamide
4-{[2-ethyl-3-({[2-(1H-imidazol-4-yl)ethyl]amino}methyl)phenyl]amino}-6,7-dimethoxyquinoline-3-carboxamide
4-[(2-ethyl-3-{[(2-hydroxy-1,1-dimethylethyl)amino]methyl}phenyl)amino]-6,7-dimethoxyquinoline-3-carboxamide
4-({2-ethyl-3-[(1,3-thiazol-2-ylamino)methyl]phenyl}amino)-6,7-dimethoxyquinoline-3-carboxamide
4-[(2-ethyl-3-{[(2-hydroxypropyl)amino]methyl}phenyl)amino]-6,7-dimethoxyquinoline-3-carboxamide
4-[(2-ethyl-3-{[(2-hydroxy-2-phenylethyl)amino]methyl}phenyl)amino]-6,7-dimethoxyquinoline-3-carboxamide bis(trifluoroacetate)
4-{[2-ethyl-3-({[4-(methylsulfonyl)benzyl]amino}methyl)phenyl]amino}-6,7-dimethoxyquinoline-3-carboxamide
4-({3-[(benzylamino)methyl]-2-ethylphenyl}amino)-6,7-dimethoxyquinoline-3-carboxamide
4-({2-ethyl-3-[(3-methyl-2,5-dioxoimidazolidin-1-yl)methyl]phenyl}amino)-6,7-dimethoxyquinoline-3-carboxamide
4-({2-ethyl-3-[(1H-tetrazol-5-ylamino)methyl]phenyl}amino)-6,7-dimethoxyquinoline-3-carboxamide
4-({3-[(5-amino-1H-tetrazol-1-yl)methyl]-2-ethylphenyl}amino)-6,7-dimethoxyquinoline-3-carboxamide
4-{[2-ethyl-3-({[2-(2-oxoimidazolidin-1-yl)ethyl]amino}methyl)phenyl]amino}-6,7-dimethoxyquinoline-3-carboxamide
4-{[2-ethyl-3-({[(2S)-2-hydroxycyclohexyl]amino}methyl)phenyl]amino}-6,7-dimethoxyquinoline-3-carboxamide
4-({2-ethyl-3-[(piperidin-4-ylamino)methyl]phenyl}amino)-6,7-dimethoxyquinoline-3-carboxamide
4-{[2-ethyl-3-({[(1R)-1-(hydroxymethyl)-3-methyl butyl]amino}methyl)phenyl]amino}-6,7-dimethoxyquinoline-3-carboxamide
6,7-diethoxy-4-[(2-ethyl-3-{[4-(3-methoxyphenyl)piperazin-1-yl]methyl}phenyl)amino]quinoline-3-carboxamide
6,7-diethoxy-4-[(2-ethyl-3-{[4-(hydroxymethyl)piperidin-1-yl]methyl}phenyl)amino]quinoline-3-carboxamide
6,7-diethoxy-4-[(2-ethyl-3-{[2-(hydroxymethyl)piperidin-1-yl]methyl}phenyl)amino]quinoline-3-carboxamide
4-{[3-(1,4′-bipiperidin-1′-ylmethyl)-2-ethylphenyl]amino}-6,7-diethoxyquinoline-3-carboxamide
4-[(3-{[4-(aminocarbonyl)piperidin-1-yl]methyl}-2-ethyl phenyl)amino]-6,7-diethoxyquinoline-3-carboxamide
4-[(3-{[4-(2-cyanophenyl)piperazin-1-yl]methyl}-2-ethylphenyl)amino]-6,7-diethoxyquinoline-3-carboxamide
4-[(3-{[4-(5-cyanopyridin-2-yl)piperazin-1-yl]methyl}-2-ethyl phenyl)amino]-6,7-diethoxyquinoline-3-carboxamide
6,7-diethoxy-4-[(2-ethyl-3-{[(3-furylmethyl)amino]methyl}phenyl)amino]quinoline-3-carboxamide
6,7-diethoxy-4-[(2-ethyl-3-{[4-(2-hydroxyethyl)piperazin-1-yl]methyl}phenyl)amino]quinoline-3-carboxamide
6,7-diethoxy-4-({2-ethyl-3-[(4-hydroxypiperidin-1-yl)methyl]phenyl}amino)quinoline-3-carboxamide
4-{[3-({[2-(1,3-benzodioxol-5-yl)ethyl]amino}methyl)-2-ethylphenyl]amino}-6,7-diethoxyquinoline-3-carboxamide
6,7-diethoxy-4-{[2-ethyl-3-({[2-(2-thienyl)ethyl]amino}methyl)phenyl]amino}quinoline-3-carboxamide
4-{[3-({[(2,5-dimethyl-3-furyl)methyl]amino}methyl)-2-ethylphenyl]amino}-6,7-diethoxyquinoline-3-carboxamide
6,7-diethoxy-4-{[2-ethyl-3-({[3-(2-oxopyrrolidin-1-yl)propyl]amino}methyl)phenyl]amino}quinoline-3-carboxamide
4-{[3-({[2-(3-chlorophenyl)ethyl]amino}methyl)-2-ethylphenyl]amino}-6,7-diethoxyquinoline-3-carboxamide
4-{[3-({[2-(4-chlorophenyl)ethyl]amino}methyl)-2-ethylphenyl]amino}-6,7-diethoxyquinoline-3-carboxamide
4-{[3-({[2-(2-chlorophenyl)ethyl]amino}methyl)-2-ethylphenyl]amino}-6,7-diethoxyquinoline-3-carboxamide
6,7-diethoxy-4-[(2-ethyl-3-{[(2-hydroxy-2-phenylethyl)amino]methyl}phenyl)amino]quinoline-3-carboxamide
4-({3-[(cyclopentylamino)methyl]-2-ethylphenyl}amino)-6,7-diethoxyquinoline-3-carboxamide
6,7-diethoxy-4-{[2-ethyl-3-({[2-(1H-imidazol-4-yl)ethyl]amino}methyl)phenyl]amino}quinoline-3-carboxamide
6,7-diethoxy-4-[(2-ethyl-3-{[4-(2-morpholin-4-ylethyl)piperazin-1-yl]methyl}phenyl)amino]quinoline-3-carboxamide
4-{[3-({[(2,2-dimethyl-1,3-dioxolan-4-yl)methyl]amino}methyl)-2-ethylphenyl]amino}-6,7-diethoxyquinoline-3-carboxamide
6,7-diethoxy-4-({2-ethyl-3-[(1,3-thiazol-2-ylamino)methyl]phenyl}amino)quinoline-3-carboxamide
6,7-diethoxy-4-{[2-ethyl-3-(1,3-thiazolidin-3-ylmethyl)phenyl]amino}quinoline-3-carboxamide
6,7-diethoxy-4-[(2-ethyl-3-{[(2-pyridin-2-ylethyl)amino]methyl}phenyl)amino]quinoline-3-carboxamide
6,7-diethoxy-4-({2-ethyl-3-[(1H-1,2,4-triazol-3-ylamino)methyl]phenyl}amino)quinoline-3-carboxamide
6,7-diethoxy-4-{[2-ethyl-3-({[4-(2-thienyl)benzyl]amino}methyl)phenyl]amino}quinoline-3-carboxamide
4-{[3-({[4-(aminosulfonyl)benzyl]amino}methyl)-2-ethylphenyl]amino}-6,7-diethoxyquinoline-3-carboxamide
6,7-diethoxy-4-{[2-ethyl-3-({[2-(1H-indol-3-yl)ethyl]amino}methyl)phenyl]amino}quinoline-3-carboxamide
6,7-diethoxy-4-{[2-ethyl-3-({[3-(4-methylpiperazin-1-yl)propyl]amino}methyl)phenyl]amino}quinoline-3-carboxamide
6,7-diethoxy-4-[(2-ethyl-3-{[(1-ethylpiperidin-3-yl)amino]methyl}phenyl)amino]quinoline-3-carboxamide
6,7-diethoxy-4-[(2-ethyl-3-{[4-(pyridin-4-ylmethyl)piperazin-1-yl]methyl}phenyl)amino]quinoline-3-carboxamide
6,7-diethoxy-4-[(2-ethyl-3-{[(pyridin-4-ylmethyl)amino]methyl}phenyl)amino]quinoline-3-carboxamide
6,7-diethoxy-4-[(2-ethyl-3-{[(pyridin-3-ylmethyl)amino]methyl}phenyl)amino]quinoline-3-carboxamide
4-({3-[(benzylamino)methyl]-2-ethylphenyl}amino)-6,7-diethoxyquinoline-3-carboxamide
6,7-diethoxy-4-[(2-ethyl-3-{[(2-furylmethyl)amino]methyl}phenyl)amino]quinoline-3-carboxamide
6,7-diethoxy-4-[(2-ethyl-3-{[(2-methoxyethyl)amino]methyl}phenyl)amino]quinoline-3-carboxamide
6,7-diethoxy-4-[(2-ethyl-3-{[(2-hydroxypropyl)amino]methyl}phenyl)amino]quinoline-3-carboxamide
6,7-diethoxy-4-{[2-ethyl-3-({[4-(1H-pyrazol-1-yl)benzyl]amino}methyl)phenyl]amino}quinoline-3-carboxamide
4-({3-[({2-[4-(aminosulfonyl)phenyl]ethyl}amino)methyl]-2-ethyl phenyl}amino)-6,7-diethoxyquinoline-3-carboxamide
6,7-diethoxy-4-{[2-ethyl-3-({[2-(1-methylpyrrolidin-2-yl)ethyl]amino}methyl)phenyl]amino}quinoline-3-carboxamide
4-[(3-{[(4-chlorobenzyl)amino]methyl}-2-ethyl phenyl)amino]-6,7-diethoxyquinoline-3-carboxamide
4-[(3-{[(1-benzyl piperidin-4-yl)amino]methyl}-2-ethylphenyl)amino]-6,7-diethoxyquinoline-3-carboxamide
6,7-diethoxy-4-[(2-ethyl-3-{[(3-methoxybenzyl)amino]methyl}phenyl)amino]quinoline-3-carboxamide
6,7-diethoxy-4-[(2-ethyl-3-{[(4-methoxybenzyl)amino]methyl}phenyl)amino]quinoline-3-carboxamide
6,7-diethoxy-4-{[2-ethyl-3-({[3-(1H-imidazol-1-yl)propyl]amino}methyl)phenyl]amino}quinoline-3-carboxamide
6,7-diethoxy-4-{[2-ethyl-3-({[(1R,2S)-2-hydroxy-2,3-dihydro-1H-inden-1-yl]amino}methyl)phenyl]amino}quinoline-3-carboxamide bis(trifluoroacetate) (salt)
6,7-diethoxy-4-{[2-ethyl-3-({[2-hydroxy-1-(1H-indol-2-ylmethyl)ethyl]amino}methyl)phenyl]amino}quinoline-3-carboxamide bis(trifluoroacetate) (salt)
6,7-diethoxy-4-{[2-ethyl-3-({[(1R)-2-hydroxy-1-phenylethyl]amino}methyl)phenyl]amino}quinoline-3-carboxamide bis(trifluoroacetate) (salt)
6,7-Diethoxy-4-{2-ethyl-3-[(2-hydroxy-1-methylcarbamoyl-propylamino)-methyl]-phenylamino}-quinoline-3-carboxylic acid amide
6,7-diethoxy-4-{[2-ethyl-3-({[(1R,2S)-2-hydroxy-1-(hydroxymethyl)propyl]amino}methyl)phenyl]amino}quinoline-3-carboxamide
6,7-diethoxy-4-{[2-ethyl-3-({[(1R,2R)-2-hydroxy-1-(hydroxymethyl)propyl]amino}methyl)phenyl]amino}quinoline-3-carboxamide
methyl N-(3-{[3-(aminocarbonyl)-6,7-diethoxyquinolin-4-yl]amino}-2-ethyl benzyl)serinate bis(trifluoroacetate)
6,7-diethoxy-4-{[2-ethyl-3-({[2-hydroxy-1-(hydroxymethyl)ethyl]amino}methyl)phenyl]amino}quinoline-3-carboxamide
6,7-diethoxy-4-{[2-ethyl-3-({[1-(hydroxymethyl)-3-methyl butyl]amino}methyl)phenyl]amino}quinoline-3-carboxamide
6,7-diethoxy-4-[(2-ethyl-3-{[(2-pyrrolidin-1-ylethyl)amino]methyl}phenyl)amino]quinoline-3-carboxamide
6,7-diethoxy-4-{[2-ethyl-3-({[(1S,2R)-2-hydroxy-1-(hydroxymethyl)propyl]amino}methyl)phenyl]amino}quinoline-3-carboxamide
6,7-diethoxy-4-{[2-ethyl-3-({[(1S)-1-(hydroxymethyl)-3-methyl butyl]amino}methyl)phenyl]amino}quinoline-3-carboxamide
6,7-diethoxy-4-{[2-ethyl-3-({[1-(hydroxymethyl)butyl]amino}methyl)phenyl]amino}quinoline-3-carboxamide
4-{3-[(1-Carbamoyl-2-hydroxy-propylamino)-methyl]-2-ethyl-phenylamino}-6,7-diethoxy-quinoline-3-carboxylic acid amide
6,7-diethoxy-4-[(2-ethyl-3-{[[(1R,2R)-2-hydroxy-1-methyl-2-phenylethyl](methyl)amino]methyl}phenyl)amino]quinoline-3-carboxamide
6,7-diethoxy-4-[(2-ethyl-3-{[(2-hydroxy-1-methyl-2-phenylethyl)amino]methyl}phenyl)amino]quinoline-3-carboxamide
4-{[3-({[2-(3,4-dihydroxyphenyl)-2-hydroxyethyl]amino}methyl)-2-ethylphenyl]amino}-6,7-diethoxyquinoline-3-carboxamide
6,7-diethoxy-4-[(2-ethyl-3-{[(2-hydroxypropyl)amino]methyl}phenyl)amino]quinoline-3-carboxamide
6,7-diethoxy-4-[(2-ethyl-3-{[(2-hydroxy-1-methylethyl)amino]methyl}phenyl)amino]quinoline-3-carboxamide
6,7-diethoxy-4-[(2-ethyl-3-{[(2-hydroxyethyl)amino]methyl}phenyl)amino]quinoline-3-carboxamide
4-[(3-{[(2,3-dihydroxypropyl)amino]methyl}-2-ethylphenyl)amino]-6,7-diethoxyquinoline-3-carboxamide
6,7-diethoxy-4-{[2-ethyl-3-({[2-(hydroxymethyl)phenyl]amino}methyl)phenyl]amino}quinoline-3-carboxamide
4-{[3-({[(1S)-1-benzyl-2-hydroxyethyl]amino}methyl)-2-ethylphenyl]amino}-6,7-diethoxyquinoline-3-carboxamide bis(trifluoroacetate)
4-{[3-({[2-(dimethylamino)ethyl]amino}methyl)-2-ethylphenyl]amino}-6,7-diethoxyquinoline-3-carboxamide
6,7-diethoxy-4-{[2-ethyl-3-({[4-(methylsulfonyl)phenyl]amino}methyl)phenyl]amino}quinoline-3-carboxamide
6,7-diethoxy-4-{[2-ethyl-3-({[(1S)-2-hydroxy-1-phenylethyl]amino}methyl)phenyl]amino}quinoline-3-carboxamide
6,7-diethoxy-4-[(2-ethyl-3-{[(2R)-2-(hydroxymethyl)pyrrolidin-1-yl]methyl}phenyl)amino]quinoline-3-carboxamide
6,7-diethoxy-4-{[2-ethyl-3-({[(1S,2S)-2-hydroxy-1-(hydroxymethyl)-2-phenylethyl]amino}methyl)phenyl]amino}quinoline-3-carboxamide
6,7-diethoxy-4-[(2-ethyl-3-{[(2-morpholin-4-ylethyl)amino]methyl}phenyl)amino]quinoline-3-carboxamide
6,7-diethoxy-4-{[2-ethyl-3-({[(1R,2S)-2-hydroxy-2-(4-hydroxyphenyl)-1-methylethyl]amino}methyl)phenyl]amino}quinoline-3-carboxamide
6,7-diethoxy-4-{[2-ethyl-3-({[(1R,2R)-2-hydroxy-1-(hydroxymethyl)-2-phenylethyl]amino}methyl)phenyl]amino}quinoline-3-carboxamide
6,7-Diethoxy-4-{2-ethyl-3-[(2-hydroxy-1-hydroxymethyl-2-phenyl-ethylamino)-methyl]-phenylamino}-quinoline-3-carboxylic acid amide
4-[(3-{[(2-cyanoethyl)amino]methyl}-2-ethylphenyl)amino]-6,7-diethoxyquinoline-3-carboxamide
6,7-diethoxy-4-{[2-ethyl-3-({[1-(hydroxymethyl)-2-methylpropyl]amino}methyl)phenyl]amino}quinoline-3-carboxamide
6,7-diethoxy-4-{[2-ethyl-3-({[4-(methylsulfonyl)benzyl]amino}methyl)phenyl]amino}quinoline-3-carboxamide
tert-butyl (3-{[3-(aminocarbonyl)-6,7-diethoxyquinolin-4-yl]amino}-2-ethylbenzyl)carbamate
4-{[3-(aminomethyl)-2-ethylphenyl]amino}-6,7-diethoxyquinoline-3-carboxamide
4-{[3-(aminomethyl)-2-methylphenyl]amino}-6,7-diethoxyquinoline-3-carboxamide
6,7-diethoxy-4-({2-ethyl-3-[(L-tyrosylamino)methyl]phenyl}amino)quinoline-3-carboxamide
6,7-diethoxy-4-{[3-({[(ethylamino)carbonyl]amino}methyl)-2-methylphenyl]amino}quinoline-3-carboxamide
4-({3-[(acetylamino)methyl]-2-methylphenyl}amino)-6,7-diethoxyquinoline-3-carboxamide
6,7-diethoxy-4-({2-methyl-3-[({[(4-methyl-2,5-dioxoimidazolidin-4-yl)methyl]sulfonyl}amino)methyl]phenyl}amino)quinoline-3-carboxamide
4-({3-[(acetylamino)methyl]-2-ethylphenyl}amino)-6,7-dimethoxyquinoline-3-carboxamide
4-{[2-ethyl-3-({[(ethylamino)carbonyl]amino}methyl)phenyl]amino}-6,7-dimethoxyquinoline-3-carboxamide
4-[(2-ethyl-3-{[(methylsulfonyl)amino]methyl}phenyl)amino]-6,7-dimethoxyquinoline-3-carboxamide
4-({2-ethyl-3-[(L-valylamino)methyl]phenyl}amino)-6,7-dimethoxyquinoline-3-carboxamide
4-[(3-{[(3-cyclohexyl-L-alanyl)amino]methyl}-2-ethylphenyl)amino]-6,7-dimethoxyquinoline-3-carboxamide
6,7-diethoxy-4-({2-ethyl-3-[(L-methionylamino)methyl]phenyl}amino)quinoline-3-carboxamide
6,7-diethoxy-4-({2-ethyl-3-[(L-prolylamino)methyl]phenyl}amino)quinoline-3-carboxamide
6,7-diethoxy-4-({2-ethyl-3-[(L-threonylamino)methyl]phenyl}amino)quinoline-3-carboxamide
N˜1˜-(3-{[3-(aminocarbonyl)-6,7-diethoxyquinolin-4-yl]amino}-2-ethylbenzyl)-L-alpha-glutamine
6,7-diethoxy-4-({2-ethyl-3-[(L-valylamino)methyl]phenyl}amino)quinoline-3-carboxamide
4-({3-[(L-arginylamino)methyl]-2-ethylphenyl}amino)-6,7-diethoxyquinoline-3-carboxamide
4-({3-[(L-alanylamino)methyl]-2-ethylphenyl}amino)-6,7-diethoxyquinoline-3-carboxamide
6,7-diethoxy-4-({2-ethyl-3-[(D-serylamino)methyl]phenyl}amino)quinoline-3-carboxamide
4-[(3-{[(3-cyclohexyl-L-alanyl)amino]methyl}-2-ethylphenyl)amino]-6,7-diethoxyquinoline-3-carboxamide
6,7-diethoxy-4-{[2-ethyl-3-({[(4S)-1,3-thiazolidin-4-ylcarbonyl]amino}methyl)phenyl]amino}quinoline-3-carboxamide
6,7-diethoxy-4-{[2-ethyl-3-({[(4R)-4-hydroxy-L-prolyl]amino}methyl)phenyl]amino}quinoline-3-carboxamide
6,7-diethoxy-4-({2-ethyl-3-[(D-leucylamino)methyl]phenyl}amino)quinoline-3-carboxamide
N˜1˜-(3-{[3-(aminocarbonyl)-6,7-diethoxyquinolin-4-yl]amino}-2-ethylbenzyl)-L-aspartamide
6,7-diethoxy-4-{[2-ethyl-3-({[(2S)-piperidin-2-ylcarbonyl]amino}methyl)phenyl]amino}quinoline-3-carboxamide
4-[(3-{[(3-cyclohexyl-D-alanyl)amino]methyl}-2-ethylphenyl)amino]-6,7-diethoxyquinoline-3-carboxamide
6,7-diethoxy-4-{[2-ethyl-3-({[(2R)-piperidin-2-ylcarbonyl]amino}methyl)phenyl]amino}quinoline-3-carboxamide
4-{[3-({[(2S)-2-aminopent-4-enoyl]amino}methyl)-2-ethyl phenyl]amino}-6,7-diethoxyquinoline-3-carboxamide
4-{[3-({[(2S)-azetidin-2-ylcarbonyl]amino}methyl)-2-ethyl phenyl]amino}-6,7-diethoxyquinoline-3-carboxamide
6,7-diethoxy-4-[(2-ethyl-3-{[(5-methyl-L-norleucyl)amino]methyl}phenyl)amino]quinoline-3-carboxamide
6,7-diethoxy-4-{[2-ethyl-3-({[(4R)-1,3-thiazolidin-4-ylcarbonyl]amino}methyl)phenyl]amino}quinoline-3-carboxamide
6,7-diethoxy-4-[(2-ethyl-3-{[(4-nitro-D-phenylalanyl)amino]methyl}phenyl)amino]quinoline-3-carboxamide
4-{[3-({[(1-amino-2,3-dihydro-1H-inden-1-yl)carbonyl]amino}methyl)-2-ethylphenyl]amino}-6,7-diethoxyquinoline-3-carboxamide
4-{[3-({[(1-aminocyclohexyl)carbonyl]amino}methyl)-2-ethylphenyl]amino}-6,7-diethoxyquinoline-3-carboxamide
6,7-diethoxy-4-{[2-ethyl-3-({[(3R)-1,2,3,4-tetrahydroisoquinolin-3-ylcarbonyl]amino}methyl)phenyl]amino}quinoline-3-carboxamide
4-{[3-({[(2R)-2-amino-4-phenylbutanoyl]amino}methyl)-2-ethyl phenyl]amino}-6,7-diethoxyquinoline-3-carboxamide
6,7-diethoxy-4-{[2-ethyl-3-({[(3S)-1,2,3,4-tetrahydroisoquinolin-3-ylcarbonyl]amino}methyl)phenyl]amino}quinoline-3-carboxamide
6,7-diethoxy-4-[(2-ethyl-3-{[(4-piperidin-4-yl-L-prolyl)amino]methyl}phenyl)amino]quinoline-3-carboxamide
4-[(3-{[(3-amino-L-alanyl)amino]methyl}-2-ethyl phenyl)amino]-6,7-diethoxyquinoline-3-carboxamide
6,7-diethoxy-4-({2-ethyl-3-[(D-phenylalanylamino)methyl]phenyl}amino)quinoline-3-carboxamide
4-{[3-({[(2S)-2-amino-4-phenylbutanoyl]amino}methyl)-2-ethylphenyl]amino}-6,7-diethoxyquinoline-3-carboxamide
6,7-diethoxy-4-{[2-ethyl-3-({[(3S)-piperidin-3-ylcarbonyl]amino}methyl)phenyl]amino}quinoline-3-carboxamide
6,7-diethoxy-4-{[2-ethyl-3-({[(3R)-piperidin-3-ylcarbonyl]amino}methyl)phenyl]amino}quinoline-3-carboxamide
4-{[3-({[(2S)-2-amino-2-phenylacetyl]amino}methyl)-2-ethyl phenyl]amino}-6,7-diethoxyquinoline-3-carboxamide
6,7-diethoxy-4-({2-ethyl-3-[(L-leucylamino)methyl]phenyl}amino)quinoline-3-carboxamide
6,7-diethoxy-4-({2-ethyl-3-[(D-prolylamino)methyl]phenyl}amino)quinoline-3-carboxamide
4-{[3-({[(2S)-2,5-dihydro-1H-pyrrol-2-ylcarbonyl]amino}methyl)-2-ethylphenyl]amino}-6,7-diethoxyquinoline-3-carboxamide
6,7-diethoxy-4-({2-ethyl-3-[(glycylamino)methyl]phenyl}amino)quinoline-3-carboxamide
4-{[3-({[2-amino-4-(methylsulfinyl)butanoyl]amino}methyl)-2-ethylphenyl]amino}-6,7-diethoxyquinoline-3-carboxamide
6,7-diethoxy-4-{[2-ethyl-3-({[3-(2-furyl)-L-alanyl]amino}methyl)phenyl]amino}quinoline-3-carboxamide
6,7-diethoxy-4-[(2-ethyl-3-{[(3-pyridin-2-yl-L-alanyl)amino]methyl}phenyl)amino]quinoline-3-carboxamide
6,7-diethoxy-4-{[2-ethyl-3-({[3-(2-thienyl)-L-alanyl]amino}methyl)phenyl]amino}quinoline-3-carboxamide
6,7-diethoxy-4-{[2-ethyl-3-({[3-(1,3-thiazol-4-yl)-L-alanyl]amino}methyl)phenyl]amino}quinoline-3-carboxamide
4-{[3-({[(2S)-2-amino-2-cyclopentylacetyl]amino}methyl)-2-ethyl phenyl]amino}-6,7-diethoxyquinoline-3-carboxamide
4-{[3-({[(2S)-2-aminopent-4-ynoyl]amino}methyl)-2-ethylphenyl]amino}-6,7-diethoxyquinoline-3-carboxamide
6,7-diethoxy-4-({2-ethyl-3-[(L-norvalylamino)methyl]phenyl}amino)quinoline-3-carboxamide
4-{[3-({[(2R)-2-amino-2-phenylacetyl]amino}methyl)-2-ethylphenyl]amino}-6,7-diethoxyquinoline-3-carboxamide
6,7-diethoxy-4-{[2-ethyl-3-({[(4R)-4-hydroxy-D-prolyl]amino}methyl)phenyl]amino}quinoline-3-carboxamide
4-({3-[(beta-alanylamino)methyl]-2-ethylphenyl}amino)-6,7-diethoxyquinoline-3-carboxamide
6,7-diethoxy-4-[(2-ethyl-3-{[(3-pyridin-3-yl-L-alanyl)amino]methyl}phenyl)amino]quinoline-3-carboxamide
6,7-diethoxy-4-[(2-ethyl-3-{[(3-pyridin-3-yl-D-alanyl)amino]methyl}phenyl)amino]quinoline-3-carboxamide
4-{[3-({[N˜5˜-(aminocarbonyl)-L-ornithyl]amino}methyl)-2-ethylphenyl]amino}-6,7-diethoxyquinoline-3-carboxamide
6,7-diethoxy-4-[(2-ethyl-3-{[(5-methyl-D-norleucyl)amino]methyl}phenyl)amino]quinoline-3-carboxamide
4-[(3-{[(2,3-dihydro-1H-isoindol-1-ylcarbonyl)amino]methyl}-2-ethylphenyl)amino]-6,7-diethoxyquinoline-3-carboxamide
6,7-diethoxy-4-({2-ethyl-3-[(L-isoleucylamino)methyl]phenyl}amino)quinoline-3-carboxamide
6,7-diethoxy-4-({2-ethyl-3-[(D-valylamino)methyl]phenyl}amino)quinoline-3-carboxamide 4-{[3-({[(1-aminocyclopentyl)carbonyl]amino}methyl)-2-ethyl phenyl]amino}-6,7-diethoxyquinoline-3-carboxamide
4-{[2-ethyl-3-(hydroxymethyl)phenyl]amino}-7-{3-[isobutyryl(isopropyl)amino]propoxy}-6-methoxyquinoline-3-carboxamide
7-{3-[acetyl(isopropyl)amino]propoxy}-4-{[2-ethyl-3-(hydroxymethyl)phenyl]amino}-6-methoxyquinoline-3-carboxamide
6-[2-(acetylamino)ethoxy]-4-[(2-ethylphenyl)amino]-7-methoxyquinoline-3-carboxamide
6-{2-[acetyl(methyl)amino]ethoxy}-4-[(2-ethylphenyl)amino]-7-methoxyquinoline-3-carboxamide
6-{2-[acetyl(isopropyl)amino]ethoxy}-4-[(2-ethylphenyl)amino]-7-methoxyquinoline-3-carboxamide
4-[(2-ethylphenyl)amino]-6-{2-[isobutyryl(methyl)amino]ethoxy}-7-methoxyquinoline-3-carboxamide
4-[(2-ethylphenyl)amino]-6-{2-[isobutyryl(isopropyl)amino]ethoxy}-7-methoxyquinoline-3-carboxamide
7-{3-[acetyl(methyl)amino]propoxy}-4-{[2-ethyl-3-(hydroxymethyl)phenyl]amino}-6-methoxyquinoline-3-carboxamide
4-{[2-ethyl-3-(hydroxymethyl)phenyl]amino}-7-{3-[isobutyryl(methyl)amino]propoxy}-6-methoxyquinoline-3-carboxamide
7-{3-[acetyl(cyclopropyl)amino]propoxy}-4-{[2-ethyl-3-(hydroxymethyl)phenyl]amino}-6-methoxyquinoline-3-carboxamide
7-{3-[cyclopropyl(isobutyryl)amino]propoxy}-4-{[2-ethyl-3-(hydroxymethyl)phenyl]amino}-6-methoxyquinoline-3-carboxamide
7-[3-(acetylamino)propoxy]-4-{[2-ethyl-3-(hydroxymethyl)phenyl]amino}-6-methoxyquinoline-3-carboxamide
4-{[2-ethyl-3-(hydroxymethyl)phenyl]amino}-7-[3-(isobutyrylamino)propoxy]-6-methoxyquinoline-3-carboxamide
6-{2-[(cyclopropylcarbonyl)(methyl)amino]ethoxy}-4-[(2-ethylphenyl)amino]-7-methoxyquinoline-3-carboxamide
6-{2-[(cyclopropylcarbonyl)(isopropyl)amino]ethoxy}-4-[(2-ethylphenyl)amino]-7-methoxyquinoline-3-carboxamide
4-{[2-ethyl-3-(hydroxymethyl)phenyl]amino}-7-{3-[isopropyl(methylsulfonyl)amino]propoxy}-6-methoxyquinoline-3-carboxamide
4-{[2-ethyl-3-(hydroxymethyl)phenyl]amino}-6-methoxy-7-{3-[(methylsulfonyl)amino]propoxy}quinoline-3-carboxamide
tert-butyl {3-[(3-(aminocarbonyl)-4-{[2-ethyl-3-(hydroxymethyl)phenyl]amino}-6-methoxyquinolin-7-yl)oxy]propyl}isopropylcarbamate
4-{[2-ethyl-3-(hydroxymethyl)phenyl]amino}-7-(3-{isopropyl[(isopropylamino)carbonyl]amino}propoxy)-6-methoxyquinoline-3-carboxamide
7-[3-(cyclopropylamino)propoxy]-4-{[2-ethyl-3-(hydroxymethyl)phenyl]amino}-6-methoxyquinoline-3-carboxamide
6-[3-(cyclopropylamino)propoxy]-4-{[2-ethyl-3-(hydroxymethyl)phenyl]amino}-7-methoxyquinoline-3-carboxamide
7-{3-[(2-cyanoethyl)(methyl)amino]propoxy}-4-{[3-(hydroxymethyl)-2-methylphenyl]amino}-6-methoxyquinoline-3-carboxamide bis(trifluoroacetate) (salt)
4-{[3-(hydroxymethyl)-2-methylphenyl]amino}-6-methoxy-7-[3-(2-methylpiperidin-1-yl)propoxy]quinoline-3-carboxamide
7-{3-[(2-cyanoethyl)(methyl)amino]propoxy}-4-{[3-(hydroxymethyl)-2-methylphenyl]amino}-6-methoxyquinoline-3-carboxamide
4-{[3-(hydroxymethyl)-2-methylphenyl]amino}-7-[3-(3-hydroxypiperidin-1-yl)propoxy]-6-methoxyquinoline-3-carboxamide
4-{[3-(hydroxymethyl)-2-methylphenyl]amino}-7-[3-(4-hydroxypiperidin-1-yl)propoxy]-6-methoxyquinoline-3-carboxamide
6-methoxy-4-[(2-methylphenyl)amino]-7-[3-(2-methylpiperidin-1-yl)propoxy]quinoline-3-carboxamide
7-[3-(3-hydroxypiperidin-1-yl)propoxy]-6-methoxy-4-[(2-methylphenyl)amino]quinoline-3-carboxamide
7-[3-(4-hydroxypiperidin-1-yl)propoxy]-6-methoxy-4-[(2-methylphenyl)amino]quinoline-3-carboxamide
4-{[3-(hydroxymethyl)-2-methylphenyl]amino}-7-[3-(3-hydroxypyrrolidin-1-yl)propoxy]-6-methoxyquinoline-3-carboxamide
4-{[2-ethyl-3-(hydroxymethyl)phenyl]amino}-6-methoxy-7-[3-(1H-1,2,4-triazol-1-yl)propoxy]quinoline-3-carboxamide
7-[2-(cyclopropylamino)ethoxy]-4-{[3-(hydroxymethyl)-2-methylphenyl]amino}-6-methoxyquinoline-3-carboxamide
6-[2-(cyclopropylamino)ethoxy]-4-{[3-(hydroxymethyl)-2-methylphenyl]amino}-7-methoxyquinoline-3-carboxamide
6-[2-(cyclopropylamino)ethoxy]-4-[(4-ethylphenyl)amino]-7-methoxyquinoline-3-carboxamide
6-[2-(cyclopropylamino)ethoxy]-4-[(3-ethylphenyl)amino]-7-methoxyquinoline-3-carboxamide
6-[2-(cyclopropylamino)ethoxy]-7-methoxy-4-[(2-methylphenyl)amino]quinoline-3-carboxamide
6-{2-[(2-cyanoethyl)amino]ethoxy}-4-{[3-(hydroxymethyl)-2-methylphenyl]amino}-7-methoxyquinoline-3-carboxamide
6-[3-(cyclopropylamino)propoxy]-4-[(2-ethylphenyl)amino]-7-methoxyquinoline-3-carboxamide
6-{3-[(cyanomethyl)amino]propoxy}-4-[(2-ethylphenyl)amino]-7-methoxyquinoline-3-carboxamide
6-[3-(Carbamoylmethyl-amino)-propoxy]-4-(2-ethyl-phenylamino)-7-methoxy-quinoline-3-carboxylic acid amide
methyl N-[3-({3-(aminocarbonyl)-4-[(2-ethyl phenyl)amino]-7-methoxyquinolin-6-yl}oxy)propyl]glycinate
7-(3-cyanopropoxy)-4-{[2-ethyl-3-(hydroxymethyl)phenyl]amino}-6-methoxyquinoline-3-carboxamide
2-[(3-(aminocarbonyl)-4-{[2-ethyl-3-(hydroxymethyl)phenyl]amino}-6-methoxyquinolin-7-yl)oxy]ethyl acetate
6-[2-(cyclopropylamino)ethoxy]-4-[(2-ethylphenyl)amino]-7-methoxyquinoline-3-carboxamide
7-[3-(2,5-dioxopyrrolidin-1-yl)propoxy]-4-{[2-ethyl-3-(hydroxymethyl)phenyl]amino}-6-methoxyquinoline-3-carboxamide
4-{[2-ethyl-3-(hydroxymethyl)phenyl]amino}-6-methoxy-7-[3-(3-methyl-2,5-dioxoimidazolidin-1-yl)propoxy]quinoline-3-carboxamide
4-{[2-ethyl-3-(hydroxymethyl)phenyl]amino}-6-methoxy-7-[3-(3,4,4-trimethyl-2,5-dioxoimidazolidin-1-yl)propoxy]quinoline-3-carboxamide
7-(cyclopentyloxy)-4-{[2-ethyl-3-(hydroxymethyl)phenyl]amino}-6-methoxyquinoline-3-carboxamide
6-(cyclopentyloxy)-4-[(2-ethyl phenyl)amino]-7-methoxyquinoline-3-carboxamide
1-{3-[(3-(aminocarbonyl)-4-{[3-(hydroxymethyl)-2-methyl phenyl]amino}-6-methoxyquinolin-7-yl)oxy]propyl}-1-methylpyrrolidinium iodide
tert-butyl 4-[(3-(aminocarbonyl)-4-{[2-ethyl-3-(hydroxymethyl)phenyl]amino}-6-methoxyquinolin-7-yl)oxy]piperidine-1-carboxylate
tert-butyl 4-({3-(aminocarbonyl)-4-[(2-ethylphenyl)amino]-7-methoxyquinolin-6-yl}oxy)piperidine-1-carboxylate
3-(aminocarbonyl)-4-[(2-ethylphenyl)amino]-7-methoxyquinolin-6-yl propane-2-sulfonate
4-{[2-ethyl-3-(hydroxymethyl)phenyl]amino}-6-methoxy-7-(piperidin-4-yloxy)quinoline-3-carboxamide
4-[(2-ethylphenyl)amino]-7-methoxy-6-(piperidin-4-yloxy)quinoline-3-carboxamide
6-[3-(cyclopropylamino)-2-hydroxypropoxy]-4-[(2-ethylphenyl)amino]-7-methoxyquinoline-3-carboxamide
6-{3-[(2-cyanoethyl)amino]-2-hydroxypropoxy}-4-[(2-ethylphenyl)amino]-7-methoxyquinoline-3-carboxamide
4-[(2-ethylphenyl)amino]-6-[2-hydroxy-3-(2-hydroxypyrrolidin-1-yl)propoxy]-7-methoxyquinoline-3-carboxamide
4-[(2-ethylphenyl)amino]-6-(2-hydroxy-3-piperazin-1-yl propoxy)-7-methoxyquinoline-3-carboxamide
6-{[(2R)-3-(cyclopropylamino)-2-hydroxy-2-methylpropyl]oxy}-4-[(2-ethyl phenyl)amino]-7-methoxyquinoline-3-carboxamide
6-{[(2S)-3-(cyclopropylamino)-2-hydroxy-2-methylpropyl]oxy}-4-[(2-ethyl phenyl)amino]-7-methoxyquinoline-3-carboxamide
6-[3-(cyclopropylamino)-2-hydroxypropoxy]-4-{[2-ethyl-3-(hydroxymethyl)phenyl]amino}-7-methoxyquinoline-3-carboxamide
6-{[(2R)-3-(cyclopropylamino)-2-hydroxypropyl]oxy}-4-[(2-ethylphenyl)amino]-7-methoxyquinoline-3-carboxamide
6-{[(2S)-3-(cyclopropylamino)-2-hydroxypropyl]oxy}-4-[(2-ethylphenyl)amino]-7-methoxyquinoline-3-carboxamide
3-(aminocarbonyl)-4-[(2-ethylphenyl)amino]-7-methoxyquinolin-6-yl 2-methylpropanoate
6,7-diethoxy-4-[(4-methyl-1-oxo-1,2-dihydroisoquinolin-5-yl)amino]quinoline-3-carboxamide
6,7-diethoxy-4-[(4-methyl-1-oxo-1,2,3,4-tetrahydroisoquinolin-5-yl)amino]quinoline-3-carboxamide
tert-butyl 5-{[3-(aminocarbonyl)-6,7-diethoxyquinolin-4-yl]amino}-3,4-dihydroisoquinoline-2(1H)-carboxylate
6,7-diethoxy-4-(1,2,3,4-tetrahydroisoquinolin-5-ylamino)quinoline-3-carboxamide
4-{[3-(azidomethyl)-2-ethylphenyl]amino}-6-[3-(cyclopropylamino)propoxy]-7-methoxyquinoline-3-carboxamide
4-{[3-(aminomethyl)-2-ethylphenyl]amino}-6-[3-(cyclopropylamino)propoxy]-7-methoxyquinoline-3-carboxamide
4-{[3-(aminomethyl)-2-ethylphenyl]amino}-7-{3-[isobutyryl(isopropyl)amino]propoxy}-6-methoxyquinoline-3-carboxamide
4-{[3-(azidomethyl)-2-ethylphenyl]amino}-6-[3-(cyclopropylamino)-2-hydroxypropoxy]-7-methoxyquinoline-3-carboxamide
4-{[3-(aminomethyl)-2-ethyl phenyl]amino}-6-[3-(cyclopropylamino)-2-hydroxypropoxy]-7-methoxyquinoline-3-carboxamide
4-({3-[(acetylamino)methyl]-2-ethylphenyl}amino)-6-{3-[acetyl(cyclopropyl)amino]-2-hydroxypropoxy}-7-methoxyquinoline-3-carboxamide
6-[3-(cyclopropylamino)-2-hydroxypropoxy]-4-{[2-ethyl-3-(1H-imidazol-1-ylmethyl)phenyl]amino}-7-methoxyquinoline-3-carboxamide
6-[3-(cyclopropylamino)-2-hydroxypropoxy]-4-{[2-ethyl-3-(1H-pyrazol-1-ylmethyl)phenyl]amino}-7-methoxyquinoline-3-carboxamide
6-{[(2S)-3-(cyclopropylamino)-2-hydroxypropyl]oxy}-4-{[2-ethyl-3-(morpholin-4-ylmethyl)phenyl]amino}-7-methoxyquinoline-3-carboxamide
amino{6,7-diethoxy-4-[(2-ethylphenyl)amino]quinolin-3-yl}methanol
6-[3-(cyclopropylamino)propoxy]-4-{[2-ethyl-3-(1H-imidazol-1-ylmethyl)phenyl]amino}-7-methoxyquinoline-3-carboxamide
4-{[2-ethyl-3-(1H-imidazol-1-ylmethyl)phenyl]amino}-6-methoxy-7-(2-methoxyethoxy)quinoline-3-carboxamide
6-(ethylamino)-4-{[2-ethyl-3-(1H-imidazol-1-ylmethyl)phenyl]amino}-7-methoxyquinoline-3-carboxamide
6-[(2,2-dimethoxyethyl)amino]-4-[(2-ethylphenyl)amino]-7-methoxyquinoline-3-carboxamide
6-[(3,3-diethoxypropyl)amino]-4-[(2-ethylphenyl)amino]-7-methoxyquinoline-3-carboxamide
tert-butyl [2-({3-(aminocarbonyl)-4-[(2-ethyl phenyl)amino]-7-methoxyquinolin-6-yl}amino)ethyl]carbamate
tert-butyl {2-[(3-(aminocarbonyl)-4-{[2-ethyl-3-(hydroxymethyl)phenyl]amino}-7-methoxyquinolin-6-yl)amino]ethyl}carbamate
6-{[3-(cyclopropylamino)propyl]amino}-4-[(2-ethylphenyl)amino]-7-methoxyquinoline-3-carboxamide
4-(2,3-dihydro-1H-inden-1-ylamino)-6,7-dimethoxyquinoline-3-carboxamide
6,7-diethoxy-4-[(2-methylcyclohexyl)amino]quinoline-3-carboxamide
4-{[(3S)-1-(cyanoacetyl)pyrrolidin-3-yl]amino}-6,7-dimethoxyquinoline-3-carboxamide
4-{[(3S)-1-(cyanoacetyl)piperidin-3-yl]amino}-6,7-dimethoxyquinoline-3-carboxamide or a pharmaceutically acceptable salt or solvate thereof.

15. A pharmaceutical composition comprising a compound as claimed in claim 1, or a pharmaceutically acceptable salt thereof, in association with a pharmaceutically acceptable adjuvant, diluent or carrier.

16-22. (canceled)

23. A process for preparing a compound of formula (I) according to claim 1, or a pharmaceutically acceptable salt thereof, which comprises:
(a) reaction of a compound of formula (II): 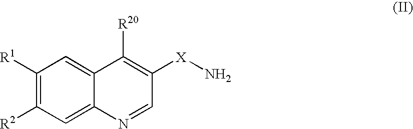
in which R¹ and R² are as defined in claim 1 or are protected derivatives thereof and R²⁰ is a leaving group, with a compound of formula (III):
R^{x}—NH₂ (III)
in which R^{x} is as defined for formula (I) for claim 1 or a protected derivative thereof, or
(b) for compounds of formula (I) where R¹ and/or R² are groups Y(CR³₂)ₚNR⁴R⁵, Y(CR³₂)ₚCONR⁴R⁵, Y(CR³)ₚCO₂R⁶, Y(CR³₂)ₚOR⁶ or Y(CR³₂)ₚR⁶ where Y is oxygen and R³, R⁴, R⁵ and R⁶ are as defined in claim 1, reaction of a compound of formula (IV): 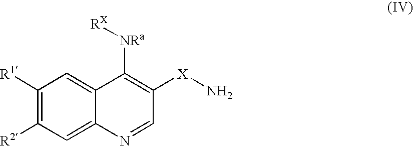
where the R^{1′} or R^{2′} to be converted into a group Y(CR³₂)ₚNR⁴R⁵, Y(CR³₂)ₚCONR⁴R⁵, Y(CR³)ₚCO₂R⁶, Y(CR³₂)ₚOR⁶ or Y(CR³₂)ₚR⁶ is hydroxy and the other R^{1′} or R^{2′} together with R^{x} are as defined above for process (a) with a compound of formula (V):
L-(CR³₂)ₚR²¹ (V)
where R²¹ is NR⁴R⁵, CONR⁴R⁵, CO₂R⁶, OR⁶ or R⁶ and R⁴, R⁵ and R⁶ are as defined in formula (I) in claim 1 or are protected derivatives thereof,
and optionally thereafter process (a) or (b)
removing any protecting groups
converting a compound of formula (I) into a further compound of formula (I)
forming a pharmaceutically acceptable salt or solvate.

24. A method of inhibiting JAK3 receptor kinase activity in a warm-blooded animal in need of such treatment, which method comprises administering to said animal an effective amount of a compound of the formula (I) according to claim 1, or a pharmaceutically acceptable salt or solvate thereof.
